(19) United States
(12) Patent Application Publication
(10) Pub. No.: US 2026/0234295 A1
(43) Pub. Date: Aug. 13, 2026

(54) ULTRALOW MOLECULAR WEIGHT HEPARIN

(71) Applicants: The University of North Carolina at Chapel Hill, Chapel Hill, NC (US); Beth Israel Deaconess Medical Center, Inc., Boston, MA (US)

(72) Inventors: Elliot L. Chaikof, Newton, MA (US); Jian Liu, Chapel Hill, NC (US); Eduardo Stancanelli, Chapel Hill, NC (US); Yongmei Xu, Durham, NC (US)

(21) Appl. No.: 19/110,254

(22) PCT Filed: Sep. 11, 2023
(86) PCT No.: PCT/US2023/032412

§ 371 Date: Mar. 10, 2025

Related U.S. Application Data
(60) Provisional application No. 63/405,189, filed on Sep. 9, 2022.

(51) Int. Cl. C08B 37/00; A61K 31/727; A61L 31/10; A61P 7/02; C12P 19/04

(52) U.S. Cl. CPC C08B 37/0075; A61K 31/727; A61L 31/10; A61P 7/02; C12P 19/04

(57) ABSTRACT

Provided herein are heparin oligomers (e.g., heparin oligomers of Formula (I)), pharmaceutically acceptable salts, solvates, hydrates, polymorphs, co-crystals, tautomers, stereoisomers, isotopically labeled derivatives, and prodrugs thereof, compounds thereof, polymer conjugates thereof, oligosaccharide conjugates thereof, and methods of synthesis. Also provided herein are pharmaceutical compositions, surface coatings, devices, and kits including a heparin oligomer provided herein, or a pharmaceutically acceptable salt, solvate, hydrate, polymorph, co-crystal, tautomer, stereoisomer, isotopically labeled derivative, or prodrug thereof. Also provided herein are methods of using a heparin oligomer (or a related compound, conjugate, or pharmaceutical composition thereof) as provided herein, or a pharmaceutically acceptable salt, solvate, hydrate, polymorph, co-crystal, tautomer, stereoisomer, isotopically labeled derivative, or prodrug thereof, for treating disease or reducing or inhibiting thrombus formation.

### CROSS REFERENCE TO RELATED APPLICATIONS

This application claims benefit of and priority to U.S. Provisional Patent Application Ser. No. 63/405,189, filed Sep. 9, 2022, herein incorporated by reference in its entirety.

### GOVERNMENT SUPPORT

This invention was made with government support under Grant Nos. HL143365, HL094463, and HL144970, awarded by the National Institutes of Health. The government has certain rights in the invention.

### TECHNICAL FIELD

The subject matter disclosed herein relates generally to heparin oligomers, their synthesis, use and to compounds and conjugates comprising the heparin oligomers. More particularly, the subject matter disclosed herein relates to heparin oligomers that are resistant to digestion by heparanase.

### BACKGROUND

Heparin is a naturally occurring glycosaminoglycan, which is also used therapeutically as an anticoagulant. Heparin prevents formation and growth of blood clots and activates lysis mechanisms to break down existing clots. However, degradation of heparin negates its anticoagulant effects.

Heparanase, an endo-β-D-glucuronidase produced by a variety of cells and tissues, cleaves the glycosidic linkage between glucuronic acid (GlcA) and a 3-O- or 6-O-sulfated glucosamine, typified by the disaccharide -[GlcA-GlcNS3S6S]-, which is found within the antithrombin binding domain of heparin. As such, current forms of heparin are susceptible to degradation by heparanase with neutralization of anticoagulant properties.

Accordingly, there is an ongoing need for heparin analogs with anticoagulant properties and resistance to heparanase, particularly for low or ultralow molecular weight, heparanase-resistant heparin analogs.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides a heparin oligomer having a structure of Formula (I):

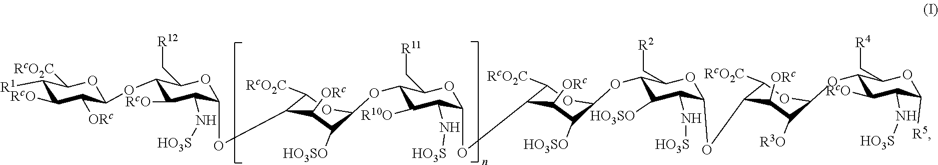

or a pharmaceutically acceptable salt, solvate, hydrate, polymorph, co-crystal, tautomer, stereoisomer, isotopically labeled derivative, or prodrug thereof, wherein: n is 0, 1, 2, 3, 4, 5, 6, 7, 8, 9, or 10; R¹ is —OR^{A}, —SR^{A}, —N(R^{A})₂, halogen, an optionally substituted monosaccharide, or an optionally substituted oligosaccharide; each occurrence of R^{A} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atoms to form an optionally substituted heterocyclic ring or optionally substituted heteroaryl ring; each of R², R⁴, R¹¹, and R¹² is independently —H, —OH, —OSO₃H, or —SO₃H; each of R³ and R¹⁰ is independently —SO₃H, —H, optionally substituted C₁-C₆ alkyl, or an oxygen protecting group; and R⁵ is —OR^{B}, —SR^{B}, —N(R^{B})₂, halogen, an optionally substituted monosaccharide, or an optionally substituted oligosaccharide; each occurrence of R^{B} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{B} are joined together with their intervening atoms to form an optionally substituted heterocyclic ring or optionally substituted heteroaryl ring; and each occurrence of R^{C} is independently —H, optionally substituted C₁-C₆ alkyl, or an oxygen protecting group; provided that one of R¹ and R⁵ is an optionally substituted monosaccharide.

In some embodiments, R¹¹ is —H, —OH, or —OSO₃H. In some embodiments, R¹⁰ is —H or —SO₃H. In some embodiments, n is 0, 1, or 2.

In some embodiments, the heparin oligomer has a structure of Formula (I-A):

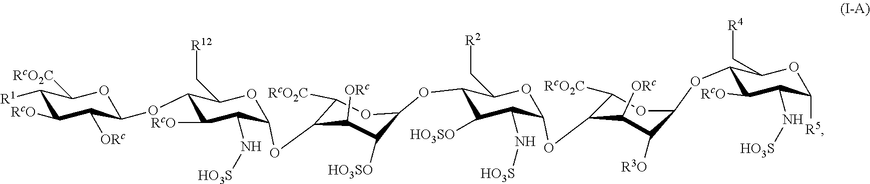

or a pharmaceutically acceptable salt, solvate, hydrate, polymorph, co-crystal, tautomer, stereoisomer, isotopically labeled derivative, or prodrug thereof, wherein: R¹ is —OR^{A}, —SR^{A}, —N(R^{A})₂, halogen, an optionally substituted monosaccharide, or an optionally substituted oligosaccharide; each occurrence of R^{A} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atoms to form an optionally substituted heterocyclic ring or optionally substituted heteroaryl ring; each of R², R⁴, and R¹² is independently —H, —OH, —OSO₃H, or —SO₃H; R³ is —H, —SO₃H, optionally substituted C₁-C₆ alkyl, or an oxygen protecting group; and R⁵ is —OR^{B}, —SR^{B}, —N(R^{B})₂, halogen, or an optionally substituted monosaccharide; each occurrence of R^{B} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{B} are joined together with their intervening atoms to form an optionally substituted heterocyclic ring or optionally substituted heteroaryl ring; and each occurrence of R^{C} is independently —H, optionally substituted C₁-C₆ alkyl, or an oxygen protecting group; provided that one of R¹ and R⁵ is an optionally substituted monosaccharide.

In some embodiments, R² is —H, —OH, or —OSO₃H. In some embodiments, R² is —OSO₃H. In some embodiments, R³ is —H or —SO₃H. In some embodiments, R³ is —SO₃H. In some embodiments, R⁴ is —H, —OH, or —OSO₃H. In some embodiments, R⁴ is —OSO₃H. In some embodiments, R¹² is —H, —OH, or —OSO₃H. In some embodiments, R¹² is —OSO₃H.

In some embodiments, the heparin oligomer has a structure of a formula selected from the group comprising:

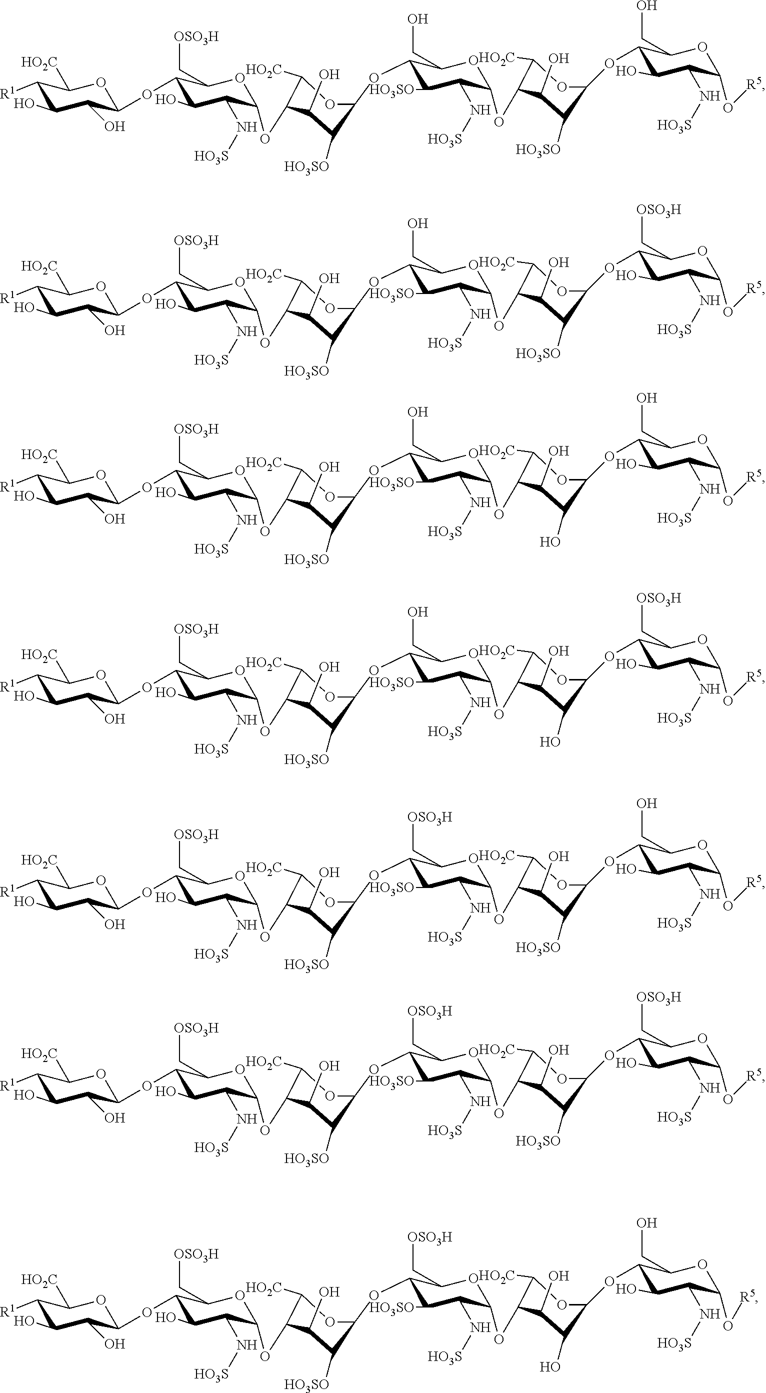

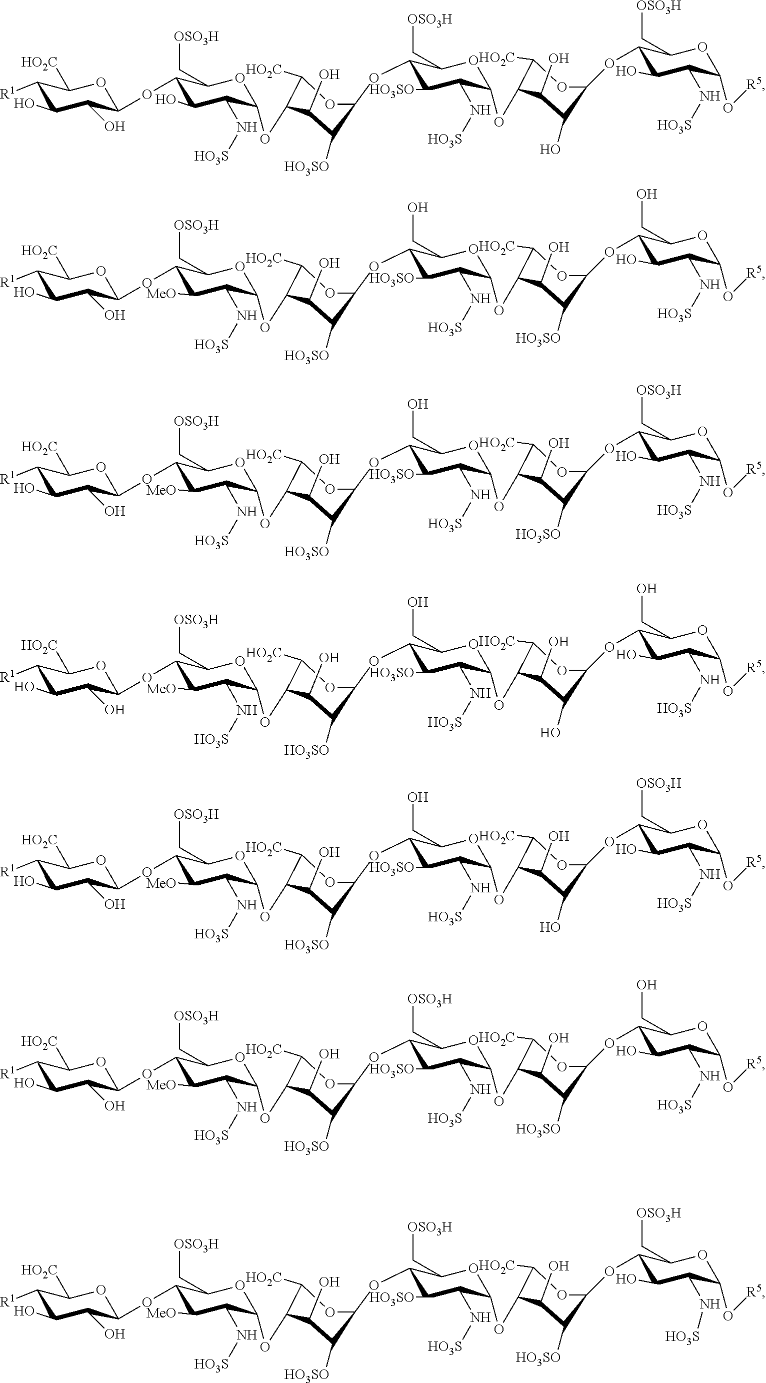

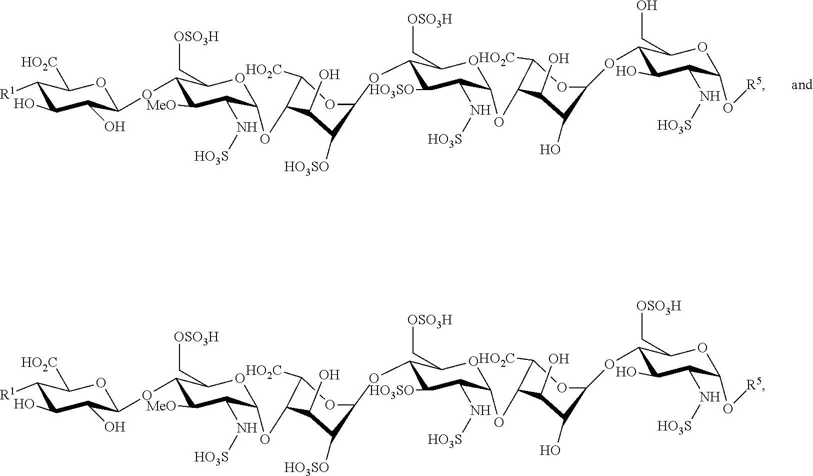

or a pharmaceutically acceptable salt, solvate, hydrate, polymorph, co-crystal, tautomer, stereoisomer, isotopically labeled derivative, or prodrug thereof. In some embodiments, the heparin oligomer has a structure of the formula:

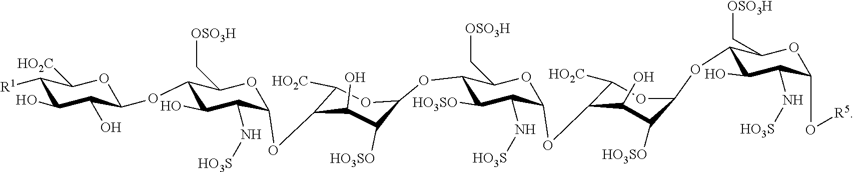

In some embodiments, R¹ is —OR^{A}, —SR^{A}, —N(R^{A})₂, or halogen. In some embodiments, R^{A} is —H, a nitrogen protecting group when attached to a nitrogen atom, an oxygen protecting group when attached to an oxygen atom, or a sulfur protecting group when attached to a sulfur atom.

In some embodiments, R¹ is an optionally substituted monosaccharide. In some embodiments, R¹ is optionally substituted glucosamine. IN some embodiments, R¹ is

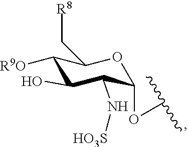

wherein: R⁸ is —H, —OH, —OSO₃H, or —SO₃H; and R⁹ is —H, an oxygen protecting group, optionally substituted aliphatic, optionally substituted heteroaliphatic, optionally substituted carbocyclyl, optionally substituted heterocyclyl, optionally substituted aryl, or optionally substituted heteroaryl. In some embodiments, R⁸ is —H, —OH, or —OSO₃H. In some embodiments, R⁹ is —H or an oxygen protecting group.

In some embodiments, R¹ is an optionally substituted oligosaccharide. In some embodiments, the optionally substituted oligosaccharide comprises a pentasaccharide moiety with the structure:

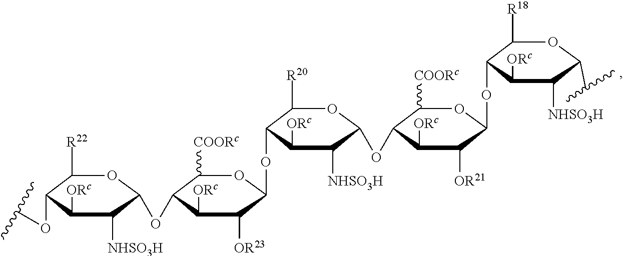

wherein: each of R¹⁸, R²⁰, and R²² is independently —H, —OH, —OSO₃H, or —SO₃H; each of R²¹ and R²³ is independently —SO₃H, —H, optionally substituted C₁-C₆ alkyl, or an oxygen protecting group; and each occurrence of R^{C} is independently —H, optionally substituted C₁-C₆ alkyl, or an oxygen protecting group.

In some embodiments, R¹ is an optionally substituted monosaccharide or an optionally substituted oligosaccharide having a structure of the formula:

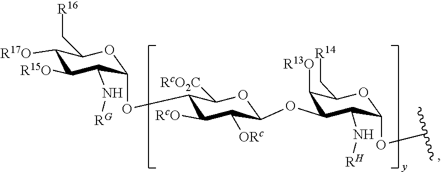

wherein: y is 0, 1, 2, 3, 4, 5, or 6; each of R¹³, R¹⁵, and R¹⁷ is independently —SO₃H, —H, optionally substituted C₁-C₆ alkyl, or an oxygen protecting group; and each R¹⁴ is independently —OH, an oxygen protecting group, optionally substituted C₁-C₆ alkyl, or —OSO₃H; R¹⁶ is —OH, an oxygen protecting group, optionally substituted C₁-C₆ alkyl, or —OSO₃H; each occurrence of R^{C} is independently —H, optionally substituted C₁-C₆ alkyl, or an oxygen protecting group; R^{H} is optionally substituted acyl; and R^{G} is optionally substituted acyl or optionally substituted alkyl. In some embodiments, y is 2. In some embodiments, R¹³, R¹⁵, and R¹⁷ are each H. In some embodiments, each R¹⁴ is OH. In some embodiments, each R¹⁶ is OH. In some embodiments, each R^{H} is —C(═O)CH₃. In some embodiments, R^{G} is C(═O)-(substituted alkyl), wherein substituted alkyl is alkyl substituted with —N₃ or —C≡C—H. In some embodiments, R^{G} is optionally substituted alkyl or optionally substituted acyl, wherein the optionally substituted alkyl or optionally substituted acyl is alkyl or acyl substituted with a linker, wherein said linker is covalently bonded to an optionally substituted oligosaccharide. In some embodiments, the linker is —NHC(═O)—, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or any combination thereof. In some embodiments, the linker is covalently bonded to an optionally substituted oligosaccharide having a structure of Formula (II):

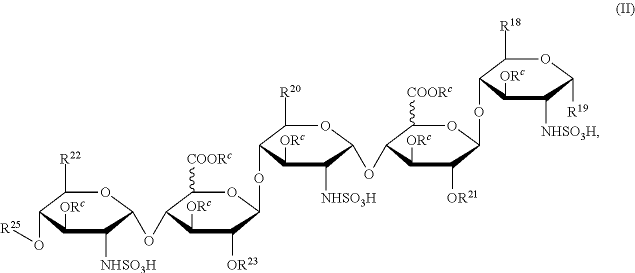

wherein: each of R¹⁸, R²⁰, and R²² is independently —H, —OH, —OSO₃H, or —SO₃H; each of R²¹ and R²³ is independently —SO₃H, —H, optionally substituted C₁-C₆ alkyl, or an oxygen protecting group; each occurrence of R^{C} is independently —H, optionally substituted C₁-C₆ alkyl, or an oxygen protecting group; R¹⁹ is a covalent bond to the linker or a bivalent group covalently bonded to the linker, wherein the bivalent group covalently bonded to the linker is selected from —O—R²⁶—, —SR²⁶—, —N(R^{B})(R²⁶)—, an optionally substituted monosaccharide residue covalently bonded to the linker, and an optionally substituted oligosaccharide residue covalently bonded to the linker; R²⁶ is a covalent bond to the linker, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene; R^{B} is —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group when attached to a nitrogen atom; or wherein R^{B} and R²⁶ together with the nitrogen atom to which they are attached form an optionally substituted heterocyclic ring or optionally substituted heteroaryl ring that comprises a covalent bond to the linker; and R²⁵ is —H, optionally substituted alkyl, optionally substituted aralkyl, optionally substituted aryl, an optionally substituted monosaccharide, or an optionally substituted oligosaccharide. In some embodiments, R²⁵ comprises one or more optionally substituted galactosamine residues.

In some embodiments, R⁵ is —OH or —O(oxygen protecting group). In some embodiments, R⁵ is an optionally substituted monosaccharide. In some embodiments, R⁵ is an optionally substituted glucuronide. In some embodiments, R⁵ is

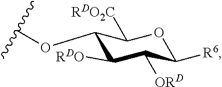

wherein: RD is —H, optionally substituted C₁-C₆ alkyl, or an oxygen protecting group; R⁶ is —OR^{E}, —SR^{E}, or —N(R^{E})₂; and each occurrence of R^{E} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{E} are joined together with their intervening atoms to form an optionally substituted heterocyclic ring or optionally substituted heteroaryl ring. In some embodiments, R⁶ is —OR^{E} or —SR^{E}. In some embodiments, R⁶ is —O-(optionally substituted phenyl). In some embodiments, R⁶ is

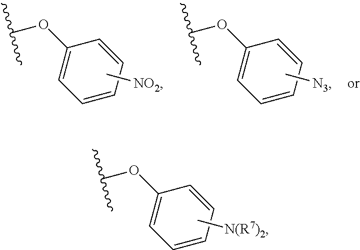

wherein: each occurrence of R⁷ is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or two occurrences of R⁷ are joined together with their intervening atoms to form an optionally substituted heterocyclic ring or optionally substituted heteroaryl ring. In some embodiments, R⁶ is

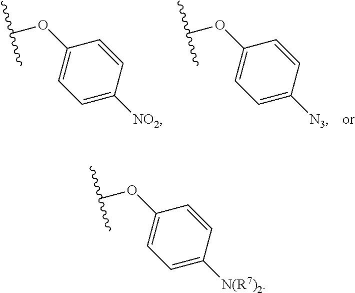

In some embodiments, the heparin oligomer is a heparin heptamer having the structure:

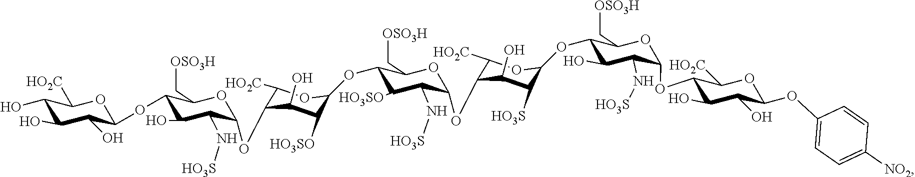

or a pharmaceutically acceptable salt, solvate, hydrate, polymorph, co-crystal, tautomer, stereoisomer, isotopically labeled derivative, or prodrug thereof. In some embodiments, the heparin oligomer has the structure of compound 3 or compound 6, or a pharmaceutically acceptable salt, solvate, hydrate, polymorph, co-crystal, tautomer, stereoisomer, isotopically labeled derivative, or prodrug thereof, wherein the structure of compound 3 is

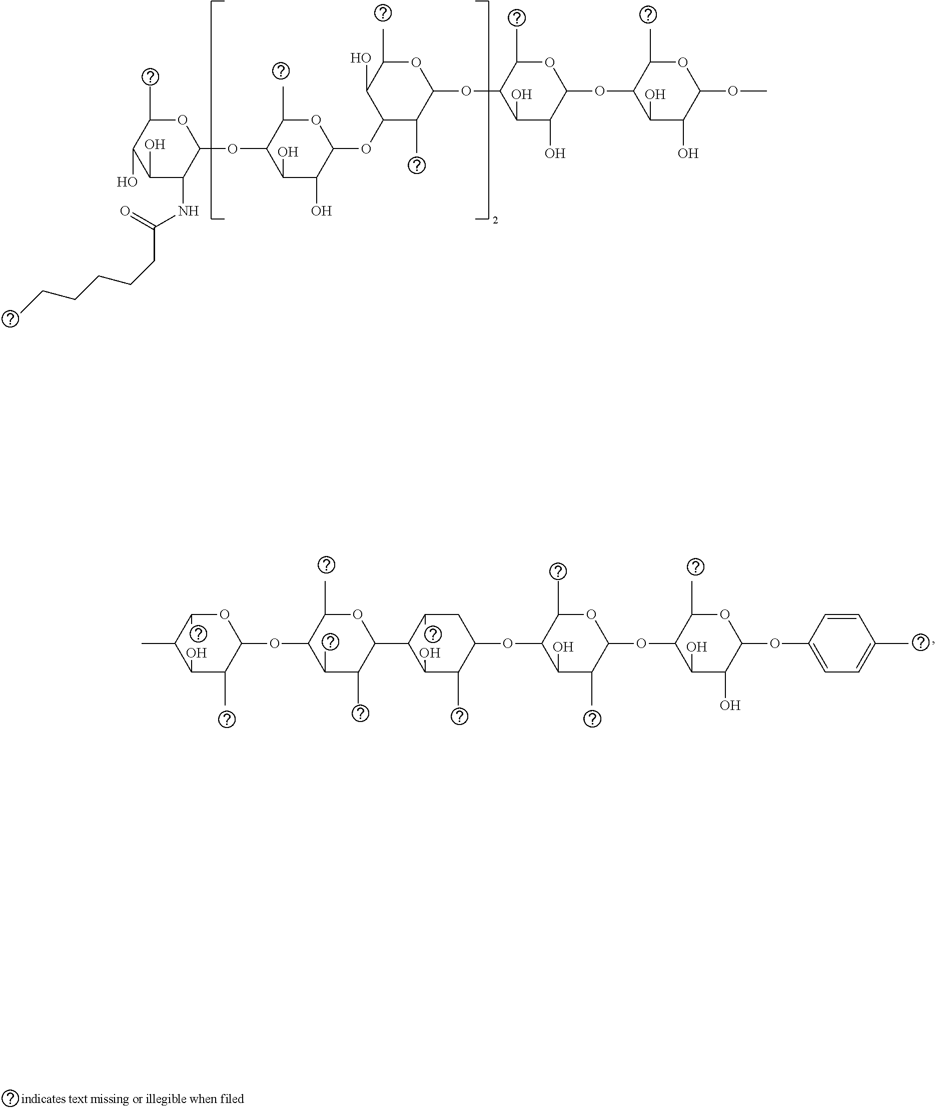

and the structure of compound 6 is:

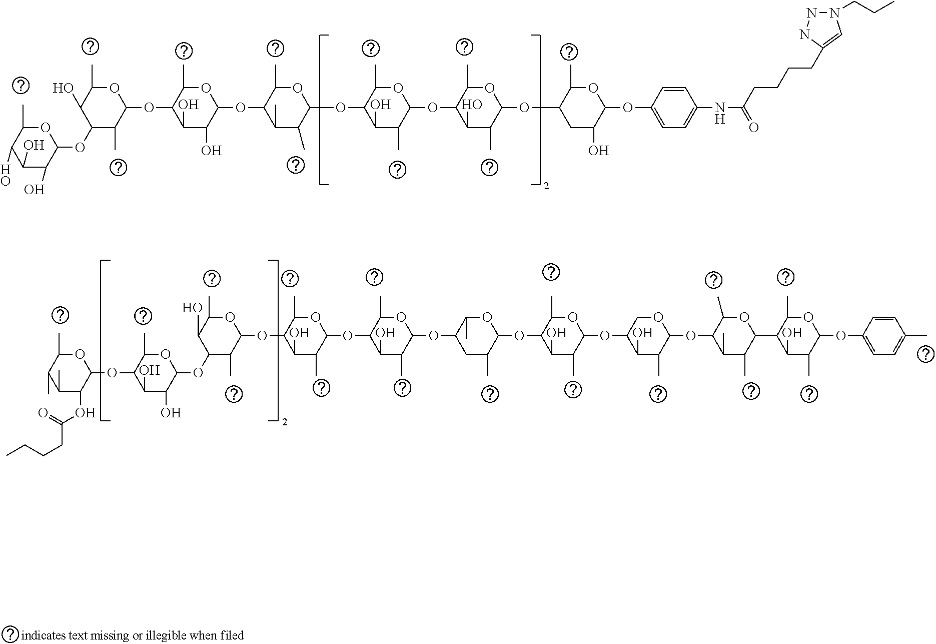

In some embodiments, the heparin oligomer is resistant to heparanase degradation. In some embodiments, the heparin oligomer has anti-FXa activity and/or anti-FIIa activity.

In some embodiments, the presently disclosed subject matter provides an oligomeric compound comprising two or more repeat units connected via a linker, or a pharmaceutically acceptable salt, solvate, hydrate, polymorph, co-crystal, tautomer, stereoisomer, isotopically labeled derivative, or prodrug thereof, wherein the repeat units are each independently a heparin oligomer of Formula (I). In some embodiments, the linker is a bond, an optionally substituted saccharide, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or any combination thereof. In some embodiments, the oligomeric compound has a linear structure. In some embodiments, the oligomeric compound comprises five or more heparin oligomers.

In some embodiments, the presently disclosed subject matter provides a polymer conjugate, or a pharmaceutically acceptable salt, solvate, hydrate, polymorph, co-crystal, tautomer, stereoisomer, isotopically labeled derivative, or prodrug thereof, comprising a heparin oligomer of Formula (I), or a pharmaceutically acceptable salt, solvate, hydrate, polymorph, co-crystal, tautomer, stereoisomer, isotopically labeled derivative, or prodrug thereof, conjugated to a polymer via a linker. In some embodiments, the linker is a bond, an optionally substituted monosaccharide, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or any combination thereof. In some embodiments, the linker is a bond, —NHC(═O)—, or an optionally substituted saccharide. In some embodiments, the polymer is conjugated to either terminus of the heparin oligomer. In some embodiments, R⁶ of the heparin oligomer is —O(optionally substituted phenyl) substituted with the linker. In some embodiments, the polymer conjugate has the structure:

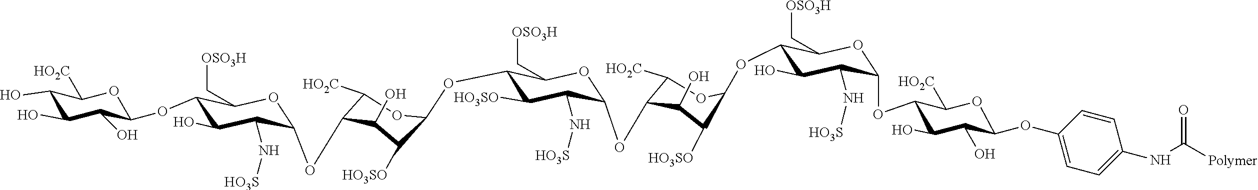

or a pharmaceutically acceptable salt, solvate, hydrate, polymorph, co-crystal, tautomer, stereoisomer, isotopically labeled derivative, or prodrug thereof. In some embodiments, the polymer conjugate has the structure:

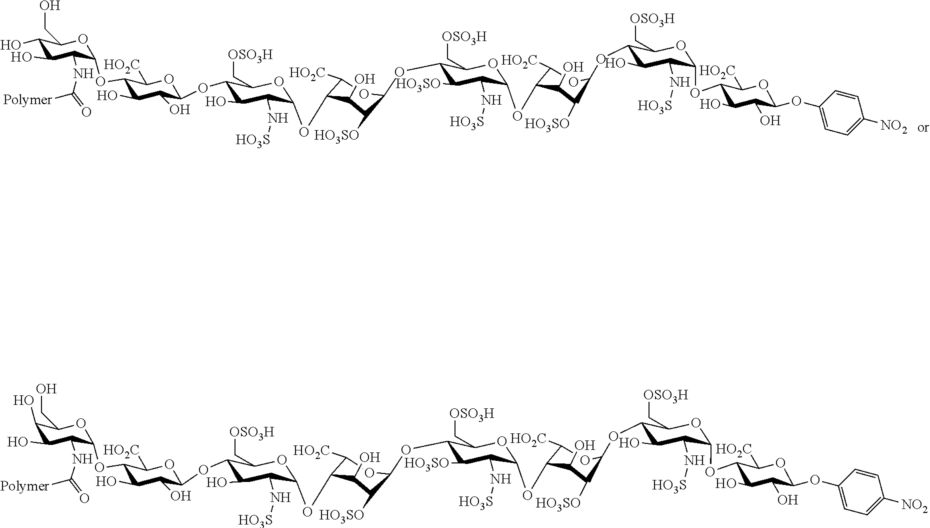

or a pharmaceutically acceptable salt, solvate, hydrate, polymorph, co-crystal, tautomer, stereoisomer, isotopically labeled derivative, or prodrug thereof. In some embodiments, the polymer is a polyethylene glycol, a polyacrylate, a polyester, a polycarbonate, a polyolefin, a polyamide, or any combination thereof. In some embodiments, the polymer comprises one or more additional instances of a heparin oligomer of Formula (I). In some embodiments, the polymer comprises one or more additional instances of the heparin oligomer grafted onto a polymer backbone.

In some embodiments, the presently disclosed subject matter provides an oligosaccharide conjugate comprising the structure:

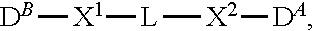

wherein: L is a bivalent linker; X¹ is present or absent and when present is an optionally substituted monosaccharide residue or an optionally substituted oligosaccharide residue; X² is present or absent and when present is an optionally substituted monosaccharide residue or an optionally substituted oligosaccharide residue; D^{A} is a heparin oligomer having a structure of Formula (I-B):

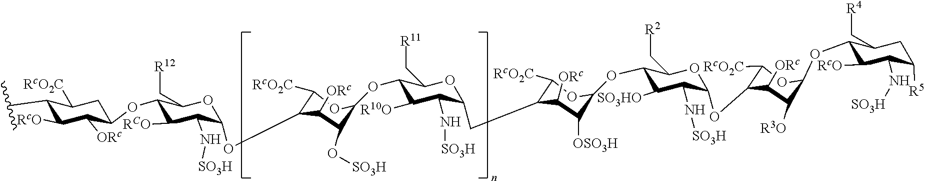

(I-B); D^{B} is an oligosaccharide-containing oligomer having a structure of Formula (II-A):

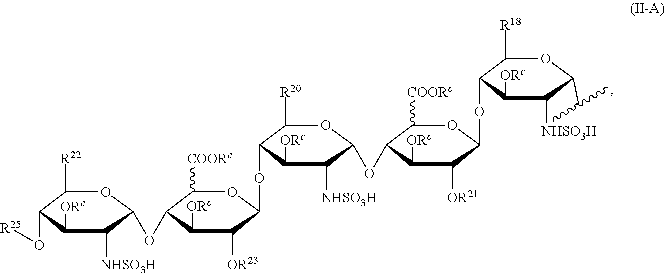

n is 1, 2, 3, 4, 5, 6, 7, 8, 8, 9, or 10; each of R², R⁴, R¹¹, R¹², R¹⁸, R²⁰, and R²² is independently —H, —OH, —OSO₃H, or —SO₃H; each of R³, R¹⁰, R²¹, and R²³ is independently —SO₃H, —H, optionally substituted C₁-C₆ alkyl, or an oxygen protecting group; and R⁵ is —OR^{B}, —SR^{B}, —N(R^{B})₂, halogen, an optionally substituted monosaccharide, or an optionally substituted oligosaccharide; each occurrence of R^{B} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{B} are joined together with their intervening atoms to form an optionally substituted heterocyclic ring or optionally substituted heteroaryl ring; each occurrence of R^{C} is independently —H, optionally substituted C₁-C₆ alkyl, or an oxygen protecting group; R²⁵ is —H, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aralkyl, optionally substituted aryl, optionally substituted heteroaryl, an oxygen protecting group, an optionally substituted monosaccharide, or an optionally substituted oligosaccharide. In some embodiments, L is an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or any combination thereof. In some embodiments, L is: —O-phenyl-NH—C(═O)-(alkylene)-(triazolyl)-(alkylene)-C(═O)—. In some embodiments, X² is present and comprises one or more optionally substituted galactosamine residues. In some embodiments, R¹⁵ is an optionally substituted oligosaccharide comprising one or more optionally substituted galactosamine residues. In some embodiments, D^{A} comprises the structure:

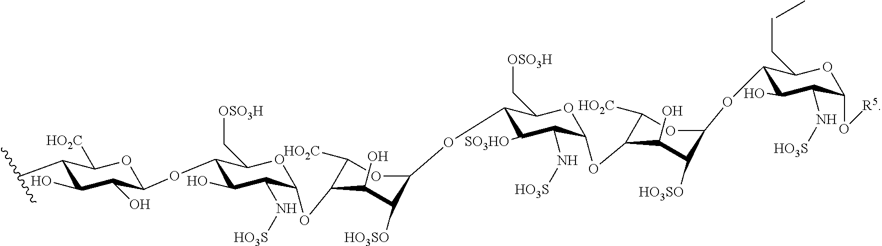

In some embodiments, D^{B} comprises the structure:

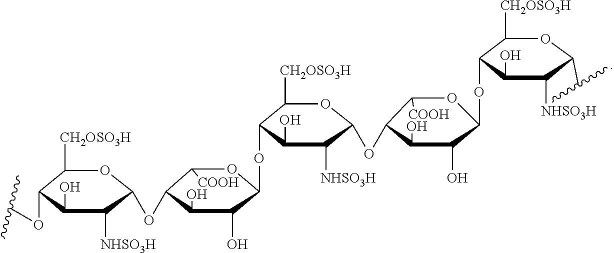

In some embodiments, the oligosaccharide conjugate has the structure:

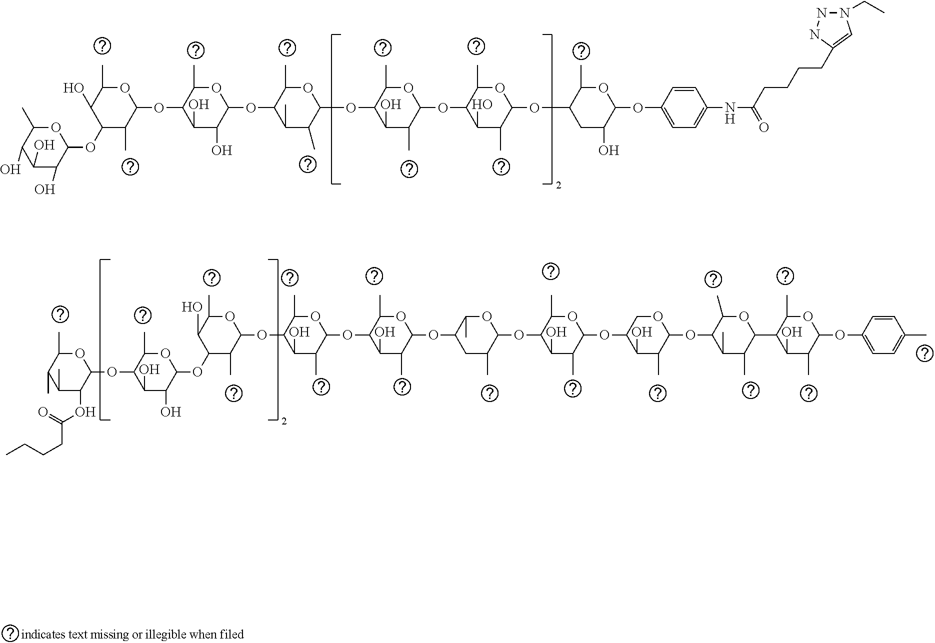

In some embodiments, the presently disclosed subject matter provides a method of synthesizing a heparin oligomer of Formula (I), or a pharmaceutically acceptable salt, solvate, hydrate, polymorph, co-crystal, tautomer, stereoisomer, isotopically labeled derivative, or prodrug thereof, comprising the sequential steps of: (a) elongating a saccharide using: (i) recombinant Pasteurella multocida heparosan synthase (pmHS2) and uridine 5-diphospho-N-trifluoroacetyl glucosamine (UDP-GlcNTFA); and (ii) recombinant Pasteurella multocida heparosan synthase (pmHS2) and uridine 5-diphospho-N-glucuronic acid (UDP-GlcA) in either order, one or more times, to obtain a trifluoroacetate-protected heparin hexamer oligosaccharide intermediate comprising the structure

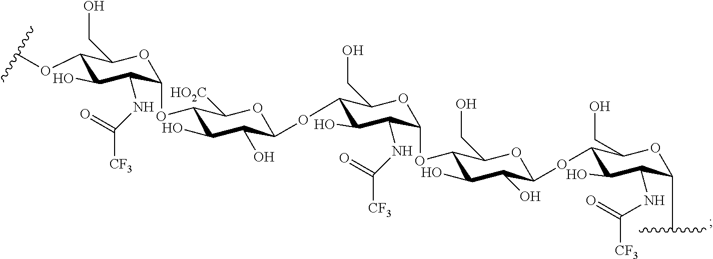

(b) detrifluoroacetylating the heparin hexamer oligosaccharide intermediate of step (a) to obtain a heparin hexamer oligosaccharide intermediate comprising the structure

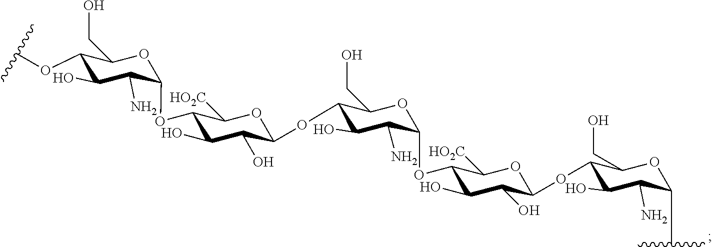

(c) N-sulfating the heparin hexamer oligosaccharide intermediate of step (b) to obtain an NS-hexamer oligosaccharide intermediate comprising the structure

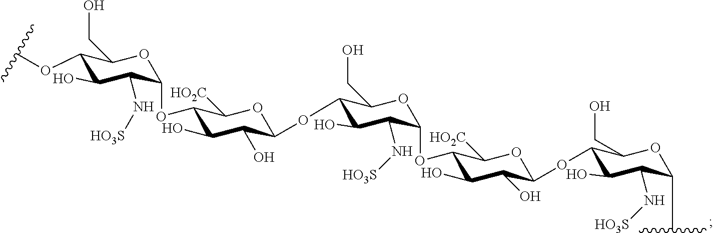

(d) elongating the NS-hexamer oligosaccharide intermediate of step (c) with recombinant Pasteurella multocida heparosan synthase (pmHS2) and uridine 5-diphospho-N-glucuronic acid (UDP-GlcA) to obtain an oligosaccharide intermediate comprising the structure

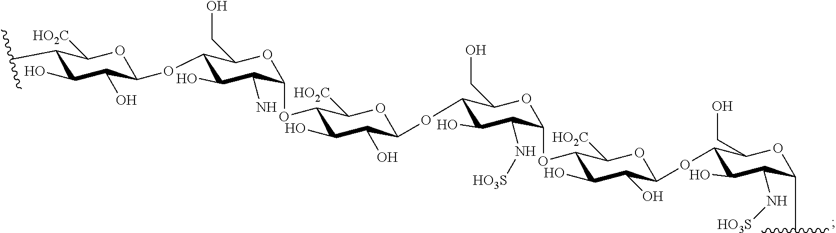

(e) converting glucuronic acid to 2-O-sulfated iduronic acid to obtain an oligosaccharide intermediate comprising the structure

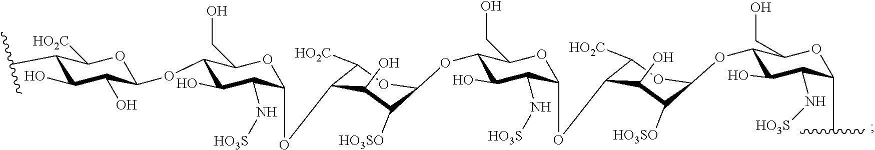

and optionally (f) performing 3-O- and/or 6-O sulfation of the oligosaccharide intermediate of step (e).

In some embodiments, the saccharide of step (a) is para-nitrophenyl glucuronide. In some embodiments, step (b) comprises reaction under basic conditions. In some embodiments, step (c) comprises incubation with 3-morpholino-propane-1-sulfonic acid, N-sulfotransferase, and 3′-phosphoadenosine 5′-phosphosulfate. In some embodiments, step (e) comprises incubation with C₅-epimerase, 2-O-sulfotransferase, and 3′-phosphoadenosine 5′-phosphosulfate in 3-morpholino-propane-1-sulfonic acid buffer. In some embodiments, step (f) comprises incubation with 3-O-sulfotransferase 3 and 3′-phosphoadenosine 5′-phosphosulfate and/or incubation with 6-sulfotransferase 3 in 3-morpholino-propane-1-sulfonic acid buffer. In some embodiments, any of steps (a)-(f) is followed by an additional purification step. In some embodiments, the heparin oligomer is synthesized in a final yield of about 45% over all steps.

In some embodiments, the presently disclosed subject matter provides a pharmaceutical composition comprising a heparin oligomer of Formula (I), an oligomeric compound thereof, a polymer conjugate thereof, or an oligosaccharide conjugate thereof, or a pharmaceutically acceptable salt, solvate, hydrate, polymorph, co-crystal, tautomer, stereoisomer, isotopically labeled derivative, or prodrug thereof, and a pharmaceutically acceptable excipient. In some embodiments, the pharmaceutical composition is formulated for oral administration. In some embodiments, the pharmaceutical composition is formulated for intravenous or subcutaneous administration. In some embodiments, the pharmaceutical composition further comprises an additional therapeutic agent.

In some embodiments, the presently disclosed subject matter provides a surface coating comprising a heparin oligomer of Formula (I), an oligomeric compound thereof, a polymer conjugate thereof, or an oligosaccharide conjugate thereof, or a pharmaceutically acceptable salt, solvate, hydrate, polymorph, co-crystal, tautomer, stereoisomer, isotopically labeled derivative, or prodrug thereof, and an excipient.

In some embodiments, the presently disclosed subject matter provides a device comprising a surface coating comprising a heparin oligomer of Formula (I), an oligomeric compound thereof, a polymer conjugate thereof, or an oligosaccharide conjugate thereof, or a pharmaceutically acceptable salt, solvate, hydrate, polymorph, co-crystal, tautomer, stereoisomer, isotopically labeled derivative, or prodrug thereof, and an excipient. In some embodiments, the device is an implantable medical device. In some embodiments, the device is a vascular graft, a stent, a cardiopulmonary bypass circuit, a ventricular assist device, or a respiratory support system.

In some embodiments, the presently disclosed subject matter provides a method of treating or preventing a disease in a subject in need thereof, comprising administering to the subject an effective amount of a heparin oligomer of Formula (I), an oligomeric compound thereof, a polymer conjugate thereof, or an oligosaccharide conjugate thereof, or a pharmaceutically acceptable salt, solvate, hydrate, polymorph, co-crystal, tautomer, stereoisomer, isotopically labeled derivative, or prodrug thereof, or a pharmaceutical composition thereof. In some embodiments, the disease is cardiovascular disease, atherosclerosis, atherosclerotic lesions, thrombus formation, thromboembolism, stroke, or myocardial infarction. In some embodiments, the disease is primary or recurrent venous thromboembolism (VTE). In some embodiments, the venous thromboembolism is deep vein thrombosis, pulmonary embolism, or non-occlusive venous thrombosis.

In some embodiments, the method reduces thrombus formation. In some embodiments, the method reduces thrombus formation by at least about 10%, at least about 15%, at least about 20%, at least about 25%, at least about 30%, at least about 35%, or at least about 40%.

In some embodiments, the subject exhibits a prothrombotic phenotype or has elevated heparanase expression, plasma heparanase levels, plasma heparan sulfate concentrations, D-dimer levels, or procoagulant activity. In some embodiments, the subject has or has been diagnosed with renal insufficiency, type 2 diabetes, a gastrointestinal malignancy, an inflammatory disease, cancer, or a metastatic disease. In some embodiments, the inflammatory disease is inflammatory bowel disease, rheumatoid arthritis, or atherosclerosis. In some embodiments, the cancer is lung cancer, breast cancer, colorectal cancer, or pancreatic cancer. In some embodiments, the subject is after surgery, takes oral contraceptives, or has a history of prosthetic valve thrombosis.

In some embodiments, the method further comprises administering an additional therapy or therapeutic agent to the subject before administering the effective amount of the heparin oligomer; oligomeric compound; polymer conjugate; oligosaccharide conjugate; pharmaceutically acceptable salt, solvate, hydrate, polymorph, co-crystal, tautomer, stereoisomer, isotopically labeled heparin oligosaccharide, or prodrug thereof; or pharmaceutical composition thereof. In some embodiments, the method further comprises administering an additional therapy or therapeutic agent to the subject concurrently with administering the effective amount of the heparin oligomer; oligomeric compound; polymer conjugate; oligosaccharide conjugate; pharmaceutically acceptable salt, solvate, hydrate, polymorph, co-crystal, tautomer, stereoisomer, isotopically labeled heparin oligosaccharide, or prodrug thereof, or pharmaceutical composition thereof. In some embodiments, the method further comprises administering an additional therapy or therapeutic agent to the subject after administering the effective amount of the heparin oligomer; oligomeric compound; polymer conjugate; oligosaccharide conjugate; pharmaceutically acceptable salt, solvate, hydrate, polymorph, co-crystal, tautomer, stereoisomer, isotopically labeled heparin oligosaccharide, or prodrug thereof, or pharmaceutical composition thereof.

In some embodiments, the presently disclosed subject matter provides for the use of a heparin oligomer of Formula (I), an oligomeric compound thereof, a polymer conjugate thereof, or an oligosaccharide conjugate thereof, or a pharmaceutically acceptable salt, solvate, hydrate, polymorph, co-crystal, tautomer, stereoisomer, isotopically labeled heparin oligosaccharide, or prodrug thereof, or a pharmaceutical composition thereof, for the manufacture of a medicament for treating or preventing a disease in a subject in need thereof.

In some embodiments, the presently disclosed subject matter provides a heparin oligomer of Formula (I), an oligomeric compound thereof, a polymer conjugate thereof, or an oligosaccharide conjugate thereof, or a pharmaceutically acceptable salt, solvate, hydrate, polymorph, co-crystal, tautomer, stereoisomer, isotopically labeled heparin oligosaccharide, or prodrug thereof, or a pharmaceutical composition thereof, for use in treating or preventing a disease in a subject in need thereof.

In some embodiments, the presently disclosed subject matter provides a kit comprising: a heparin oligomer of Formula (I), an oligomeric compound thereof, a polymer conjugate thereof, or an oligosaccharide conjugate thereof, or a pharmaceutically acceptable salt, solvate, hydrate, polymorph, co-crystal, tautomer, stereoisomer, isotopically labeled derivative, or prodrug thereof, or a pharmaceutical composition thereof; and instructions for administering to a subject the heparin oligomer, oligomeric compound, polymer conjugate, oligosaccharide conjugate, pharmaceutically acceptable salt, solvate, hydrate, polymorph, co-crystal, tautomer, stereoisomer, isotopically labeled derivative, prodrug, or the pharmaceutical composition.

Accordingly, it is an object of the presently disclosed subject matter to provide heparin oligomers of Formula (I), as well as related compounds, conjugates, pharmaceutical compositions and methods.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A-1D show the route for the chemoenzymatic synthesis of heparanase-resistant (HR) 7-mer and heparanase-sensitive (HS) 6-mer. FIG. 1A shows the synthesis of a nitrogen-sulfated (NS)-6-mer intermediate. FIG. 1B shows a HR 7-mer was synthesized from a common intermediate. FIG. 1C shows a HS 6-mer was synthesized from a common NS-6-mer intermediate. FIG. 1D shows the chemical structures of HR 7-mer and HS 6-mer. The trisaccharide domain in HR 7-mer and in HS 6-mer is highlighted with a horizontal bar. Abbreviations: pmHS2, Pasteurella multocida heparosan synthase-2; UDP-GlcNTFA, uridine diphospho N-trifluoroacetyl glucosamine; UDP-GlcA, uridine diphospho glucuronic acid; NST, N-sulfotransferase; C5-epi, C₅-epimerase; 2-OST, 2-O-sulfotransferase; 6-OST, 6-O-sulfotransferase 3; PAPS, 3′-phosphoadenosine 5′-phosphosulfate; 3-OST-3, 3-O-sulfotransferase-3; and 3-OST-1, 3-O-sulfotransferase-1.

FIGS. 2A-2C show high-performance liquid chromatography (HPLC) analysis of heparanase-sensitive (HS) 6-mer and heparanase-resistant (HR) 7-mer with or without heparanase digestion. FIG. 2A shows digestion of HS 6-mer by heparanase. FIG. 2B shows HPLC chromatograms of HS 6-mer and HR 7-mer before (top) and after (bottom) overnight incubation with heparanase. FIG. 2C shows liquid chromatography-mass spectrometry (LC-MS) analysis of HS 6-mer and HR 7-mer before (top) and after (bottom) digestion by heparanase.

FIGS. 3A-3B show loss of anti-factor Xa (anti-FXa) activity of ultralow molecular weight heparins in response to heparanase exposure. FIG. 3A shows anti-FXa activity of heparanase-sensitive (HS) 6-mer and the heparanase digested byproduct, 4-mer-D, demonstrating a complete loss of activity. FIG. 3B shows FXa activity of heparanase-resistant (HR) 7-mer and the heparanase digested byproduct, 6-mer-D, with preservation of anti-FXa activity.

FIGS. 4A-4D show evaluation of anticoagulant and antithrombogenic activities of heparanase-resistant (HR) 7-mers in a murine deep vein thrombosis (DVT) model. FIG. 4A shows plasma anti-factor Xa (anti-FXa) activity determined over a 3 hour period after subcutaneous administration of HR 7-mer at 12 micrograms per gram (μg/g) mouse (n≥3 mice/time point). FIG. 4B shows photographs of venous thrombus and FIG. 4C shows thrombus weight (in grams (g)) obtained 48 hours (hr) after electrolytic injury of the vena cava following subcutaneous administration of saline vehicle (n=6), enoxaparin (4 μg/g, 4 hr-pre, n=5), or the ultralow molecular weight heparin, HR 7-mer (12 μg/g, 1 hr-pre, n=5). FIG. 4D shows tail vein bleeding time (in seconds (sec)) measured after the subcutaneous administration of saline (n=5), enoxaparin (4 μg/g, 4 hr-pre, n=7), or HR 7-mer (12 μg/g, 1 hr-pre, n=5).

FIG. 5A shows a reaction schemes for the heparanase digestion of heparanase-sensitive (HS) 6-mer. FIG. 5B shows a reaction scheme for the heparanase digestion of heparanase-resistant (HR) 7-mer.

FIG. 6 shows a comparison of factor Xa (FXa) activities of heparin oligosaccharides. FXa activity (percent inhibition (%)) of enoxaparin, fondaparinux, and heparanase-resistant (HR) 7-mer was measured using the heparin activity assay kit sold under the tradename ACTICHROME® (BioMedica Diagnostics, Windsor, Canada) with calculated 50% inhibitory concentration (IC50) values of 2.46 micrograms per milliliter (μg/mL), 0.37 μg/mL, and 7.92 μg/mL, respectively. Data shown as mean standard deviation. Briefly, varying concentrations of HR 7-mer, enoxaparin, and fondaparinux were prepared in phosphate-buffered saline (PBS). A total of 5 microliters (μL) of each sample was added to each well, followed by the addition of 40 μL of human antithrombin for 2 minutes (min) at 37 degrees Celsius (° C.). A total of 40 μL of bovine FXa stock solution was then added and incubated for 1 min at 37° C., followed by the addition of 40 μL of chromogenic substrate sold under the tradename SPECTROZYME® FXa (BioMedica Diagnostics, Windsor, Canada). After a 5 min incubation period at 37° C., the reaction was terminated by the addition of 40 μL of glacial acetic acid. Absorbance was measured at 405 nanometers (nm) and results presented as percent inhibition of FXa activity.

FIG. 7 shows a route for the synthesis of a heparanase-resistant (HR), azide-functionalized heparin oligomer, referred to herein as Compound 3, which is a synthetic intermediate in the synthesis of an exemplary HR oligosaccharide conjugate, i.e. Compound 6. Abbreviations: pmHS2, Pasteurella multocida heparosan synthase-2; UDP-GlcNTFA, uridine diphospho N-trifluoroacetyl glucosamine; UDP-GlcA, uridine diphospho glucuronic acid; UDP-GalNAc, uridine diphospho N-acetylgalactosamine; 2-OST, 2-O-sulfotransferase; 6-OST, 6-O-sulfotransferase 3; PAPS, 3′-phosphoadenosine 5′-phosphosulfate; 3-OST, 3-O-sulfotransferase; kfoC, E. coli K4 chondroitin synthase; M, molar; and LiOH, lithium hydroxide.

FIG. 8 shows a route for the synthesis of an alkyne-functionalized oligosaccharide-containing compound, referred to herein as Compound 4, a synthetic intermediate in the synthesis of an exemplary heparanase-resistant (HR) oligosaccharide conjugate, i.e., Compound 6. Abbreviations: pmHS2, Pasteurella multocida heparosan synthase-2; UDP-GlcNTFA, uridine diphospho N-trifluoroacetyl glucosamine; UDP-GlcA, uridine diphospho glucuronic acid; UDP-GalNAc, uridine diphospho N-acetylgalactosamine; NST, N-sulfotransferase; 2-OST, 2-O-sulfotransferase; 6-OST, 6-O-sulfotransferase 3; PAPS, 3′-phosphoadenosine 5′-phosphosulfate; 3-OST, 3-O-sulfotransferase; kfoC, E. coli K4 chondroitin synthase; H₂, hydrogen gas; Pd, palladium; M, molar; and LiOH, lithium hydroxide.

FIG. 9 shows a route for the synthesis of heparanase-resistant (HR) oligosaccharide conjugate, referred to herein as Compound 6, from Compound 3 and Compound 4 via an exemplary Click chemistry reaction (more particularly, a copper-catalyzed azide-alkyl cycloaddition (CuAAC)) between the azide group of Compound 3 and the alkyne group of Compound 4. Abbreviations: Cu, copper; and THPTA, tris(3-hydroxypropyltriazolylmethyl)amine.

FIG. 10 shows a route for the synthesis of an azide-functionalized, heparanase-sensitive (HS) heparin oligomer, referred to herein as Compound 1, which is a synthetic intermediate in the synthesis of a HS oligosaccharide conjugate i.e. Compound 5. Abbreviations: pmHS2, Pasteurella multocida heparosan synthase-2; UDP-GlcNTFA, uridine diphospho N-trifluoroacetyl glucosamine; UDP-GlcA, uridine diphospho glucuronic acid; UDP-GlcNAc, uridine diphospho N-acetylglucosamine; 2-OST, 2-O-sulfotransferase; 6-OST, 6-O-sulfotransferase 3; NST, N-sulfotransferase; PAPS, 3′-phosphoadenosine 5′-phosphosulfate; 3-OST, 3-O-sulfotransferase; M, molar; and LiOH, lithium hydroxide.

FIG. 11 shows a route for the synthesis of an alkyne-functionalized oligosaccharide-containing compound, referred to herein as Compound 2, a synthetic intermediate in the synthesis of heparanase-sensitive (HS) oligosaccharide conjugate, i.e., Compound 5. Abbreviations: pmHS2, Pasteurella multocida heparosan synthase-2; UDP-GlcNTFA, uridine diphospho N-trifluoroacetyl glucosamine; UDP-GlcA, uridine diphospho glucuronic acid; NST, N-sulfotransferase; 6-OST, 6-O-sulfotransferase 3; PAPS, 3′-phosphoadenosine 5′-phosphosulfate; H₂, hydrogen gas; Pd, palladium; M, molar; and LiOH, lithium hydroxide.

FIG. 12 shows a route for the synthesis of heparanase-sensitive (HR) oligosaccharide conjugate, referred to herein as Compound 5, from Compound 1 and Compound 2 via an exemplary Click chemistry reaction (more particularly, a copper-catalyzed azide-alkyl cycloaddition (CuAAC)) between the azide group of Compound 1 and the alkyne group of Compound 2. Abbreviations: Cu, copper; and THPTA, tris(3-hydroxypropyltriazolylmethyl)amine.

FIGS. 13A and 13B compare the anticoagulant activities of heparanase-resistant (HR) oligosaccharide conjugate Compound 6, heparanase-sensitive (HS) oligosaccharide conjugate Compound 5, and Compound 3, a synthetic intermediate of Compound 6. FIG. 13A shows the structures of (top) Compound 3, (middle) Compound 6, and (bottom) Compound 5. The heparin heptamer (7-mer) oligomer and two galactosamine residues in Compound 6 are shown in boxes. FIG. 13B is a graph showing the anticoagulant activities of Compound 6 (2.4 micrograms per milliliter (μg/mL)), Compound 3 (2.4 μg/mL), and Compound 5 (2.4 μg/mL) evaluated by testing the inhibitory effect of the compounds to Factor IIa (FIIa) both before and after heparanase digestion. The remaining activity of FIIa after treatment with the compound indicated in the x-axis, or the heparanase digested compound, is shown as a percentage (%). Phosphate-buffered saline (PBS) was used as a control. The data are expressed as mean (n=3)±SEM.

FIGS. 14A and 14B are structural analysis of the heparanase digestion of (FIG. 14A) Compound 6 and (FIG. 14B) Compound 5 by liquid chromatography/mass spectrometry (LC/MS). Sites of cleavage and the number of oligosaccharide residues of the digested fragments are indicated.

FIGS. 15A and 15B are graphs showing the high-performance liquid chromatography (HPLC) analysis of the heparanase digestion of Compounds 5 and 6. FIG. 15A shows (left) a graph of the HPLC chromatograms (optical density (O.D.) at 255 nanometers (nm) versus retention time (R. time) in minutes (min)) of Compound 5 (upper chromatogram) before and (lower chromatogram) after heparanase digestion. At right is a graph of the LC chromatogram (intensity versus R. time) pf Compound 5 prior to analysis by mass spectrometry. FIG. 15B shows (left) a graph of the HPLC chromatograms (O.D. at 255 nm versus R. time in min) of Compound 6 (upper chromatogram) before and (lower chromatogram) after heparanase digestion. At right is a graph of the LC chromatogram (intensity versus R. time) of Compound 6 prior to analysis by mass spectrometry.

### DETAILED DESCRIPTION

Provided herein are heparanase-resistant (HR), ultralow molecular weight heparin compounds that do not contain an internal GlcA residue, but otherwise display potent anticoagulant activity. A chemoenzymatic scheme was developed using a glycosyl transferase (pmHS2), an epimerase (C5-epi), and four distinct sulfotransferases, including NST, 2-OST, 3-OST-3 and 6-OSTs, which replaced -[GlcA-GlcNS3S6S]- with -[IdoA2S-GlcNS3S6S]—. For instance, provided herein is a heparin oligosaccharide that displays nanomolar anti-FXa activity, yet is resistant to heparanase digestion. Such compounds inhibit thrombus formation, for example, after subcutaneous administration in a murine model of venous thrombosis.

In one aspect, provided herein is a heparin oligomer of Formula (I).

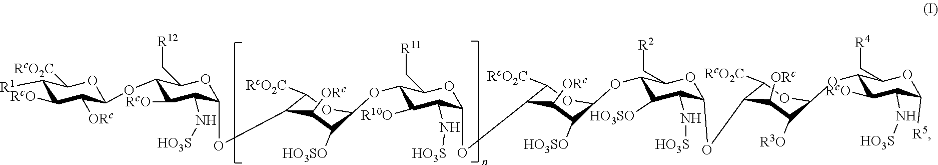

or a pharmaceutically acceptable salt, solvate, hydrate, polymorph, co-crystal, tautomer, stereoisomer, isotopically labeled derivative, or prodrug thereof, wherein n, R¹, R², R³, R⁴, R⁵, R¹⁰, R¹¹, R¹² and R^{C} are as defined herein.

In another aspect, the present disclosure provides oligomeric compounds comprising two or more repeat units connected via a linker, and pharmaceutically acceptable salts, solvates, hydrates, polymorphs, co-crystals, tautomers, stereoisomers, isotopically labeled derivatives, and prodrugs thereof, wherein the repeat units are each independently a heparin oligomer provided herein.

In another aspect, the present disclosure provides polymer conjugates, and pharmaceutically acceptable salts, solvates, hydrates, polymorphs, co-crystals, tautomers, stereoisomers, isotopically labeled derivatives, and prodrugs thereof, wherein the polymer conjugate comprises a heparin oligomer or oligomeric compound provided herein, or a pharmaceutically acceptable salt, solvate, hydrate, polymorph, co-crystal, tautomer, stereoisomer, isotopically labeled derivative, or prodrug thereof, conjugated to a polymer via a linker.

In another aspect, the present disclosure provides oligosaccharide conjugates, and pharmaceutically acceptable salts, solvates, hydrates, polymorphs, co-crystals, tautomers, stereoisomers, isotopically labeled derivatives, and prodrugs thereof, wherein the oligosaccharide conjugate comprises a domain comprising a heparin oligomer or oligomeric compound provided herein, or a pharmaceutically acceptable salt, solvate, hydrate, polymorph, co-crystal, tautomer, stereoisomer, isotopically labeled derivative, or prodrug thereof, conjugated to a second domain comprising an oligosaccharide-containing oligomer via a linker.

In another aspect, the present disclosure provides pharmaceutical compositions comprising a heparin oligomer, oligomeric compound, polymer conjugate, or oligosaccharide conjugate as provided herein, or a pharmaceutically acceptable salt, solvate, hydrate, polymorph, co-crystal, tautomer, stereoisomer, isotopically labeled derivative, or prodrug thereof, and a pharmaceutically acceptable excipient.

In another aspect, the present disclosure provides kits comprising: a heparin oligomer, oligomeric compound, polymer conjugate, or oligosaccharide conjugate as provided herein, or a pharmaceutically acceptable salt, solvate, hydrate, polymorph, co-crystal, tautomer, stereoisomer, isotopically labeled derivative, or prodrug thereof, or a pharmaceutical composition provided herein; and instructions for administering to a subject the heparin oligomer, oligomeric compound, polymer conjugate, oligosaccharide conjugate or the pharmaceutical composition.

In another aspect, the present disclosure provides surface coatings comprising a heparin oligomer, oligomeric compound, polymer conjugate, or oligosaccharide conjugate provided herein, or a pharmaceutically acceptable salt, solvate, hydrate, polymorph, co-crystal, tautomer, stereoisomer, isotopically labeled derivative, or prodrug thereof, and an excipient.

In another aspect, the present disclosure provides devices comprising a surface coating provided herein.

In another aspect, the present disclosure provides methods of treating or preventing a disease in a subject in need thereof, comprising administering to the subject an effective amount of a heparin oligomer, oligomeric compound, polymer conjugate, or oligosaccharide conjugate provided herein, or a pharmaceutically acceptable salt, solvate, hydrate, polymorph, co-crystal, tautomer, stereoisomer, isotopically labeled derivative, or prodrug thereof, or a pharmaceutical composition provided herein.

In another aspect, the present disclosure provides a use of a heparin oligomer, oligomeric compound, polymer conjugate, or oligosaccharide conjugate provided herein, or a pharmaceutically acceptable salt, solvate, hydrate, polymorph, co-crystal, tautomer, stereoisomer, isotopically labeled derivative, or prodrug thereof, or a pharmaceutical composition provided herein, for the manufacture of a medicament for treating or preventing a disease in a subject in need thereof.

In another aspect, the present disclosure provides a heparin oligomer, an oligomeric compound, polymer conjugate, or oligosaccharide conjugate, or a pharmaceutically acceptable salt, solvate, hydrate, polymorph, co-crystal, tautomer, stereoisomer, isotopically labeled derivative, or prodrug thereof, or a pharmaceutical composition for use in treating or preventing a disease in a subject in need thereof.

In another aspect, the disclosure provides methods of synthesizing a heparin oligomer (e.g., a heparin heptamer) provided herein.

The presently disclosed subject matter now will be described more fully hereinafter, in which some, but not all embodiments of the presently disclosed subject matter are described. Indeed, the disclosed subject matter can be embodied in many different forms and should not be construed as limited to the embodiments set forth herein; rather, these embodiments are provided so that this disclosure will satisfy applicable legal requirements.

### I. DEFINITIONS

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modem Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers. Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “isotopes” refers to variants of a particular chemical element such that, while all isotopes of a given element share the same number of protons in each atom of the element, those isotopes differ in the number of neutrons.

When a range of values is listed, it is intended to encompass each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylindenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

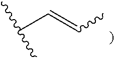

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃—₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, =NNR^{bb}C(═O)R^{aa}, =NNR^{bb}C(═O)OR^{aa}, =NNR^{bb}S(═O)₂R^{aa}, ═NR^{bb}, or ═NOR^{cc}; wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂R^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R_{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂+X⁻, —NH₂(C₁₋₆ alkyl)+X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}—OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and Rad are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}—N(R^{cc})₂, —C(═O)R^{aa}, —C(═O)N(R^{cc})₂, —CO₂R^{aa}—SO₂R^{aa}—C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group comprising formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group comprising methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group comprising p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group comprising phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}—C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}—SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb}) 2)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group comprising methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group comprising —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}—SO₂R^{aa}, —Si(R^{aa})₃, —P(R^{cc})₂, P(R^{c})₃⁺X⁻, —P(OR^{cc})₂, —P(OR^{c})₃⁺X⁻, —P(═O)(R^{aa})₂, —P(═O)(OR^{aa})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

The disclosure is not intended to be limited in any manner by the above exemplary listing of substituents. Additional terms may be defined in other sections of this disclosure.

The term “monosaccharide” refers to a simple form of a sugar that consists of a single saccharide molecule which cannot be further decomposed by hydrolysis. Monosaccharides can be naturally occurring or synthesized. Most monosaccharides exist as either ring-opened monosaccharides or cyclic monosaccharides. Monosaccharides include, but are not limited to, trioses, such as glycerose and dihydroxyacetone; textroses such as erythrose and erythrulose; pentoses such as xylose, arabinose, ribose, xylulose ribulose; methyl pentoses (6-deoxyhexoses), such as rhamnose and fucose; hexoses, such as glucose, mannose, galactose, fructose and sorbose; and heptoses, such as glucoheptose, galamannoheptose, sedoheptulose and mannoheptulose.

As used herein, the term “residue” refers to a bivalent moiety derived from a monosaccharide unit that forms part of an oligosaccharide or polysaccharide. Thus, for example, the residue can be a bivalent moiety derived from a monosaccharide unit by loss of the anomeric hydroxyl group and a H atom of another hydroxyl group.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C1-C8 alkyl, C2-C8 alkenyl, C2-C8 alkynyl, aryl, C7-C12 substituted aryl, and C7-C12 arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “tissue” refers to any biological tissue of a subject (including a group of cells, a body part, or an organ) or a part thereof, including blood and/or lymph vessels A tissue may be an abnormal or unhealthy tissue, which may need to be treated. A tissue may also be a normal or healthy tissue that is under a higher than normal risk of becoming abnormal or unhealthy, which may need to be prevented.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the disclosure may be administered at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for reducing thrombus formation. In certain embodiments, a therapeutically effective amount is an amount sufficient for inhibiting thrombus formation. In certain embodiments, a therapeutically effective amount is an amount sufficient for treating a disease. In certain embodiments, a therapeutically effective amount is an amount sufficient for treating cardiovascular disease, atherosclerosis, atherosclerotic lesions, thrombus formation, thromboembolism, stroke, myocardial infarction, or primary or recurrent venous thromboembolism (VTE). In certain embodiments, a therapeutically effective amount is an amount sufficient for reducing thrombus formation and treating a disease. In certain embodiments, a therapeutically effective amount is an amount sufficient for inhibiting thrombus formation and treating a disease. In certain embodiments, a therapeutically effective amount is an amount sufficient for reducing thrombus formation and treating cardiovascular disease, atherosclerosis, atherosclerotic lesions, thrombus formation, thromboembolism, stroke, myocardial infarction, or primary or recurrent venous thromboembolism (VTE). In certain embodiments, a therapeutically effective amount is an amount sufficient for inhibiting thrombus formation and treating cardiovascular disease, atherosclerosis, atherosclerotic lesions, thrombus formation, thromboembolism, stroke, myocardial infarction, or primary or recurrent venous thromboembolism (VTE).

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for reducing thrombus formation. In certain embodiments, a prophylactically effective amount is an amount sufficient for inhibiting thrombus formation. In certain embodiments, a prophylactically effective amount is an amount sufficient for preventing a disease. In certain embodiments, a prophylactically effective amount is an amount sufficient for preventing cardiovascular disease, atherosclerosis, atherosclerotic lesions, thrombus formation, thromboembolism, stroke, myocardial infarction, or primary or recurrent venous thromboembolism (VTE). In certain embodiments, a prophylactically effective amount is an amount sufficient for reducing thrombus formation and preventing a disease. In certain embodiments, a prophylactically effective amount is an amount sufficient for inhibiting thrombus formation and preventing a disease. In certain embodiments, a prophylactically effective amount is an amount sufficient for reducing thrombus formation and preventing cardiovascular disease, atherosclerosis, atherosclerotic lesions, thrombus formation, thromboembolism, stroke, myocardial infarction, or primary or recurrent venous thromboembolism (VTE). In certain embodiments, a prophylactically effective amount is an amount sufficient for inhibiting thrombus formation and preventing cardiovascular disease, atherosclerosis, atherosclerotic lesions, thrombus formation, thromboembolism, stroke, myocardial infarction, or primary or recurrent venous thromboembolism (VTE).

The term “cardiovascular disease” refers to diseases and disorders of the heart and circulatory system. Exemplary cardiovascular diseases, including cholesterol- or lipid-related disorders, include, but are not limited to acute coronary syndrome, angina, arrhythmia, arteriosclerosis, atherosclerosis, atherosclerotic lesions, carotid atherosclerosis, cerebrovascular disease, cerebral infarction, congestive heart failure, congenital heart disease, coronary heart disease, coronary artery disease, coronary plaque stabilization, dyslipidemias, dyslipoproteinemias, endothelium dysfunctions, familial hypercholeasterolemia, familial combined hyperlipidemia, hypoalphalipoproteinemia, hypertriglyceridemia, hyperbetalipoproteinemia, hypercholesterolemia, hypertension, hyperlipidemia, intermittent claudication, ischemia, ischemia reperfusion injury, ischemic heart diseases, cardiac ischemia, metabolic syndrome, multi-infarct dementia, myocardial infarction, obesity, peripheral vascular disease, reperfusion injury, restenosis, renal artery atherosclerosis, rheumatic heart disease, stroke, thrombotic disorder, thrombus formation, thromboembolism, primary or recurrent venous thromboembolism (VTE), deep vein thrombosis, pulmonary embolism, non-occlusive venous thrombosis, transitory ischemic attacks, and lipoprotein abnormalities associated with Alzheimer's disease, obesity, diabetes mellitus, syndrome X, impotence, multiple sclerosis, Parkinson's diseases and inflammatory diseases.

The term “kidney disease” refers to a disorder of at least one kidney in a human, wherein the disorder compromises or impairs the function of the kidney(s). In some embodiments, kidney disease is characterized physiologically by the leakage of protein into the urine, or by the excretion of nitrogenous waste. In some embodiments, kidney disease results from a primary pathology of the kidney, such as injury to the glomerulus or tubule, or from damage to another organ, such as the pancreas, which adversely affects the ability of the kidney to perform biological functions, such as the retention of protein. Thus, kidney disease in the human can be the direct or indirect effect of a disease condition which may affect other organs. Exemplary kidney diseases include Abderhalden-Kaufmann-Lignac syndrome (Nephropathic Cystinosis), Abdominal Compartment Syndrome, Acetaminophen-induced Nephrotoxicity, Acute Kidney Failure/Acute Kidney Injury, Acute Lobar Nephronia, Acute Phosphate Nephropathy, Acute Tubular Necrosis, Adenine Phosphoribosyltransferase Deficiency, Adenovirus Nephritis, Alagille Syndrome, Alport Syndrome, Amyloidosis, ANCA Vasculitis Related to Endocarditis and Other Infections, Angiomyolipoma, Analgesic Nephropathy, Anorexia Nervosa and Kidney Disease, Angiotensin Antibodies and Focal Segmental Glomerulosclerosis, Antiphospholipid Syndrome, Anti-TNF-α Therapy-related Glomerulonephritis, APOL1 Mutations, Apparent Mineralocorticoid Excess Syndrome, Aristolochic Acid Nephropathy, Chinese Herbal Nephropathy, Balkan Endemic Nephropathy, Arteriovenous Malformations and Fistulas of the Urologic Tract, Autosomal Dominant Hypocalcemia, Bardet-Biedl Syndrome, Bartter Syndrome, Bath Salts and Acute Kidney Injury, Beer Potomania, Beeturia, β-Thalassemia Renal Disease, Bile Cast Nephropathy, BK Polyoma Virus Nephropathy in the Native Kidney, Bladder Rupture, Bladder Sphincter Dyssynergia, Bladder Tamponade, Border-Crossers' Nephropathy, Bourbon Virus and Acute Kidney Injury, Burnt Sugarcane Harvesting and Acute Renal Dysfunction, Byetta and Renal Failure, C1q Nephropathy, C3 Glomerulopathy, C3 Glomerulopathy with Monoclonal Gammopathy, C4 Glomerulopathy, Calcineurin Inhibitor Nephrotoxicity, Callilepsis Laureola Poisoning, Cannabinoid Hyperemesis Acute Renal Failure, Cardiorenal syndrome, Carfilzomib-Indiced Renal Injury, CFHR5 nephropathy, Charcot-Marie-Tooth Disease with Glomerulopathy, Chinese Herbal Medicines and Nephrotoxicity, Cherry Concentrate and Acute Kidney Injury, Cholesterol Emboli, Churg-Strauss syndrome, Chyluria, Ciliopathy, Cocaine and the Kidney, Cold Diuresis, Colistin Nephrotoxicity, Collagenofibrotic Glomerulopathy, Collapsing Glomerulopathy, Collapsing Glomerulopathy Related to CMV, Combination Antiretroviral (cART) Related-Nephropathy, Congenital Anomalies of the Kidney and Urinary Tract (CAKUT), Congenital Nephrotic Syndrome, Congestive Renal Failure, Conorenal syndrome (Mainzer-Saldino Syndrome or Saldino-Mainzer Disease), Contrast Nephropathy, Copper Sulphate Intoxication, Cortical Necrosis, Crizotinib-related Acute Kidney Injury, Cryocrystalglobulinemia, Cryoglobuinemia, Crystalglobulin-Induced Nephropathy, Crystal-Induced Acute Kidney injury, Crystal-Storing Histiocytosis, Cystic Kidney Disease, Acquired, Cystinuria, Dasatinib-Induced Nephrotic-Range Proteinuria, Dense Deposit Disease (MPGN Type 2), Dent Disease (X-linked Recessive Nephrolithiasis), DHA Crystalline Nephropathy, Dialysis Disequilibrium Syndrome, Diabetes and Diabetic Kidney Disease, Diabetes Insipidus, Dietary Supplements and Renal Failure, Diffuse Mesangial Sclerosis, Diuresis, Djenkol Bean Poisoning (Djenkolism), Down Syndrome and Kidney Disease, Drugs of Abuse and Kidney Disease, Duplicated Ureter, EAST syndrome, Ebola and the Kidney, Ectopic Kidney, Ectopic Ureter, Edema, Swelling, Erdheim-Chester Disease, Fabry's Disease, Familial Hypocalciuric Hypercalcemia, Fanconi Syndrome, Fraser syndrome, Fibronectin Glomerulopathy, Fibrillary Glomerulonephritis and Immunotactoid Glomerulopathy, Fraley syndrome, Fluid Overload, Hypervolemia, Focal Segmental Glomerulosclerosis, Focal Sclerosis, Focal Glomerulosclerosis, Galloway Mowat syndrome, Giant Cell (Temporal) Arteritis with Kidney Involvement, Gestational Hypertension, Gitelman Syndrome, Glomerular Diseases, Glomerular Tubular Reflux, Glycosuria, Goodpasture Syndrome, Green Smoothie Cleanse Nephropathy, HANAC Syndrome, Harvoni (Ledipasvir with Sofosbuvir)-Induced Renal Injury, Hair Dye Ingestion and Acute Kidney Injury, Hantavirus Infection Podocytopathy, Heat Stress Nephropathy, Hematuria (Blood in Urine), Hemolytic Uremic Syndrome (HUS), Atypical Hemolytic Uremic Syndrome (aHUS), Hemophagocytic Syndrome, Hemorrhagic Cystitis, Hemorrhagic Fever with Renal Syndrome (HFRS, Hantavirus Renal Disease, Korean Hemorrhagic Fever, Epidemic Hemorrhagic Fever, Nephropathis Epidemica), Hemosiderinuria, Hemosiderosis related to Paroxysmal Nocturnal Hemoglobinuria and Hemolytic Anemia, Hepatic Glomerulopathy, Hepatic Veno-Occlusive Disease, Sinusoidal Obstruction Syndrome, Hepatitis C-Associated Renal Disease, Hepatocyte Nuclear Factor 10-Associated Kidney Disease, Hepatorenal Syndrome, Herbal Supplements and Kidney Disease, High Altitude Renal Syndrome, High Blood Pressure and Kidney Disease, HIV-Associated Immune Complex Kidney Disease (HIVICK), HIV-Associated Nephropathy (HIVAN), HNF1B-related Autosomal Dominant Tubulointerstitial Kidney Disease, Horseshoe Kidney (Renal Fusion), Hunner's Ulcer, Hydroxychloroquine-induced Renal Phospholipidosis, Hyperaldosteronism, Hypercalcemia, Hyperkalemia, Hypermagnesemia, Hypernatremia, Hyperoxaluria, Hyperphosphatemia, Hypocalcemia, Hypocomplementemic Urticarial Vasculitic Syndrome, Hypokalemia, Hypokalemia-induced renal dysfunction, Hypokalemic Periodic Paralysis, Hypomagnesemia, Hyponatremia, Hypophosphatemia, Hypophosphatemia in Users of Cannabis, Hypertension, Hypertension, Monogenic, Iced Tea Nephropathy, Ifosfamide Nephrotoxicity, IgA Nephropathy, IgG4 Nephropathy, Immersion Diuresis, Immune-Checkpoint Therapy-Related Interstitial Nephritis, Infliximab-Related Renal Disease, Interstitial Cystitis, Painful Bladder Syndrome (Questionnaire), Interstitial Nephritis, Interstitial Nephritis, Karyomegalic, Ivemark's syndrome, JC Virus Nephropathy, Joubert Syndrome, Ketamine-Associated Bladder Dysfunction, Kidney Stones, Nephrolithiasis, Kombucha Tea Toxicity, Lead Nephropathy and Lead-Related Nephrotoxicity, Lecithin Cholesterol Acyltransferase Deficiency (LCAT Deficiency), Leptospirosis Renal Disease, Light Chain Deposition Disease, Monoclonal Immunoglobulin Deposition Disease, Light Chain Proximal Tubulopathy, Liddle Syndrome, Lightwood-Albright Syndrome, Lipoprotein Glomerulopathy, Lithium Nephrotoxicity, LMX1B Mutations Cause Hereditary FSGS, Loin Pain Hematuria, Lupus, Systemic Lupus Erythematosis, Lupus Kidney Disease, Lupus Nephritis, Lupus Nephritis with Antineutrophil Cytoplasmic Antibody Seropositivity, Lupus Podocytopathy, Lyme Disease-Associated Glomerulonephritis, Lysinuric Protein Intolerance, Lysozyme Nephropathy, Malarial Nephropathy, Malignancy-Associated Renal Disease, Malignant Hypertension, Malakoplakia, McKittrick-Wheelock Syndrome, MDMA (Molly; Ecstacy; 3,4-Methylenedioxymethamphetamine) and Kidney Failure, Meatal Stenosis, Medullary Cystic Kidney Disease, Urolodulin-Associated Nephropathy, Juvenile Hyperuricemic Nephropathy Type 1, Medullary Sponge Kidney, Megaureter, Melamine Toxicity and the Kidney, MELAS Syndrome, Membranoproliferative Glomerulonephritis, Membranous Nephropathy, Membranous-like Glomerulopathy with Masked IgG Kappa Deposits, MesoAmerican Nephropathy, Metabolic Acidosis, Metabolic Alkalosis, Methotrexate-related Renal Failure, Microscopic Polyangiitis, Milk-alkalai syndrome, Minimal Change Disease, Monoclonal Gammopathy of Renal Significance, Dysproteinemia, Mouthwash Toxicity, MUC1 Nephropathy, Multicystic dysplastic kidney, Multiple Myeloma, Myeloproliferative Neoplasms and Glomerulopathy, Nail-patella Syndrome, NARP Syndrome, Nephrocalcinosis, Nephrogenic Systemic Fibrosis, Nephroptosis (Floating Kidney, Renal Ptosis), Nephrotic Syndrome, Neurogenic Bladder, 9/11 and Kidney Disease, Nodular Glomerulosclerosis, Non-Gonococcal Urethritis, Nutcracker syndrome, Oligomeganephronia, Orofaciodigital Syndrome, Orotic Aciduria, Orthostatic Hypotension, Orthostatic Proteinuria, Osmotic Diuresis, Osmotic Nephrosis, Ovarian Hyperstimulation Syndrome, Oxalate Nephropathy, Page Kidney, Papillary Necrosis, Papillorenal Syndrome (Renal-Coloboma Syndrome, Isolated Renal Hypoplasia), PARN Mutations and Kidney Disease, Parvovirus B19 and the Kidney, The Peritoneal-Renal Syndrome, POEMS Syndrome, Posterior Urethral Valve, Podocyte Infolding Glomerulopathy, Post-infectious Glomerulonephritis, Post-streptococcal Glomerulonephritis, Post-infectious Glomerulonephritis, Atypical, Post-Infectious Glomerulonephritis (IgA-Dominant), Mimicking IgA Nephropathy, Polyarteritis Nodosa, Polycystic Kidney Disease, Posterior Urethral Valves, Post-Obstructive Diuresis, Preeclampsia, Propofol infusion syndrome, Proliferative Glomerulonephritis with Monoclonal IgG Deposits (Nasr Disease), Propolis (Honeybee Resin) Related Renal Failure, Proteinuria (Protein in Urine), Pseudohyperaldosteronism, Pseudohypobicarbonatemia, Pseudohypoparathyroidism, Pulmonary-Renal Syndrome, Pyelonephritis (Kidney Infection), Pyonephrosis, Pyridium and Kidney Failure, Radiation Nephropathy, Ranolazine and the Kidney, Refeeding syndrome, Reflux Nephropathy, Rapidly Progressive Glomerulonephritis, Renal Abscess, Peripnephric Abscess, Renal Agenesis, Renal Arcuate Vein Microthrombi-Associated Acute Kidney Injury, Renal Artery Aneurysm, Renal Artery Dissection, Spontaneous, Renal Artery Stenosis, Renal Cell Cancer, Renal Cyst, Renal Hypouricemia with Exercise-induced Acute Renal Failure, Renal Infarction, Renal Osteodystrophy, Renal Tubular Acidosis, Renin Mutations and Autosomal Dominant Tubulointerstitial Kidney Disease, Renin Secreting Tumors (Juxtaglomerular Cell Tumor), Reset Osmostat, Retrocaval Ureter, Retroperitoneal Fibrosis, Rhabdomyolysis, Rhabdomyolysis related to Bariatric Surgery, Rheumatoid Arthritis-Associated Renal Disease, Sarcoidosis Renal Disease, Salt Wasting, Renal and Cerebral, Schistosomiasis and Glomerular Disease, Schimke immuno-osseous dysplasia, Scleroderma Renal Crisis, Serpentine Fibula-Polycystic Kidney Syndrome, Exner Syndrome, Sickle Cell Nephropathy, Silica Exposure and Chronic Kidney Disease, Sri Lankan Farmers' Kidney Disease, Sjogren's Syndrome and Renal Disease, Synthetic Cannabinoid Use and Acute Kidney Injury, Kidney Disease Following Hematopoietic Cell Transplantation, Kidney Disease Related to Stem Cell Transplantation, TAFRO Syndrome, Tea and Toast Hyponatremia, Tenofovir-Induced Nephrotoxicity, Thin Basement Membrane Disease, Benign Familial Hematuria, Thrombotic Microangiopathy Associated with Monoclonal Gammopathy, Trench Nephritis, Trigonitis, Tuberculosis, Genitourinary, Tuberous Sclerosis, Tubular Dysgenesis, Immune Complex Tubulointerstitial Nephritis Due to Autoantibodies to the Proximal Tubule Brush Border, Tumor Lysis Syndrome, Uremia, Uremic Optic Neuropathy, Ureteritis Cystica, Ureterocele, Urethral Caruncle, Urethral Stricture, Urinary Incontinence, Urinary Tract Infection, Urinary Tract Obstruction, Urogenital Fistula, Uromodulin-Associated Kidney Disease, Vancomycin-Associated Cast Nephropathy, Vasomotor Nephropathy, Vesicointestinal Fistula, Vesicoureteral Reflux, VGEF Inhibition and Renal Thrombotic Microangiopathy, Volatile Anesthetics and Acute Kidney Injury, Von Hippel-Lindau Disease, Waldenstrom's Macroglobulinemic Glomerulonephritis, Warfarin-Related Nephropathy, Wasp Stings and Acute Kidney Injury, Wegener's Granulomatosis, Granulomatosis with Polyangiitis, West Nile Virus and Chronic Kidney Disease, Wunderlich syndrome, Zellweger Syndrome, or Cerebrohepatorenal Syndrome.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

A “diabetic condition” refers to diabetes and pre-diabetes. Diabetes refers to a group of metabolic diseases in which a person has high blood sugar, either because the body does not produce enough insulin, or because cells do not respond to the insulin that is produced. This high blood sugar produces the classical symptoms of polyuria (frequent urination), polydipsia (increased thirst) and polyphagia (increased hunger). There are several types of diabetes. Type I diabetes results from the body's failure to produce insulin, and presently requires the person to inject insulin or wear an insulin pump. Type II diabetes results from insulin resistance a condition in which cells fail to use insulin properly, sometimes combined with an absolute insulin deficiency. Gestational diabetes occurs when pregnant women without a previous diagnosis of diabetes develop a high blood glucose level. Other forms of diabetes include congenital diabetes, which is due to genetic defects of insulin secretion, cystic fibrosis-related diabetes, steroid diabetes induced by high doses of glucocorticoids, and several forms of monogenic diabetes, e.g., mature onset diabetes of the young (e.g., MODY 1, 2, 3, 4, 5, 6, 7, 8, 9, or 10). Pre-diabetes indicates a condition that occurs when a person's blood glucose levels are higher than normal but not high enough for a diagnosis of diabetes. All forms of diabetes increase the risk of long-term complications. These typically develop after many years, but may be the first symptom in those who have otherwise not received a diagnosis before that time. The major long-term complications relate to damage to blood vessels. Diabetes doubles the risk of cardiovascular disease and macrovascular diseases such as ischemic heart disease (angina, myocardial infarction), stroke, and peripheral vascular disease. Diabetes also causes microvascular complications, e.g., damage to the small blood vessels. Diabetic retinopathy, which affects blood vessel formation in the retina of the eye, can lead to visual symptoms, reduced vision, and potentially blindness. Diabetic nephropathy, the impact of diabetes on the kidneys, can lead to scarring changes in the kidney tissue, loss of small or progressively larger amounts of protein in the urine, and eventually chronic kidney disease requiring dialysis. Diabetic neuropathy is the impact of diabetes on the nervous system, most commonly causing numbness, tingling and pain in the feet and also increasing the risk of skin damage due to altered sensation. Together with vascular disease in the legs, neuropathy contributes to the risk of diabetes-related foot problems, e.g., diabetic foot ulcers, that can be difficult to treat and occasionally require amputation.

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds disclosed herein may also be useful in treating inflammation associated with cancer.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “metastatic disease” refers to conditions which can spread to another organ or tissue to another non-adjacent organ or tissue. In some embodiments, the metastatic disease refers to a metastatic cancer disease. Metastatic diseases include, but are not limited to, metastatic cancer spread derived from a carcinoma, a sarcoma, a lymphoma, a leukemia, a germ cell tumor, and/or a blastoma. Metastatic diseases also include metastatic spread from benign tumors. In some embodiments, the metastatic disease further includes metastatic spread from cancerous or benign tumors of the bladder, the colon, the liver, the lung, the breast, the vagina, the ovaries, the pancreas, the kidney, the stomach, gastrointestinal tract, the prostate, the head and neck, the peritoneal cavity, the thyroid, the bone, the brain, the central nervous system, the blood, and/or melanoma.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Unless otherwise required by context, singular terms shall include pluralities, and plural terms shall include the singular.

### II. EXEMPLARY EMBODIMENTS

Provided herein are heparin oligomers (e.g., heparin oligomers of Formula (I)), pharmaceutically acceptable salts, solvates, hydrates, polymorphs, co-crystals, tautomers, stereoisomers, isotopically labeled derivatives, and prodrugs thereof, oligomers, polymer conjugates, oligosaccharide conjugates, pharmaceutical compositions, surface coatings, devices, kits, methods, and uses. The disclosure seeks to improve reduction or inhibition of thrombus formation by using heparin oligomers (e.g., of Formula (I)).

### Heparin Oligomers

In one aspect, provided herein is a heparin oligomer having a structure of Formula (I):

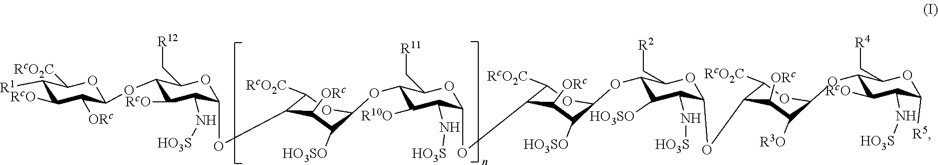

or a pharmaceutically acceptable salt, solvate, hydrate, polymorph, co-crystal, tautomer, stereoisomer, isotopically labeled derivative, or prodrug thereof, wherein: n is 0, 1, 2, 3, 4, 5, 6, 7, 8, 9, or 10; R¹ is —OR^{A}, —SR^{A}, —N(R^{A})₂, halogen, an optionally substituted monosaccharide, or an optionally substituted oligosaccharide; each occurrence of R^{A} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atoms to form an optionally substituted heterocyclic ring or optionally substituted heteroaryl ring; each of R², R⁴, R¹¹, and R¹² is independently —H, —OH, —OSO₃H, or —SO₃H; each of R³ and R¹⁰ is independently —H, —SO₃H, optionally substituted C₁-C₆ alkyl, or an oxygen protecting group; and R is —OR^{B}, —SR^{B}, —N(R^{B})₂, halogen, an optionally substituted monosaccharide, or an optionally substituted oligosaccharide; each occurrence of R^{B} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{B} are joined together with their intervening atoms to form an optionally substituted heterocyclic ring or optionally substituted heteroaryl ring; and each occurrence of RC is independently —H, optionally substituted C₁-C₆ alkyl, or an oxygen protecting group; provided that one of R¹ and R⁵ is an optionally substituted monosaccharide. Thus, the heparin oligomer is at least a heptamer (i.e., a compound comprising at least 7 saccharide residues).

As defined herein, n is 0, 1, 2, 3, 4, 5, 6, 7, 8, 9, or 10. In some embodiments, n is 0, 1, 2, 3, 4, 5, 6, 7, 8, or 9. In some embodiments, n is 0, 1, 2, 3, 4, 5, 6, 7, or 8. In some embodiments, n is 0, 1, 2, 3, 4, 5, 6, or 7. In some embodiments, n is 0, 1, 2, 3, 4, 5, or 6. In some embodiments, n is 0, 1, 2, 3, 4, or 5. In some embodiments, n is 0, 1, 2, 3, or 4. In some embodiments, n is 0, 1, 2, or 3. In some embodiments, n is 0, 1, or 2. In some embodiments, n is 1, 2, 3, 4, 5, 6, 7, 8, 9, or 10. In some embodiments, n is 1, 2, 3, 4, 5, 6, 7, 8, or 9. In some embodiments, n is 1, 2, 3, 4, 5, 6, 7, or 8. In some embodiments, n is 1, 2, 3, 4, 5, 6, or 7. In some embodiments, n is 1, 2, 3, 4, 5, or 6. In some embodiments, n is 1, 2, 3, 4, or 5. In some embodiments, n is 1, 2, 3, or 4. In some embodiments, n is 1, 2, or 3. In some embodiments, n is 0 or 1. In some embodiments, n is 1 or 2. In some embodiments, n is 2 or 3. In some embodiments, n is 3 or 4. In some embodiments, n is 5 or 6. In some embodiments, n is 7 or 8. In some embodiments, n is 9 or 10. In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, the heparin oligomer has a structure of Formula (I-A):

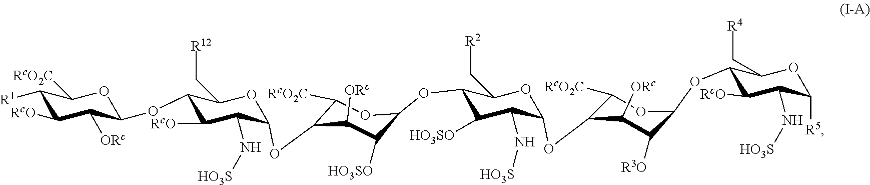

or a pharmaceutically acceptable salt, solvate, hydrate, polymorph, co-crystal, tautomer, stereoisomer, isotopically labeled derivative, or prodrug thereof, wherein: R¹ is —OR^{A}, —SR^{A}, —N(R^{A})₂, halogen, an optionally substituted monosaccharide, or an optionally substituted oligosaccharide; each occurrence of R^{A} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atoms to form an optionally substituted heterocyclic ring or optionally substituted heteroaryl ring; each of R², R⁴, and R¹² is independently —H, —OH, —OSO₃H, or —SO₃H; R³ is —H, —SO₃H, optionally substituted C₁-C₆ alkyl, or an oxygen protecting group; and R⁵ is —OR^{B}, —SR^{B}, —N(R^{B})₂, halogen, or an optionally substituted monosaccharide; each occurrence of R^{B} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{B} are joined together with their intervening atoms to form an optionally substituted heterocyclic ring or optionally substituted heteroaryl ring;
each occurrence of R^{C} is independently —H, optionally substituted C₁-C₆ alkyl, or an oxygen protecting group;
provided that one of R¹ and R⁵ is an optionally substituted monosaccharide.

As defined herein, R¹ is —OR^{A}, —SR^{A}, —N(R^{A})₂, halogen, an optionally substituted monosaccharide, or an optionally substituted oligosaccharide. In some embodiments, R¹ is —OR^{A}, —SR^{A}, —N(R^{A})₂, halogen, or an optionally substituted monosaccharide. In some embodiments, R¹ is —OR^{A}, —SR^{A}, —N(R^{A})₂, or halogen. In some embodiments, R¹ is an optionally substituted monosaccharide or an optionally substituted oligosaccharide. In some embodiments, R¹ is —OR^{A}, —SR^{A}, or —N(R^{A})₂. In some embodiments, R¹ is —OR^{A} or —SR^{A}. In some embodiments, R¹ is —OR^{A} or —N(R^{A})₂. In some embodiments, R¹ is —SR^{A} or —N(R^{A})₂. In some embodiments, R¹ is —OR^{A}. In some embodiments, R¹ is —OH or —O(oxygen protecting group). In some embodiments, R¹ is —SR^{A}. In some embodiments, R¹ is —SH or —S(sulfur protecting group). In some embodiments, R¹ is —N(R^{A})₂. In some embodiments, R¹ is —NH₂ or —NH(nitrogen protecting group). In some embodiments, R¹ is halogen. In some embodiments, R¹ is —F, —Cl, —Br, or —I. In some embodiments, R¹ is —F, —Cl, or —Br. In some embodiments, R¹ is —F or —Cl. In some embodiments, R¹ is —Cl or —Br. In some embodiments, R¹ is —F. In some embodiments, R¹ is —Cl. In some embodiments, R¹ is —Br. In some embodiments, R¹ is —I. In some embodiments, R¹ is an optionally substituted monosaccharide. In some embodiments, R¹ is an optionally substituted oligosaccharide. In some embodiments, R¹ is optionally substituted glucosamine.

In some embodiments, R¹ is

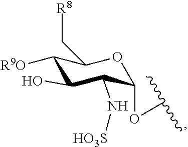

wherein:
R⁸ is —H, —OH, —OSO₃H, or —SO₃H; and
R⁹ is —H, an oxygen protecting group, optionally substituted aliphatic, optionally substituted heteroaliphatic, optionally substituted carbocyclyl, optionally substituted heterocyclyl, optionally substituted aryl, or optionally substituted heteroaryl.

As defined herein, R⁸ is —H, —OSO₃H, or —SO₃H. In some embodiments, R⁸ is —SO₃H or —OSO₃H. In some embodiments, R⁸ is —SO₃H or —H. In some embodiments, R⁸ is —OSO₃H, —OH, or —H. In some embodiments, R⁸ is —OSO₃H. In some embodiments, R⁸ is —H. In some embodiments, R⁸ is —SO₃H. In some embodiments, R⁸ is —OH.

As defined herein, R⁹ is —H, an oxygen protecting group, optionally substituted aliphatic, optionally substituted heteroaliphatic, optionally substituted aryl, or optionally substituted heteroaryl. In some embodiments, R⁹ is —H or an oxygen protecting group. In some embodiments, R⁹ is —H, optionally substituted aliphatic, or optionally substituted heteroaliphatic. In some embodiments, R⁹ is optionally substituted aliphatic or optionally substituted heteroaliphatic. In some embodiments, R⁹ is optionally substituted C₁-C₁₀ aliphatic or optionally substituted C₁-C₁₀ heteroaliphatic. In some embodiments, R⁹ is optionally substituted C₁-C₄ aliphatic or optionally substituted C₁-C₄ heteroaliphatic. In some embodiments, R⁹ is optionally substituted aryl or optionally substituted heteroaryl. In some embodiments, R⁹ is —H. In some embodiments, R⁹ is an oxygen protecting group.

In some embodiments, R⁹ is optionally substituted aliphatic. In some embodiments, R⁹ is optionally substituted C₁-C₁₀ aliphatic. In some embodiments, R⁹ is optionally substituted C₁-C₄ aliphatic. In some embodiments, R⁹ is optionally substituted alkyl. In some embodiments, R⁹ is optionally substituted C₁-C₁₀ alkyl. In some embodiments, R⁹ is optionally substituted C₁-C₄ alkyl. In some embodiments, R⁹ is methyl. In some embodiments, R⁹ is ethyl. In some embodiments, R⁹ is optionally substituted alkenyl. In some embodiments, R⁹ is optionally substituted C₁-C₁₀ alkenyl. In some embodiments, R⁹ is optionally substituted C₁-C₄ alkenyl. In some embodiments, R⁹ is optionally substituted alkynyl. In some embodiments, R⁹ is optionally substituted C₁-C₁₀ alkynyl. In some embodiments, R⁹ is optionally substituted C₁-C₄ alkynyl.

In some embodiments, R⁹ is optionally substituted heteroaliphatic. In some embodiments, R⁹ is optionally substituted C₁-C₁₀ heteroaliphatic. In some embodiments, R⁹ is optionally substituted C₁-C₄ heteroaliphatic. In some embodiments, R⁹ is optionally substituted heteroalkyl. In some embodiments, R⁹ is optionally substituted C₁-C₁₀ heteroalkyl. In some embodiments, R⁹ is optionally substituted C₁-C₄ heteroalkyl. In some embodiments, R⁹ is optionally substituted heteroalkenyl. In some embodiments, R⁹ is optionally substituted C₁-C₁₀ heteroalkenyl. In some embodiments, R⁹ is optionally substituted C₁-C₄ heteroalkenyl. In some embodiments, R⁹ is optionally substituted heteroalkynyl. In some embodiments, R⁹ is optionally substituted C₁-C₁₀ heteroalkynyl. In some embodiments, R⁹ is optionally substituted C₁-C₄ heteroalkynyl.

In some embodiments, R⁹ is optionally substituted carbocyclyl.

In some embodiments, R⁹ is optionally substituted heterocycyl.

In some embodiments, R⁹ is optionally substituted aryl. In some embodiments, R⁹ is optionally substituted monocyclic aryl. In some embodiments, R⁹ is optionally substituted bicyclic aryl. In some embodiments, R⁹ is optionally substituted C₆₋₁₄ aryl. In some embodiments, R⁹ is optionally substituted C₆₋₁₀ aryl. In some embodiments, R⁹ is optionally substituted phenyl. In some embodiments, R⁹ is optionally substituted naphthyl.

In some embodiments, R⁹ is optionally substituted heteroaryl. In some embodiments, R⁹ is optionally substituted monocyclic heteroaryl. In some embodiments, R⁹ is optionally substituted bicyclic heteroaryl. In some embodiments, R⁹ is optionally substituted 5- to 14-membered heteroaryl. In some embodiments, R⁹ is optionally substituted 5- to 10-membered heteroaryl. In some embodiments, R⁹ is optionally substituted 5- to 6-membered monocyclic heteroaryl. In some embodiments, R⁹ is optionally substituted 1- to 10-membered bicyclic heteroaryl.

As defined herein, each occurrence of R^{A} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atoms to form an optionally substituted heterocyclic ring or optionally substituted heteroaryl ring. In some embodiments, each instance of R^{A} is independently —H, optionally substituted alkyl, optionally substituted alkenyl, optionally substituted alkynyl, optionally substituted heteroalkyl, optionally substituted heteroalkenyl, or optionally substituted heteroalkynyl. In some embodiments, each instance of R^{A} is optionally substituted carbocyclyl, optionally substituted heterocyclyl, optionally substituted aryl, or optionally substituted heteroaryl. In some embodiments, each instance of R^{A} is independently —H, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, each instance of R^{A} is independently —H, optionally substituted C₁₋₁₀ alkyl, or optionally substituted phenyl. In some embodiments, R^{A} is —H, a nitrogen protecting group when attached to a nitrogen atom, an oxygen protecting group when attached to an oxygen atom, or a sulfur protecting group when attached to a sulfur atom. In some embodiments, R^{A} is —H. In some embodiments, each instance of R^{A} is a nitrogen protecting group when attached to a nitrogen atom, an oxygen protecting group when attached to an oxygen atom, or a sulfur protecting group when attached to a sulfur atom. In some embodiments, two instances of R^{A} attached to the same intervening atom are joined together with the intervening atom to form an optionally substituted, monocyclic, heterocyclic or heteroaryl ring.

In some embodiments, R¹ is an optionally substituted oligosaccharide. In some embodiments, the optionally substituted oligosaccharide comprises a pentasaccharide moiety with the structure:

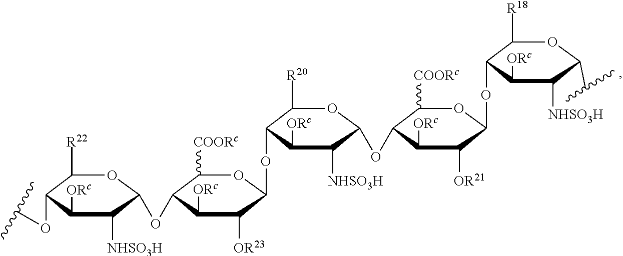

wherein: each of R⁸, R²⁰, and R²² is independently —H, —OH, —OSO₃H, or —SO₃H; each of R² and R²³ is independently —SO₃H, —H, optionally substituted C₁-C₆ alkyl, or an oxygen protecting group; and each occurrence of R^{C} is independently —H, optionally substituted C₁-C₆ alkyl, or an oxygen protecting group.

In some embodiments, R¹ is an optionally substituted monosaccharide or an optionally substituted oligosaccharide having a structure of the formula:

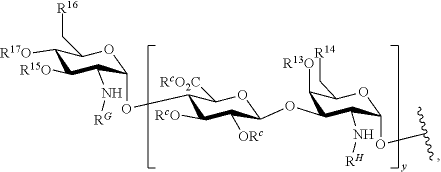

wherein: y is 0, 1, 2, 3, 4, 5, or 6; each of R¹³, R¹⁵, and R¹⁷ is independently —SO₃H, —H, optionally substituted C₁-C₆ alkyl, or an oxygen protecting group; and each R⁴ is independently —OH, an oxygen protecting group, optionally substituted C₁-C₆ alkyl, or —OSO₃H; R¹⁶ is —OH, an oxygen protecting group, optionally substituted C₁-C₆ alkyl, or —OSO₃H; each occurrence of R^{C} is independently —H, optionally substituted C₁-C₆ alkyl, or an oxygen protecting group; R^{H} is optionally substituted acyl; and R^{G} is optionally substituted acyl or optionally substituted alkyl.

In some embodiments, y is 0. In some embodiments, y is 1. In some embodiments, y is 2. In some embodiments, y is 3. In some embodiments, y is 4. In some embodiments, y is 5. In some embodiments, y is 6.

In some embodiments, one or more of R³, R¹⁵, and R¹⁷ is —H. In some embodiments, each of R¹³, R¹⁵, and R¹⁷ is —H.

In some embodiments, each R⁴ is —OH.

In some embodiments, each R¹⁶ is —OH.

In some embodiments, each R^{H} is C(═O)—(C₁-C₆ alkyl). In some embodiments, each R^{H} is —C(═O)CH₃.

In some embodiments, R^{G} is optionally substituted acyl. In some embodiments, R^{G} is optionally substituted alkyl. In some embodiments, the optionally substituted alkyl or optionally substituted acyl is substituted with an azide (—N₃) or a terminal alkyne (—C≡C—H). In some embodiments, R^{G} is C(═O)-(substituted alkyl), wherein substituted alkyl is alkyl substituted with —N₃ or —C≡C—H. In some embodiments, R^{G} is C(═O)-(substituted alkyl), wherein substituted alkyl is C₁-C₆ alkyl (e.g., methyl, ethyl, propyl, butyl, pentyl, or hexyl) substituted with —N₃ or —C≡C—H. In some embodiments, R^{G} is C(═O)-(substituted pentyl), wherein substituted pentyl is pentyl substituted with —N₃ or —C≡C—H.

In some embodiments, R^{G} is optionally substituted alkyl or optionally substituted acyl, wherein the optionally substituted alkyl or optionally substituted acyl is alkyl or acyl substituted with a linker, wherein said linker is covalently bonded to an optionally substituted oligosaccharide. Thus, in some embodiments, R^{G} is attached via the linker to an optionally substitute oligosaccharide. In some embodiments, the linker is —NHC(═O)—, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or any combination thereof.

In some embodiments, the linker is covalently bonded to an optionally substituted oligosaccharide having inhibitory activity against Factor IIa. In some embodiments, the linker is covalently bonded to an optionally substituted oligosaccharide having a structure of Formula (II):

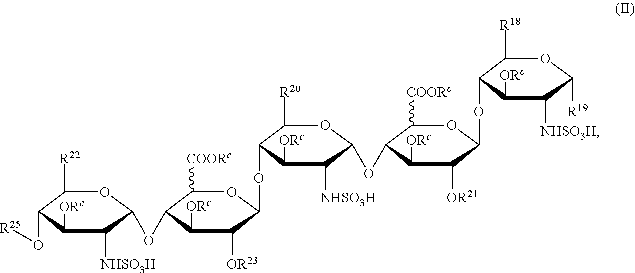

wherein: each of R¹⁸, R²⁰, and R²² is independently —H, —OH, —OSO₃H, or —SO₃H; each of R² and R²³ is independently —SO₃H, —H, optionally substituted C₁-C₆ alkyl, or an oxygen protecting group; each occurrence of R^{C} is independently —H, optionally substituted C₁-C₆ alkyl, or an oxygen protecting group; R¹⁹ is a covalent bond to the linker or a bivalent group covalently bonded to the linker, wherein the bivalent group covalently bonded to the linker is selected from —O—R²⁶—, —SR²⁶—, —N(R^{B})(R²⁶), an optionally substituted monosaccharide residue covalently bonded to the linker, and an optionally substituted oligosaccharide residue covalently bonded to the linker; R²⁶ is a covalent bond to the linker, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene; R^{B} is —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group when attached to a nitrogen atom; or wherein R^{B} and R²⁶ together with the nitrogen atom to which they are attached form an optionally substituted heterocyclic ring or optionally substituted heteroaryl ring that comprises a covalent bond to the linker; and R²⁵ is —H, optionally substituted alkyl, optionally substituted aralkyl, optionally substituted aryl, an optionally substituted monosaccharide, or an optionally substituted oligosaccharide. In some embodiments, R²⁵ comprises one or more optionally substituted galactosamine residues, such as, but not limited to an N- and/or O-sulfated galactosamine residue.

As defined herein, in some embodiments, each of R², R⁴, R¹, and R¹² is independently —H, —OH, —OSO₃H, or —SO₃H. In some embodiments, each of R², R⁴, R¹, and R¹² is independently —H, —OH, or —OSO₃H. In some embodiments, each of R², R⁴, R¹¹, and R¹² is independently —H or —SO₃H. In some embodiments, each of R², R⁴, R¹¹, and R¹² is independently —OH or —OSO₃H. In some embodiments, each of R², R⁴, R¹¹, and R¹² is independently —OSO₃H or —SO₃H.

In some embodiments, R² is —SO₃H or —OSO₃H. In some embodiments, R² is —SO₃H or —H. In some embodiments, R² is —OSO₃H, —OH, or —H. In some embodiments, R² is —OSO₃H. In some embodiments, R² is —SO₃H. In some embodiments, R² is —H. In some embodiments, R² is —OH.

In some embodiments, R⁴ is —SO₃H or —OSO₃H. In some embodiments, R⁴ is —SO₃H or —H. In some embodiments, R⁴ is —OSO₃H, —OH, or —H. In some embodiments, R⁴ is —OSO₃H. In some embodiments, R⁴ is —SO₃H. In some embodiments, R⁴ is —H. In some embodiments, R⁴ is —OH.

In some embodiments, R¹¹ is —SO₃H or —OSO₃H. In some embodiments, R¹¹ is —SO₃H or —H. In some embodiments, R¹¹ is —OSO₃H, —OH, or —H. In some embodiments, R¹¹ is —OSO₃H. In some embodiments, R¹¹ is —SO₃H. In some embodiments, R¹¹ is —H. In some embodiments, R¹¹ is —OH.

In some embodiments, R¹² is —SO₃H or —OSO₃H. In some embodiments, R¹² is —SO₃H or —H. In some embodiments, R¹² is —OSO₃H, —OH, or —H. In some embodiments, R² is —OSO₃H. In some embodiments, R¹² is —SO₃H. In some embodiments, R¹² is —H. In some embodiments, R² is —OH.

As defined herein, each of R³ and R¹⁰ is independently —SO₃H, —H, optionally substituted C₁-C₆ alkyl, or an oxygen protecting group. In some embodiments, each of R³ and R¹⁰ is independently —SO₃H or —H. In some embodiments, each of R³ and R¹⁰ is independently —H, optionally substituted C₁-C₆ alkyl, or an oxygen protecting group.

In some embodiments, R³ is —SO₃H, —H, optionally substituted C₁-C₆ alkyl, or an oxygen protecting group. In some embodiments, R³ is —SO₃H or —H. In some embodiments, R³ is —SO₃H. In some embodiments, R³ is —H. In some embodiments, R³ is —H, optionally substituted C₁-C₆ alkyl, or an oxygen protecting group. In some embodiments, R³ is —H or optionally substituted C₁-C₆ alkyl. In some embodiments, R³ is —H or an oxygen protecting group. In some embodiments, R³ is optionally substituted C₁-C₆ alkyl. In some embodiments, R³ is unsubstituted C₁-C₆ alkyl. In some embodiments, R³ is optionally substituted C₁-C₄ alkyl. In some embodiments, R³ is unsubstituted C₁-C₄ alkyl. In some embodiments, R³ is methyl. In some embodiments, R³ is ethyl. In some embodiments, R³ is an oxygen protecting group. In some embodiments, R³ is a silyl, TBDPS, TBDMS, TIPS, TES, TMS, MOM, THP, t-Bu, Bn, allyl, acetyl, pivaloyl, or benzoyl oxygen protecting group.

In some embodiments, R¹⁰ is —SO₃H, —H, optionally substituted C₁-C₆ alkyl, or an oxygen protecting group. In some embodiments, R¹⁰ is —SO₃H or —H. In some embodiments, R¹⁰ is —SO₃H. In some embodiments, R¹⁰ is —H. In some embodiments, R¹⁰ is —H, optionally substituted C₁-C₆ alkyl, or an oxygen protecting group. In some embodiments, R¹⁰ is —H or optionally substituted C₁-C₆ alkyl. In some embodiments, R¹⁰ is —H or an oxygen protecting group. In some embodiments, R¹⁰ is optionally substituted C₁-C₆ alkyl. In some embodiments, R¹⁰ is unsubstituted C₁-C₆ alkyl. In some embodiments, R¹⁰ is optionally substituted C₁-C₄ alkyl. In some embodiments, R¹⁰ is unsubstituted C₁-C₄ alkyl. In some embodiments, R¹⁰ is methyl. In some embodiments, R¹⁰ is ethyl. In some embodiments, R¹⁰ is an oxygen protecting group. In some embodiments, R³ is a silyl, TBDPS, TBDMS, TIPS, TES, TMS, MOM, THP, t-Bu, Bn, allyl, acetyl, pivaloyl, or benzoyl oxygen protecting group.

As defined herein, R is —OR^{B}, —SR^{B}, —N(R^{B})₂, halogen, an optionally substituted monosaccharide, or an optionally substituted oligosaccharide. In some embodiments, R⁵ is —OR^{B}, —SR^{B}, —N(R^{B})₂, halogen, or an optionally substituted monosaccharide. In some embodiments, R¹ is —OR^{B}, —SR^{B}, —N(R^{B})₂, or halogen. In some embodiments, R⁵ is an optionally substituted monosaccharide or an optionally substituted oligosaccharide. In some embodiments, R⁵ is —OR^{B}, —SR^{B}, or —N(R^{B})₂. In some embodiments, R⁵ is —OR^{B} or —SR^{B}. In some embodiments, R⁵ is —OR^{B} or —N(R^{B})₂. In some embodiments, R⁵ is —SR^{B} or —N(R^{B})₂. In some embodiments, R⁵ is —OR^{B}. In some embodiments, R⁵ is —OH or —O(oxygen protecting group). In some embodiments, R⁵ is —SR^{A}. In some embodiments, R⁵ is —SH or —S(sulfur protecting group). In some embodiments, R⁵ is —N(R^{B})₂. In some embodiments, R⁵ is —NH₂ or —NH(nitrogen protecting group). In some embodiments, R is halogen. In some embodiments, R is —F, —Cl, —Br, or —I. In some embodiments, R⁵ is —F, —Cl, or —Br. In some embodiments, R⁵ is —F or —Cl. In some embodiments, R⁵ is —Cl or —Br. In some embodiments, R⁵ is —F. In some embodiments, R⁵ is —Cl. In some embodiments, R⁵ is —Br. In some embodiments, R is —I. In some embodiments, R⁵ is an optionally substituted monosaccharide. In some embodiments, R⁵ is an optionally substituted oligosaccharide. In some embodiments, R⁵ is an optionally substituted glucuronide.

In some embodiments, R⁵ is

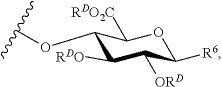

wherein: R^{D} is —H, optionally substituted C₁-C₆ alkyl, or an oxygen protecting group; R⁶ is —OR^{E}, —SR^{E}, or —N(R^{E})₂; and each occurrence of R^{E} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{E} are joined together with their intervening atoms to form an optionally substituted heterocyclic ring or optionally substituted heteroaryl ring.

As defined herein, R^{D} is —H, optionally substituted C₁-C₆ alkyl, or an oxygen protecting group. In some embodiments, R^{D} is —H or optionally substituted C₁-C₆ alkyl. In some embodiments, R^{D} is —H or an oxygen protecting group. In some embodiments, R^{D} is —H. In some embodiments, R^{D} is optionally substituted C₁-C₆ alkyl. In some embodiments, R^{D} is optionally substituted C₁-C₄ alkyl. In some embodiments, R^{D} is methyl. In some embodiments, R^{D} is ethyl. In some embodiments, R^{D} is an oxygen protecting group. In some embodiments, R^{D} is a silyl, TBDPS, TBDMS, TIPS, TES, TMS, MOM, THP, t-Bu, Bn, allyl, acetyl, pivaloyl, or benzoyl oxygen protecting group.

As defined herein, each occurrence of R^{E} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{E} are joined together with their intervening atoms to form an optionally substituted heterocyclic ring or optionally substituted heteroaryl ring. In some embodiments, each instance of R^{E} is independently —H, optionally substituted alkyl, optionally substituted alkenyl, optionally substituted alkynyl, optionally substituted heteroalkyl, optionally substituted heteroalkenyl, or optionally substituted heteroalkynyl. In some embodiments, each instance of R^{E} is optionally substituted carbocyclyl, optionally substituted heterocyclyl, optionally substituted aryl, or optionally substituted heteroaryl. In some embodiments, each instance of R^{E} is independently —H, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, each instance of R^{E} is independently —H, optionally substituted C₁₋₁₀ alkyl, or optionally substituted phenyl. In some embodiments, R^{E} is —H, a nitrogen protecting group when attached to a nitrogen atom, an oxygen protecting group when attached to an oxygen atom, or a sulfur protecting group when attached to a sulfur atom. In some embodiments, R^{E} is —H. In some embodiments, each instance of R^{E} is a nitrogen protecting group when attached to a nitrogen atom, an oxygen protecting group when attached to an oxygen atom, or a sulfur protecting group when attached to a sulfur atom. In some embodiments, two instances of R^{E} attached to the same intervening atom are joined together with the intervening atom to form an optionally substituted, monocyclic, heterocyclic or heteroaryl ring.

As defined herein, R⁶ is —OR^{E}, —SR^{E}, or —N(R^{E})₂. In some embodiments, R⁶ is —OR^{E} or —SR^{E}. In some embodiments, R⁶ is —OR^{E} or —N(R^{E})₂. In some embodiments, R⁶ is —SR^{E}, or —N(R^{E})₂. In some embodiments, R⁶ is —OR^{E}. In some embodiments, R⁶ is —OH or —O(oxygen protecting group). In some embodiments, R⁶ is —O(optionally substituted phenyl). In some embodiments, R⁶ is —SR^{E}. In some embodiments, R⁶ is —SH or —S(sulfur protecting group). In some embodiments, R⁶ is —S(optionally substituted phenyl). In some embodiments, R⁶ is —N(R^{E})₂. In some embodiments, R⁶ is —NH₂ or —NH(nitrogen protecting group). In some embodiments, R⁶ is —N(R^{E})(nitrogen protecting group).

In some embodiments, R⁶ is

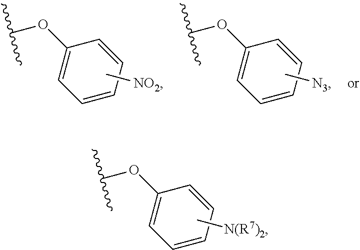

wherein: each occurrence of R⁷ is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or two occurrences of R⁷ are joined together with their intervening atoms to form an optionally substituted heterocyclic ring or optionally substituted heteroaryl ring.

In some embodiments, R⁶ is

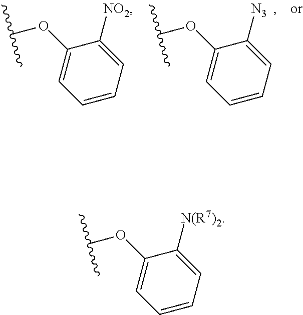

In some embodiments, R⁶ is

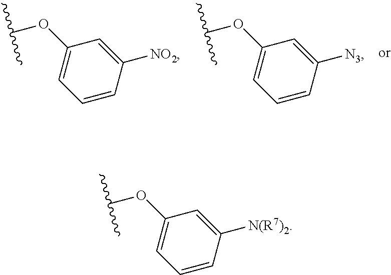

In some embodiments, R⁶ is

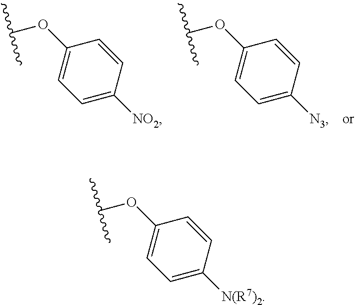

In some embodiments, R⁶ is

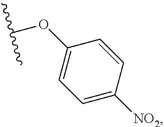 [SMILES: *Oc1ccc([N+](=O)[O-])cc1]

In some embodiments, R⁶ is

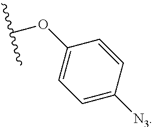 [SMILES: *Oc1ccc(N=[N+]=[N-])cc1]

In some embodiments, R⁶ is

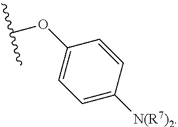

As defined herein, each occurrence of R⁷ is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or two occurrences of R⁷ are joined together with their intervening atoms to form an optionally substituted heterocyclic ring or optionally substituted heteroaryl ring. In some embodiments, each instance of R⁷ is independently —H, optionally substituted alkyl, optionally substituted alkenyl, optionally substituted alkynyl, optionally substituted heteroalkyl, optionally substituted heteroalkenyl, or optionally substituted heteroalkynyl. In some embodiments, each instance of R⁷ is optionally substituted carbocyclyl, optionally substituted heterocyclyl, optionally substituted aryl, or optionally substituted heteroaryl. In some embodiments, each instance of R⁷ is independently —H, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, each instance of R⁷ is independently —H, optionally substituted C₁₋₁₀ alkyl, or optionally substituted phenyl. In some embodiments, R⁷ is —H, or a nitrogen protecting group. In some embodiments, R⁷ is —H. In some embodiments, R⁷ is a nitrogen protecting group. In some embodiments, two instances of R⁷ attached to the same intervening atom are joined together with the intervening atom to form an optionally substituted, monocyclic, heterocyclic or heteroaryl ring.

As defined herein, each occurrence of R^{B} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{B} are joined together with their intervening atoms to form an optionally substituted heterocyclic ring or optionally substituted heteroaryl ring. In some embodiments, each instance of R^{B} is independently —H, optionally substituted alkyl, optionally substituted alkenyl, optionally substituted alkynyl, optionally substituted heteroalkyl, optionally substituted heteroalkenyl, or optionally substituted heteroalkynyl. In some embodiments, each instance of R^{B} is optionally substituted carbocyclyl, optionally substituted heterocyclyl, optionally substituted aryl, or optionally substituted heteroaryl. In some embodiments, each instance of R^{B} is independently —H, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, each instance of R^{B} is independently —H, optionally substituted C₁₋₁₀ alkyl, or optionally substituted phenyl. In some embodiments, R^{B} is —H, a nitrogen protecting group when attached to a nitrogen atom, an oxygen protecting group when attached to an oxygen atom, or a sulfur protecting group when attached to a sulfur atom. In some embodiments, R^{B} is —H. In some embodiments, each instance of R^{B} is a nitrogen protecting group when attached to a nitrogen atom, an oxygen protecting group when attached to an oxygen atom, or a sulfur protecting group when attached to a sulfur atom. In some embodiments, two instances of R^{B} attached to the same intervening atom are joined together with the intervening atom to form an optionally substituted, monocyclic, heterocyclic or heteroaryl ring.

As defined herein, each occurrence of R^{C} is independently —H, optionally substituted C₁-C₆ alkyl, or an oxygen protecting group. In some embodiments, each occurrence of R^{C} is independently —H or optionally substituted C₁-C₆ alkyl. In some embodiments, each occurrence of R^{C} is independently —H or an oxygen protecting group. In some embodiments, R^{C} is —H. In some embodiments, R^{C} is optionally substituted C₁-C₆ alkyl. In some embodiments, R^{C} is unsubstituted C₁-C₆ alkyl. In some embodiments, R^{C} is optionally substituted C₁-C₄ alkyl. In some embodiments, R^{C} is unsubstituted C₁-C₄ alkyl. In some embodiments, R^{C} is methyl. In some embodiments, R^{C} is ethyl. In some embodiments, R^{C} is an oxygen protecting group. In some embodiments, R^{C} is a silyl, TBDPS, TBDMS, TIPS, TES, TMS, MOM, THP, t-Bu, Bn, allyl, acetyl, pivaloyl, or benzoyl oxygen protecting group.

In some embodiments, the prodrug is an ester prodrug, a PEG ester prodrug, a Schiff base prodrug, an acetal prodrug, or a hemi-acetal prodrug. In some embodiments, the prodrug includes a linkage that can be enzymatically or hydrolytically cleaved under in vivo conditions.

In some embodiments, the heparin oligomer, or a pharmaceutically acceptable salt, solvate, hydrate, polymorph, co-crystal, tautomer, stereoisomer, isotopically labeled derivative, or prodrug thereof, comprises:

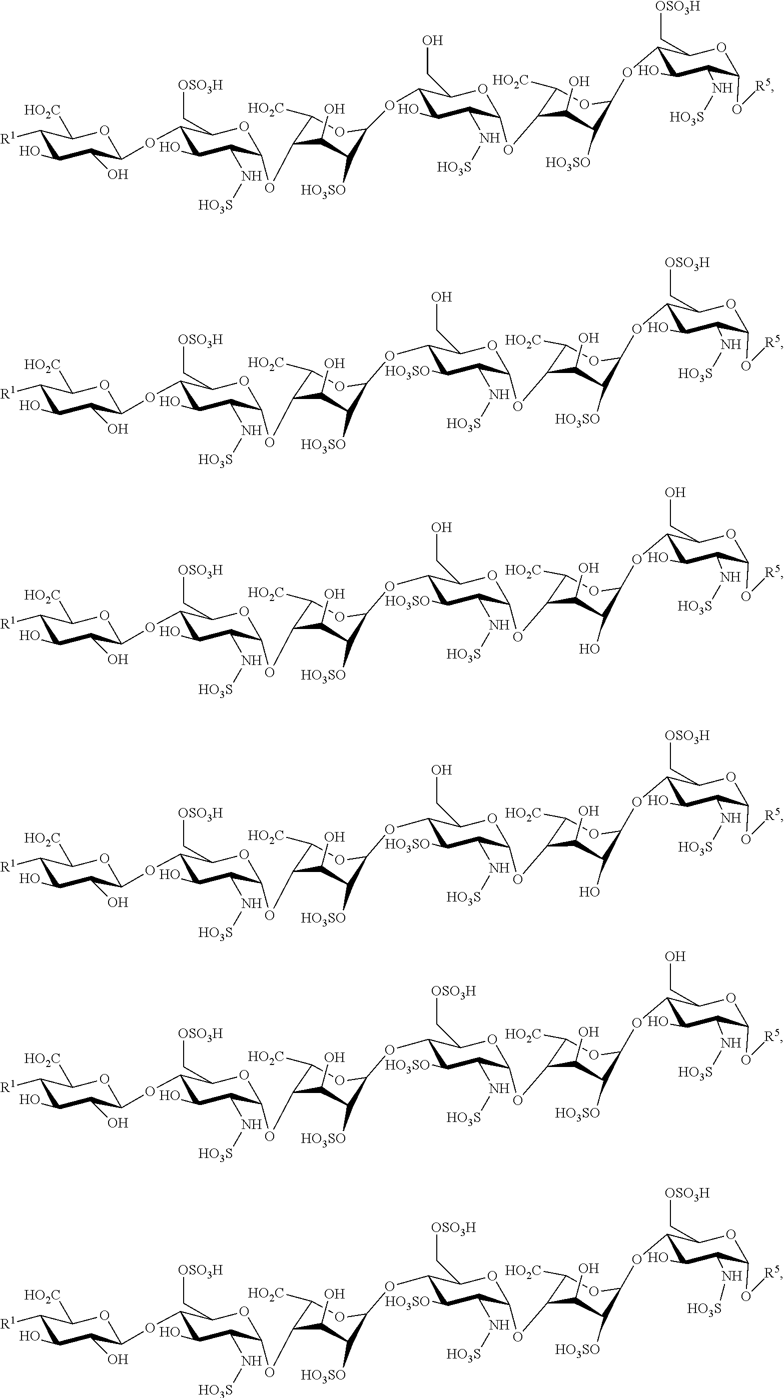

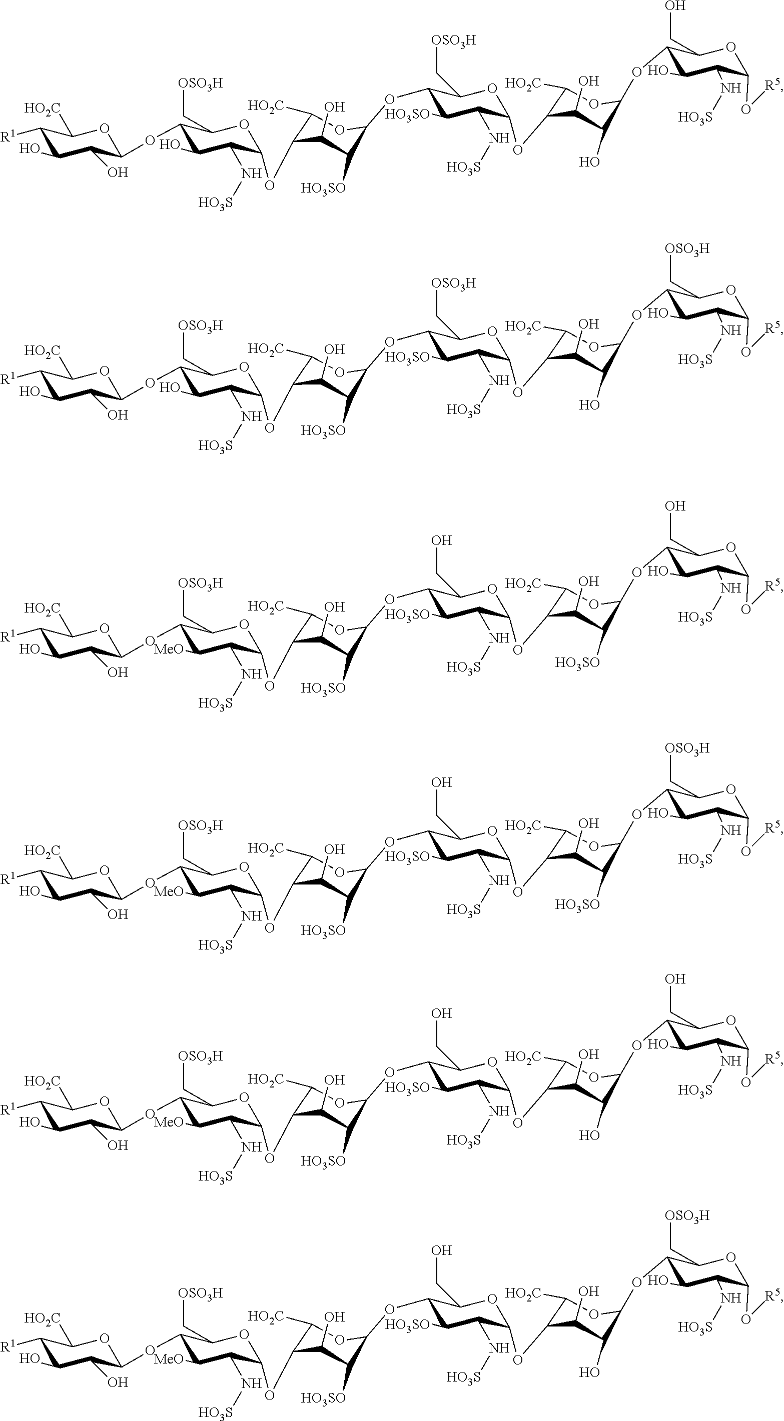

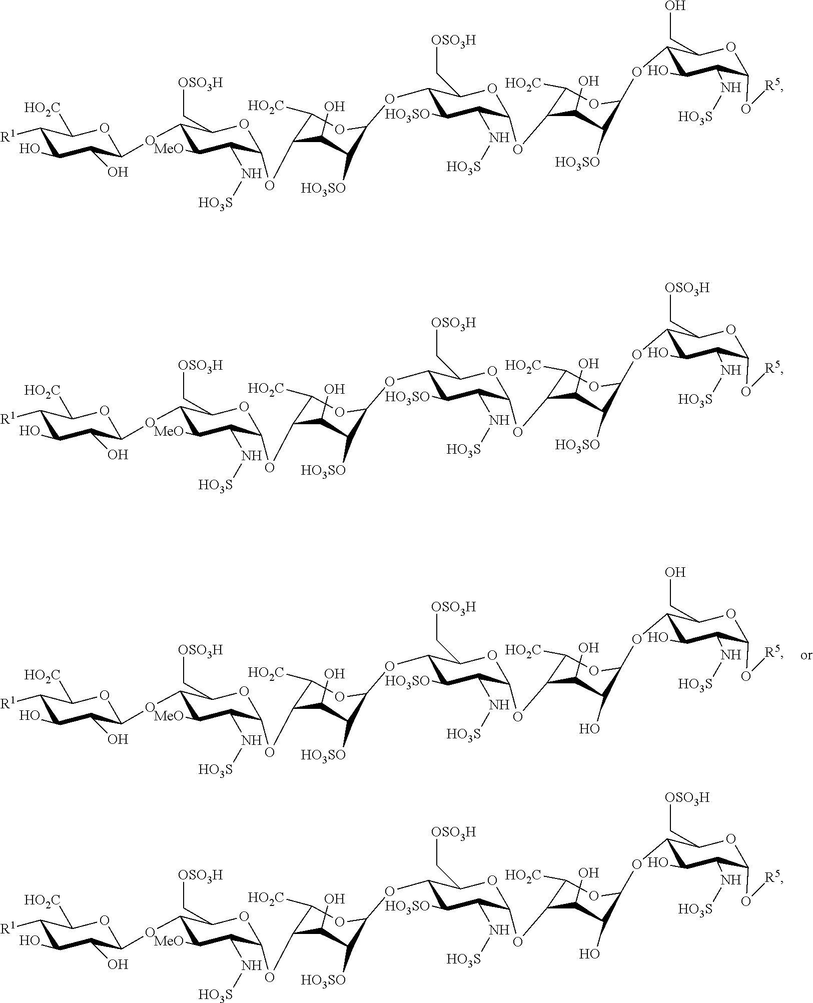

or a pharmaceutically acceptable salt, solvate, hydrate, polymorph, co-crystal, tautomer, stereoisomer, isotopically labeled derivative, or prodrug thereof.

In some embodiments, the heparin oligomer, or a pharmaceutically acceptable salt, solvate, hydrate, polymorph, co-crystal, tautomer, stereoisomer, isotopically labeled derivative, or prodrug thereof, comprises:

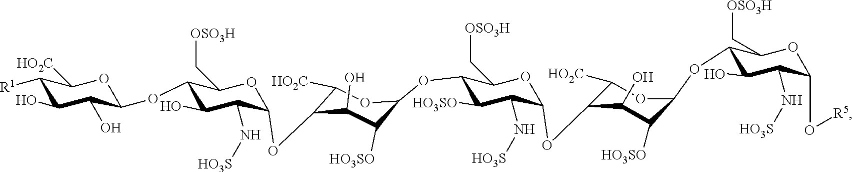

or a pharmaceutically acceptable salt, solvate, hydrate, polymorph, co-crystal, tautomer, stereoisomer, isotopically labeled derivative, or prodrug thereof.

In some embodiments, the heparin oligomer is a heparin heptamer. In some embodiments, the heparin heptamer has the structure:

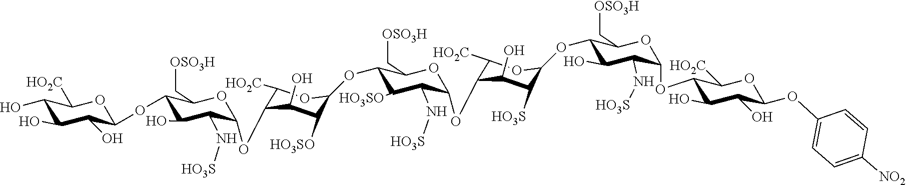

or a pharmaceutically acceptable salt, solvate, hydrate, polymorph, co-crystal, tautomer, stereoisomer, isotopically labeled derivative, or prodrug thereof.

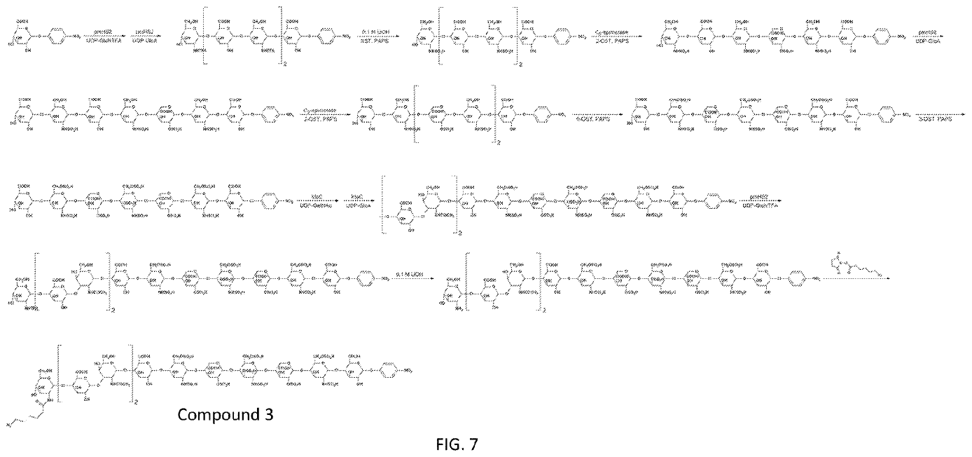

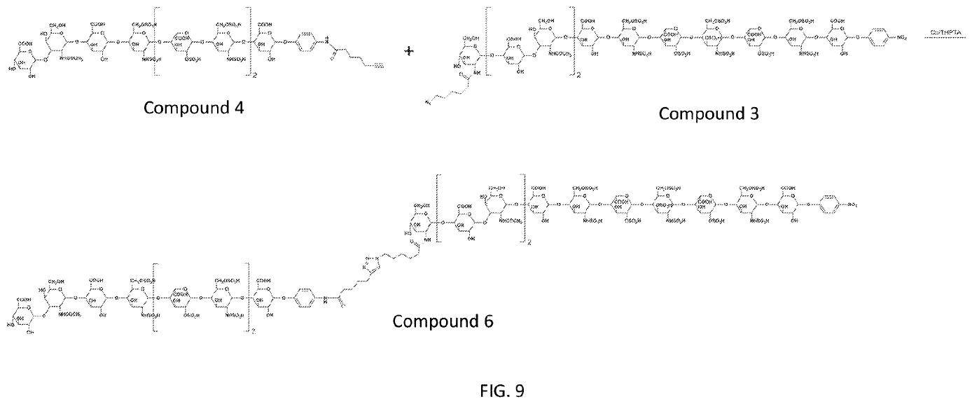

In some embodiments, the heparin oligomer is Compound 3, whose structure is shown in FIG. 7, or a pharmaceutically acceptable salt, solvate, hydrate, polymorph, co-crystal, tautomer, stereoisomer, isotopically labeled derivative or prodrug thereof. In some embodiments, the heparin oligomer is Compound 6, whose structure is shown in FIG. 9, or a pharmaceutically acceptable salt, solvate, hydrate, polymorph, co-crystal, tautomer, stereoisomer, isotopically labeled derivative or prodrug thereof.

In some embodiments, the heparin oligomer is resistant to heparinase degradation. In some embodiments, the

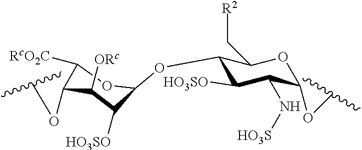

linkage is resistant to heparinase degradation. In some embodiments, the

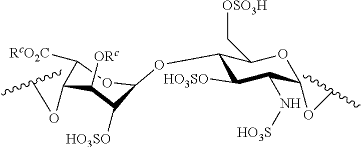

linkage is resistant to heparinase degradation. In some embodiments, the

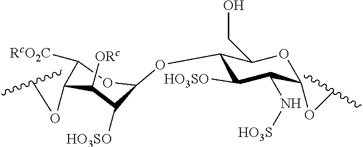

linkage is resistant to heparinase degradation. In some embodiments, the

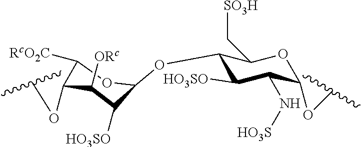

linkage is resistant to heparinase degradation.
In some embodiments, the

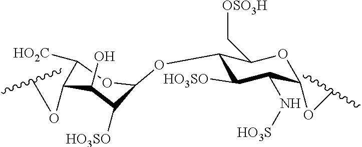

(i.e., -[IdoA2S-GlcNS3S6S]—) linkage is resistant to heparinase degradation. In some embodiments, the

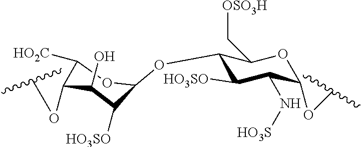

(i.e., -[IdoA2S-GlcNS3S]—) linkage is resistant to heparinase degradation. In some embodiments, the

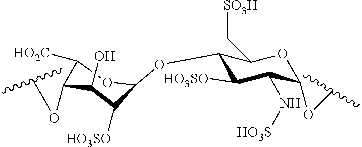

linkage is resistant to heparinase degradation. In some embodiments, the heparin oligomer has anti-FXa and/or anti-FIIa activity. In some embodiments, the heparin oligomer has anti-FXa activity. In some embodiments, the heparin oligomer has anti-FIIa activity. In some embodiments, the heparin oligomer has anti-FXa activity and anti-FIIa activity.

### Polymer Conjugates, Oligosaccharide Conjugates, and Oligomeric Compounds

In another aspect, the present disclosure provides oligomeric compounds comprising two or more repeat units connected via a linker, and pharmaceutically acceptable salts, solvates, hydrates, polymorphs, co-crystals, tautomers, stereoisomers, isotopically labeled derivatives, and prodrugs thereof, wherein the repeat units are each independently a heparin oligomer provided herein. In some embodiments, the repeat units are each independently a heparin heptamer provided herein.

In some embodiments, the oligomeric compound has a linear structure.

In some embodiments, the oligomeric compound comprises three or more heparin oligomers. In some embodiments, the oligomeric compound comprises four or more heparin oligomers. In some embodiments, the oligomeric compound comprises five or more heparin oligomers. In some embodiments, the oligomeric compound comprises ten or more heparin oligomers.

In another aspect, the present disclosure provides polymer conjugates, and pharmaceutically acceptable salts, solvates, hydrates, polymorphs, co-crystals, tautomers, stereoisomers, isotopically labeled derivatives, and prodrugs thereof, wherein the polymer conjugate comprises a heparin oligomer or oligomeric compound provided herein, or a pharmaceutically acceptable salt, solvate, hydrate, polymorph, co-crystal, tautomer, stereoisomer, isotopically labeled derivative, or prodrug thereof, conjugated to a polymer via a linker. In some embodiments, the polymer conjugate comprises a heparin heptamer provided herein.

In some embodiments, the linker is a bond, —C═O—, —O—, —S—, —NR^{F}—, —NR^{F}C(═O)—, —C(═O)NR^{F}—, —SC(═O)—, —C(═O)S—, —OC(═O)—, —C(═O)O—, —NR^{F}C(═S)—, —C(═S)NR^{F}—, —S(═O)—, —S(═O)O—, —OS(═O)—, —S(═O)NR^{F}—, —NR^{F}S(═O)—, —S(═O)₂—, —S(═O)₂O—, —OS(═O)₂—, —S(═O)₂NR^{F}—, —NR^{F}S(═O)₂—, an optionally substituted monosaccharide, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or any combination thereof, wherein R^{F} is hydrogen, substituted or unsubstituted acyl, substituted or unsubstituted, C₁₋₆ alkyl, or a nitrogen protecting group. In some embodiments, the linker is a bond, an optionally substituted monosaccharide, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or any combination thereof. In some embodiments, the linker is a bond, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or any combination thereof. In some embodiments, the linker is a bond. In some embodiments, the linker is an optionally substituted monosaccharide. In some embodiments, the linker is optionally substituted alkylene, optionally substituted alkenylene, optionally substituted alkynylene, optionally substituted heteroalkylene, optionally substituted heteroalkenylene, or optionally substituted heteroalkynylene. In some embodiments, the linker is optionally substituted carbocyclylene, optionally substituted heterocyclylene, optionally substituted arylene, or optionally substituted heteroarylene. In some embodiments, the linker is a bond, —NHC(═O)—, or an optionally substituted saccharide. In some embodiments, the linker is a bond or —NHC(═O)—. In some embodiments, the linker is a bond or an optionally substituted saccharide. In some embodiments, the linker is —NHC(═O)— or an optionally substituted saccharide. In some embodiments, the linker is —NHC(═O)—.

In some embodiments, the linker comprises optionally substituted C₁-C₂₀ alkylene, optionally substituted C₂-C₂₀ alkenylene, optionally substituted C₂-C₂₀ alkynylene. In some embodiments, the linker comprises optionally substituted C₁-C₁₀ alkylene, optionally substituted C₂-C₂₀ alkenylene, optionally substituted C₂-C₁₀ alkynylene. In some embodiments, the linker comprises optionally substituted C₁-C₆ alkylene, optionally substituted C₂-C₆ alkenylene, optionally substituted C₂-C₆ alkynylene.

In some embodiments, the linker comprises optionally substituted C₁-C₂₀ heteroalkylene, optionally substituted C₁-C₂₀ heteroalkenylene, or optionally substituted C₁-C₂₀ heteroalkynylene. In some embodiments, the linker comprises optionally substituted C₁-C₁₀ heteroalkylene, optionally substituted C₂-C₁₀ heteroalkenylene, or optionally substituted C₂-C₁₀ heteroalkynylene. In some embodiments, the linker comprises optionally substituted C₁-C₆ heteroalkylene, optionally substituted C₂-C₆ heteroalkenylene, or optionally substituted C₂-C₆ heteroalkynylene.

In some embodiments, the linker comprises optionally substituted carbocyclylene or optionally substituted heterocyclylene. In some embodiments, the linker comprises optionally substituted C₃-C₁₄ carbocyclylene. In some embodiments, the linker comprises optionally substituted C₃-C₇ carbocyclylene. In some embodiments, the linker comprises optionally substituted 3- to 14-membered heterocyclylene. In some embodiments, the linker comprises optionally substituted 3- to 7-membered heterocyclylene.

In some embodiments, the linker comprises optionally substituted arylene, or optionally substituted heteroarylene. In some embodiments, the linker comprises optionally substituted C₆-C₁₄ arylene. In some embodiments, the linker comprises optionally substituted C₆-C₁₀ arylene. In some embodiments, the linker comprises optionally substituted 5- to 14-membered heteroarylene. In some embodiments, the linker comprises optionally substituted 5- to 10-membered heteroarylene.

As defined herein, R^{F} is hydrogen, substituted or unsubstituted acyl, substituted or unsubstituted, C₁₋₆ alkyl, or a nitrogen protecting group. In some embodiments, R^{F} is hydrogen. In some embodiments, R^{F} is substituted or unsubstituted acyl. In some embodiments, R^{F} is substituted or unsubstituted C₁₋₆ alkyl. In some embodiments, R^{F} is a nitrogen protecting group

The polymer may be conjugated via the linker to any part of the heparin oligomer or oligomeric compound. For example, the polymer may be conjugated via the linker to a moiety such as a hydroxy, amino, —COOH, —OSO₃H, or —NHSO₃H. In some embodiments, the polymer is conjugated to an interior (non-terminal) saccharide of the heparin oligomer. In some embodiments, the polymer is conjugated to either terminus of the heparin oligomer. In some embodiments, R⁶ of the heparin oligomer is —O(optionally substituted phenyl) substituted with the linker.

In some embodiments, the polymer is a polyethylene glycol, a polyacrylate, a polyester, a polycarbonate, a polyolefin, a polyamide, or any combination thereof. In some embodiments, the polymer is a polyethylene glycol, a polyester, a polycarbonate, or a polyamide, or any combination thereof. In some embodiments, the polymer is a polyester, a polycarbonate, or a polyamide, or any combination thereof. In some embodiments, the polymer is a polyethylene glycol, a polyester, or a polycarbonate, or any combination thereof. In some embodiments, the polymer is a polyacrylate, a polyolefin, or any combination thereof. In some embodiments, the polymer is a polyethylene glycol. In some embodiments, the polymer is a polyacrylate. In some embodiments, the polymer is a polyester. In some embodiments, the polymer is a polycarbonate. In some embodiments, the polymer is a polyolefin. In some embodiments, the polymer is a polyamide.

In some embodiments, the polymer has a molecular weight of about 1,000 Da to about 1,000,000 Da. In some embodiments, the polymer has a molecular weight of about 1,000 Da to about 500,000 Da. In some embodiments, the polymer has a molecular weight of about 1,000 Da to about 200,000 Da. In some embodiments, the polymer has a molecular weight of about 1,000 Da to about 100,000 Da. In some embodiments, the polymer has a molecular weight of about 1,000 Da to about 50,000 Da. In some embodiments, the polymer has a molecular weight of about 1,000 Da to about 20,000 Da. In some embodiments, the polymer has a molecular weight of about 1,000 Da to about 10,000 Da. In some embodiments, the polymer has a molecular weight of about 1,000 Da to about 5,000 Da. In some embodiments, the polymer has a molecular weight of about 2,000 Da to about 1,000,000 Da. In some embodiments, the polymer has a molecular weight of about 2,000 Da to about 500,000 Da. In some embodiments, the polymer has a molecular weight of about 2,000 Da to about 200,000 Da. In some embodiments, the polymer has a molecular weight of about 2,000 Da to about 100,000 Da. In some embodiments, the polymer has a molecular weight of about 2,000 Da to about 50,000 Da. In some embodiments, the polymer has a molecular weight of about 2,000 Da to about 20,000 Da. In some embodiments, the polymer has a molecular weight of about 2,000 Da to about 10,000 Da. In some embodiments, the polymer has a molecular weight of about 5,000 Da to about 1,000,000 Da. In some embodiments, the polymer has a molecular weight of about 5,000 Da to about 500,000 Da. In some embodiments, the polymer has a molecular weight of about 5,000 Da to about 200,000 Da. In some embodiments, the polymer has a molecular weight of about 5,000 Da to about 100,000 Da. In some embodiments, the polymer has a molecular weight of about 5,000 Da to about 50,000 Da. In some embodiments, the polymer has a molecular weight of about 5,000 Da to about 20,000 Da. In some embodiments, the polymer has a molecular weight of about 5,000 Da to about 10,000 Da.

In some embodiments, molecular weight is Mₙ. In some embodiments, molecular weight is M_{w}.

In some embodiments, the polymer has a Mₙ of about 1,000 Da to about 1,000,000 Da. In some embodiments, the polymer has a Mₙ of about 1,000 Da to about 500,000 Da. In some embodiments, the polymer has a Mₙ of about 1,000 Da to about 200,000 Da. In some embodiments, the polymer has a Mₙ of about 1,000 Da to about 100,000 Da. In some embodiments, the polymer has a Mₙ of about 1,000 Da to about 50,000 Da. In some embodiments, the polymer has a Mₙ of about 1,000 Da to about 20,000 Da. In some embodiments, the polymer has a Mₙ of about 1,000 Da to about 10,000 Da. In some embodiments, the polymer has a Mₙ of about 1,000 Da to about 5,000 Da. In some embodiments, the polymer has a Mₙ of about 2,000 Da to about 1,000,000 Da. In some embodiments, the polymer has a Mₙ of about 2,000 Da to about 500,000 Da. In some embodiments, the polymer has a Mₙ of about 2,000 Da to about 200,000 Da. In some embodiments, the polymer has a Mₙ of about 2,000 Da to about 100,000 Da. In some embodiments, the polymer has a Mₙ of about 2,000 Da to about 50,000 Da. In some embodiments, the polymer has a Mₙ of about 2,000 Da to about 20,000 Da. In some embodiments, the polymer has a Mₙ of about 2,000 Da to about 10,000 Da. In some embodiments, the polymer has a M. of about 5,000 Da to about 1,000,000 Da. In some embodiments, the polymer has a Mₙ of about 5,000 Da to about 500,000 Da. In some embodiments, the polymer has a Mₙ of about 5,000 Da to about 200,000 Da. In some embodiments, the polymer has a Mₙ of about 5,000 Da to about 100,000 Da. In some embodiments, the polymer has a Mₙ of about 5,000 Da to about 50,000 Da. In some embodiments, the polymer has a Mₙ of about 5,000 Da to about 20,000 Da. In some embodiments, the polymer has a Mₙ of about 5,000 Da to about 10,000 Da.

In some embodiments, the polymer comprises one or more additional instances of a heparin oligomer or oligomeric compound provided herein. In some embodiments, the polymer comprises two or more additional instances of a heparin oligomer or oligomeric compound provided herein. In some embodiments, the polymer comprises five or more additional instances of a heparin oligomer or oligomeric compound provided herein. In some embodiments, the polymer comprises ten or more additional instances of a heparin oligomer or oligomeric compound provided herein. In some embodiments, the polymer comprises fifteen or more additional instances of a heparin oligomer or oligomeric compound provided herein. In some embodiments, the polymer comprises twenty or more additional instances of a heparin oligomer or oligomeric compound provided herein. In some embodiments, the polymer comprises one to five additional instances of a heparin oligomer or oligomeric compound provided herein. In some embodiments, the polymer comprises one to ten additional instances of a heparin oligomer or oligomeric compound provided herein. In some embodiments, the polymer comprises one to fifteen additional instances of a heparin oligomer or oligomeric compound provided herein. In some embodiments, the polymer comprises one to twenty additional instances of a heparin oligomer or oligomeric compound provided herein. In some embodiments, the polymer comprises one to fifty additional instances of a heparin oligomer or oligomeric compound provided herein.

In some embodiments, the polymer comprises one or more additional instances of a heparin heptamer provided herein. In some embodiments, the polymer comprises two or more additional instances of a heparin heptamer provided herein. In some embodiments, the polymer comprises five or more additional instances of a heparin heptamer provided herein. In some embodiments, the polymer comprises ten or more additional instances of a heparin heptamer provided herein. In some embodiments, the polymer comprises fifteen or more additional instances of a heparin heptamer provided herein. In some embodiments, the polymer comprises twenty or more additional instances of a heparin heptamer provided herein. In some embodiments, the polymer comprises one to five additional instances of a heparin heptamer provided herein. In some embodiments, the polymer comprises one to ten additional instances of a heparin heptamer provided herein. In some embodiments, the polymer comprises one to fifteen additional instances of a heparin heptamer provided herein. In some embodiments, the polymer comprises one to twenty additional instances of a heparin heptamer provided herein. In some embodiments, the polymer comprises one to fifty additional instances of a heparin heptamer provided herein.

In some embodiments, the polymer comprises one or more additional instances of the heparin oligomer grafted onto a polymer backbone.

In some embodiments, the polymer conjugate has the structure

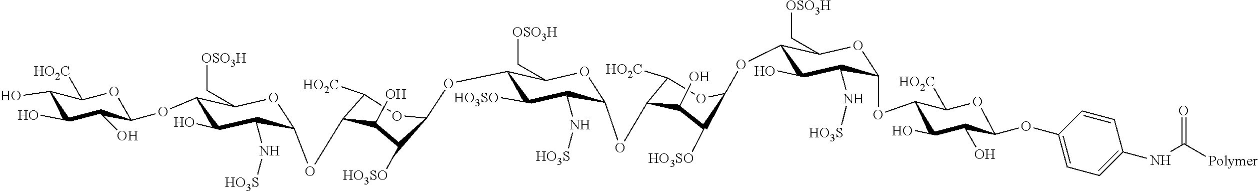

or a pharmaceutically acceptable salt, solvate, hydrate, polymorph, co-crystal, tautomer, stereoisomer, isotopically labeled derivative, or prodrug thereof.

In some embodiments, the polymer conjugate has the structure

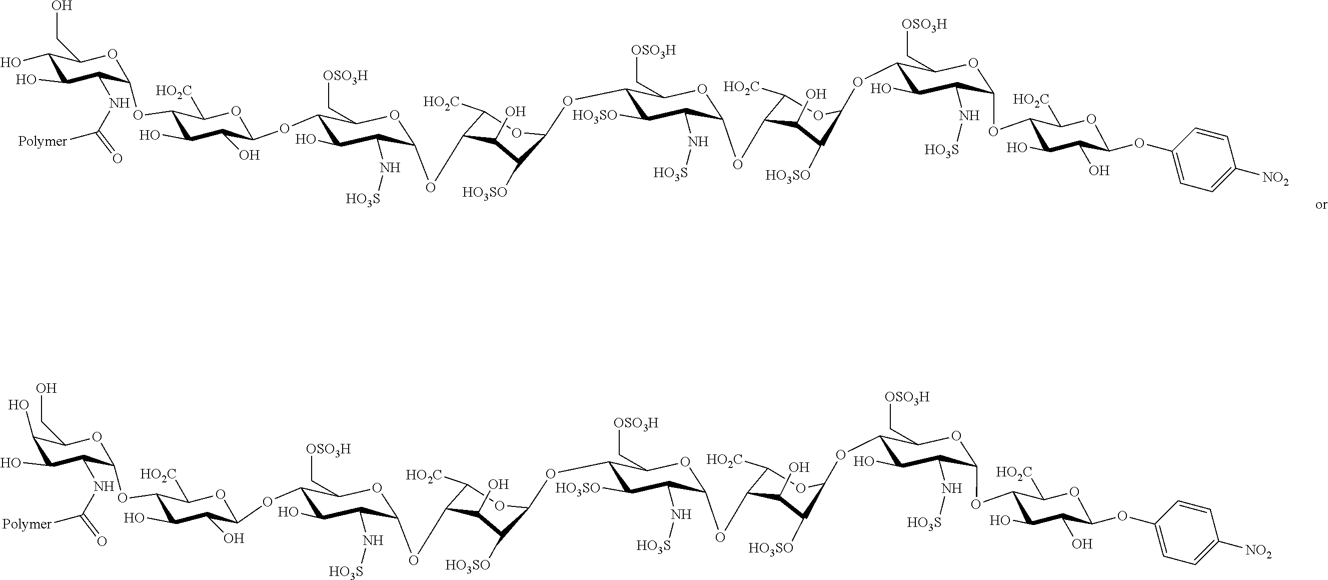

or a pharmaceutically acceptable salt, solvate, hydrate, polymorph, co-crystal, tautomer, stereoisomer, isotopically labeled derivative, or prodrug thereof.

In another aspect, the present disclosure provides conjugates of a heparin oligomer-containing domain and another oligosaccharide-containing domain, wherein the heparin oligomer-containing domain and the other oligosaccharide-containing domain are covalently attached to one another via a linker. The linker can be a carbohydrate linker, a non-carbohydrate linker, or a combination thereof. The conjugates can be prepared via any suitable conjugation chemistry, e.g., reactions such as those used to prepare conjugates of proteins or nucleic acids or using Click chemistry reactions, e.g., azide-alkyne cycloaddition reactions. However, any other

Thus, in some embodiments, the oligosaccharide conjugate comprises two domains: a first domain containing a monovalent derivative of a heparin oligomer as disclosed herein and a second domain containing a monovalent derivative of an optionally substituted oligosaccharide (e.g., an optionally substituted oligosaccharide that does not comprise a derivative of a heparin oligomer of Formula (I) as described herein). In some embodiments, the second domain comprises a moiety with anti-FIIa activity.

In some embodiments, the oligosaccharide conjugate has a structure of the formula:

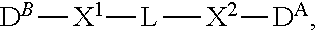

wherein: L is a bivalent linker; X¹ is present or absent and when present is an optionally substituted monosaccharide residue or an optionally substituted oligosaccharide residue; X² is present or absent and when present is an optionally substituted monosaccharide residue or an optionally substituted oligosaccharide residue; D^{A} is a heparin oligomer having a structure of Formula (I-B):

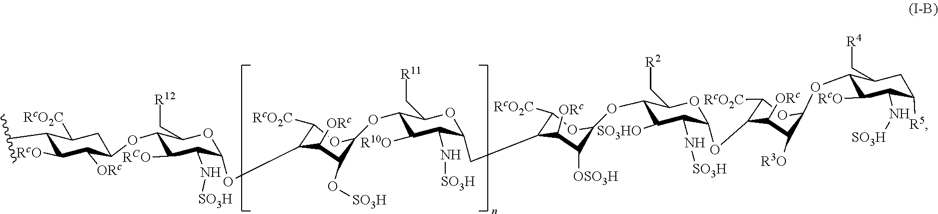

D^{B} is an oligosaccharide-containing oligomer having a structure of Formula (II-A):

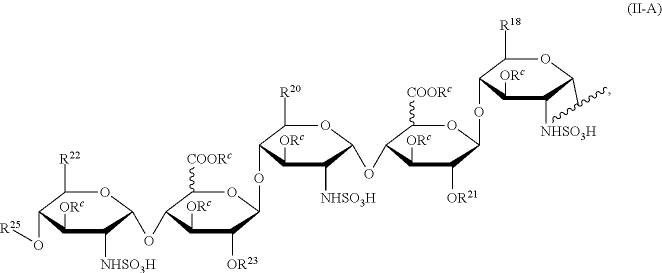

n is 1, 2, 3, 4, 5, 6, 7, 8, 8, 9, or 10; each of R², R⁴, R¹¹, R¹², R¹⁸, R²⁰, and R²² is independently —H, —OH, —OSO₃H, or —SO₃H; each of R³, R¹⁰, R²¹, and R²³ is independently —SO₃H, —H, optionally substituted C₁-C₆ alkyl, or an oxygen protecting group; R is —OR^{B}, —SR^{B}, —N(R^{B})₂, halogen, an optionally substituted monosaccharide, or an optionally substituted oligosaccharide; each occurrence of R^{B} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{B} are joined together with their intervening atoms to form an optionally substituted heterocyclic ring or optionally substituted heteroaryl ring; each occurrence of R^{C} is independently —H, optionally substituted C₁-C₆ alkyl, or an oxygen protecting group; and R²⁵ is —H, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aralkyl, optionally substituted aryl, optionally substituted heteroaryl, an oxygen protecting group, an optionally substituted monosaccharide, or an optionally substituted oligosaccharide.

In some embodiments, L is an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or any combination thereof. In some embodiments, L is: —O-phenyl-NH—C(═O)-(alkylene)-(triazolyl)-(alkylene)-C(═O)—. In some embodiments, each alkylene is independently a C₁-C₆ alkylene (e.g., methylene, ethylene, propylene, butylene, pentylene, or hexylene).

In some embodiments, X² is present. In some embodiments, X² comprises one or more optionally substituted galactosamine residues. In some embodiments, X² further comprises one or more optionally substituted glucuronic acid residues and/or one or more iduronic acid residues.

In some embodiments, R²⁵ is an optionally substituted oligosaccharide comprising one or more optionally substituted galactosamine residues. In some embodiments, R²⁵ further comprises one or more optionally substituted glucuronic acid residues and/or one or more iduronic acid residues.

In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, each of R², R⁴, R¹¹, and R¹² is independently —OH or —OSO₃H. In some embodiments, each of R², R⁴, R¹¹, and R² is —OH. In some embodiments, each of R², R⁴, R¹¹, and R¹² is —OSO₃H. In some embodiments, each of R¹¹, R²⁰, and R²² is independently —OH or —OSO₃H. In some embodiments, each of R¹⁸, R²⁰, and R²² is —OH. In some embodiments, each of R¹, R²⁰, and R²² is —OSO₃H. In some embodiments, each R^{C} is —H. In some embodiments, D^{A} comprises the structure:

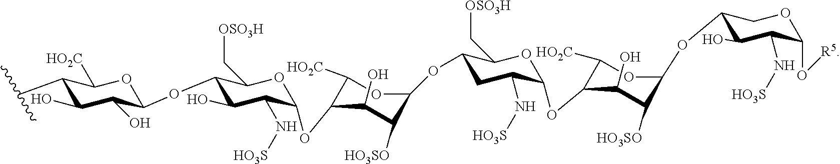

In some embodiments, D^{B} comprises the structure:

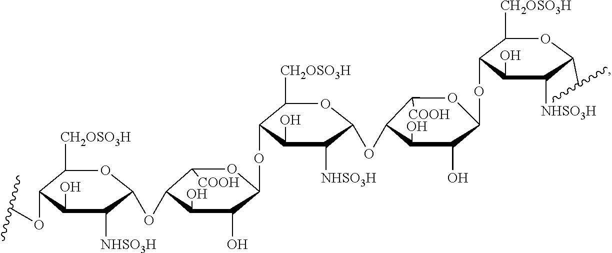

In some embodiments, the oligosaccharide conjugate is heparanase-resistant. In some embodiments, the oligosaccharide conjugate has anti-FXa and/or anti-FIIa activity. In some embodiments, the oligosaccharide conjugate has anti-FXa activity. In some embodiments, the oligosaccharide conjugate has anti-FIIa activity. In some embodiments, the oligosaccharide conjugate has anti-FXa and anti-FIIa activity.

In some embodiments, the oligosaccharide conjugate has the structure of Compound 6.

### Pharmaceutical Compositions, Kits, and Administration

In another aspect, the present disclosure provides pharmaceutical compositions comprising a heparin oligomer, oligomeric compound, polymer conjugate, or oligosaccharide conjugate provided herein, or a pharmaceutically acceptable salt, solvate, hydrate, polymorph, co-crystal, tautomer, stereoisomer, isotopically labeled derivative, or prodrug thereof, and a pharmaceutically acceptable excipient. In some embodiments, the pharmaceutical composition comprises a heparin heptamer provided herein, or a pharmaceutically acceptable salt, solvate, hydrate, polymorph, co-crystal, tautomer, stereoisomer, isotopically labeled derivative, or prodrug thereof, and a pharmaceutically acceptable excipient.

In certain embodiments, the compound described herein is provided in an effective amount in the pharmaceutical composition. In some embodiments, the effective amount is a therapeutically effective amount. In certain embodiments, a therapeutically effective amount is an amount sufficient for inhibiting thrombus formation. In certain embodiments, a therapeutically effective amount is an amount sufficient for treating a disease. In certain embodiments, a therapeutically effective amount is an amount sufficient for treating cardiovascular disease, atherosclerosis, atherosclerotic lesions, thrombus formation, thromboembolism, stroke, myocardial infarction, or primary or recurrent venous thromboembolism (VTE). In certain embodiments, a therapeutically effective amount is an amount sufficient for reducing thrombus formation and treating a disease. In certain embodiments, a therapeutically effective amount is an amount sufficient for inhibiting thrombus formation and treating a disease. In certain embodiments, a therapeutically effective amount is an amount sufficient for reducing thrombus formation and treating cardiovascular disease, atherosclerosis, atherosclerotic lesions, thrombus formation, thromboembolism, stroke, myocardial infarction, or primary or recurrent venous thromboembolism (VTE). In certain embodiments, a therapeutically effective amount is an amount sufficient for inhibiting thrombus formation and treating cardiovascular disease, atherosclerosis, atherosclerotic lesions, thrombus formation, thromboembolism, stroke, myocardial infarction, or primary or recurrent venous thromboembolism (VTE).

In some embodiments, the effective amount is a prophylactically effective amount. In certain embodiments, a prophylactically effective amount is an amount sufficient for reducing thrombus formation. In certain embodiments, a prophylactically effective amount is an amount sufficient for inhibiting thrombus formation. In certain embodiments, a prophylactically effective amount is an amount sufficient for preventing a disease. In certain embodiments, a prophylactically effective amount is an amount sufficient for preventing cardiovascular disease, atherosclerosis, atherosclerotic lesions, thrombus formation, thromboembolism, stroke, myocardial infarction, or primary or recurrent venous thromboembolism (VTE). In certain embodiments, a prophylactically effective amount is an amount sufficient for reducing thrombus formation and preventing a disease. In certain embodiments, a prophylactically effective amount is an amount sufficient for inhibiting thrombus formation and preventing a disease. In certain embodiments, a prophylactically effective amount is an amount sufficient for reducing thrombus formation and preventing cardiovascular disease, atherosclerosis, atherosclerotic lesions, thrombus formation, thromboembolism, stroke, myocardial infarction, or primary or recurrent venous thromboembolism (VTE). In certain embodiments, a prophylactically effective amount is an amount sufficient for inhibiting thrombus formation and preventing cardiovascular disease, atherosclerosis, atherosclerotic lesions, thrombus formation, thromboembolism, stroke, myocardial infarction, or primary or recurrent venous thromboembolism (VTE).

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the pharmaceutical composition is to be administered. The pharmaceutical composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the pharmaceutical composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span© 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol©), sucrose fatty acid esters, polyethylene glycol fatty acid esters (e.g., Cremophor*), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum©), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the pharmaceutical composition, and the active ingredient may constitute 0.1 to 20% (w/w) of the pharmaceutical composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface-active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the pharmaceutical compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the pharmaceutical compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

In some embodiments, the pharmaceutical composition is formulated for oral, intravenous, or subcutaneous administration. In some embodiments, the pharmaceutical composition is formulated for oral administration. In some embodiments, the pharmaceutical composition is formulated for oral administration and further comprises a carrier that complexes with a heparin oligomer provided herein. In some embodiments, the pharmaceutical composition is formulated for oral administration and further comprises a lipid. In some embodiments, the pharmaceutical composition is formulated for oral administration and further comprises deoxycholic acid. In some embodiments, the pharmaceutical composition is formulated for intravenous or subcutaneous administration. In some embodiments, the pharmaceutical composition is formulated for intravenous administration. In some embodiments, the pharmaceutical composition is formulated for subcutaneous administration.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

In some embodiments, a compound or composition, as described herein, is administered in combination with one or more additional pharmaceutical agents (e.g., therapeutically and/or prophylactically active agents). In some embodiments, the compounds or compositions are administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

In some embodiments, the compound or composition is administered concurrently with, prior to, or subsequent to one or more additional pharmaceutical agents. In some embodiments, the one or more additional agents are useful as, e.g., combination therapies. Pharmaceutical agents include therapeutically active agents. Pharmaceutical agents also include prophylactically active agents.

Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In some embodiments, the pharmaceutical composition further comprises an additional therapeutic agent.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is an binder or inhibitor of a protein kinase. In certain embodiments, the additional pharmaceutical agent is selected from the group comprising epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

Also encompassed by the disclosure are kits (e.g., pharmaceutical packs). The kits provided may comprise a pharmaceutical composition or compound described herein (e.g., a heparin oligomer, oligomeric compound, polymer conjugate, or oligosaccharide conjugate, or pharmaceutically acceptable salt, solvate, hydrate, co-crystal, tautomer, stereoisomer, isotopically labeled derivative, or prodrug thereof) and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease in a subject in need thereof. In certain embodiments, the kits are useful for preventing a disease in a subject in need thereof. In certain embodiments, the kits are useful for reducing the risk of developing a disease in a subject in need thereof. In certain embodiments, the kits are useful for inhibiting thrombus formation. In some embodiments, the kits are useful for reducing thrombus formation.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease in a subject in need thereof. In certain embodiments, the kits and instructions provide for preventing a disease in a subject in need thereof. In certain embodiments, the kits and instructions provide for reducing the risk of developing a disease in a subject in need thereof. In certain embodiments, the kits and instructions provide for inhibiting thrombus formation. In certain embodiments, the kits and instructions provide for reducing thrombus formation. A kit described herein may include one or more additional pharmaceutical agents described herein as a separate composition.

### Surface Coatings and Devices

In another aspect, the present disclosure provides surface coatings comprising a heparin oligomer, oligomeric compound, polymer conjugate, or oligosaccharide conjugate provided herein, or a pharmaceutically acceptable salt, solvate, hydrate, polymorph, co-crystal, tautomer, stereoisomer, isotopically labeled derivative, or prodrug thereof, and an excipient. In some embodiments, the surface coating comprises a heparin heptamer provided herein, or a pharmaceutically acceptable salt, solvate, hydrate, polymorph, co-crystal, tautomer, stereoisomer, isotopically labeled derivative, or prodrug thereof, and an excipient.

In some embodiments, the surface coating comprises one or more excipients. In certain embodiments, the surface coating comprises one or more of a solvent, a polymer, a fat and/or wax, a plasticizer, a colorant or any combination thereof. Polymers, plasticizers, colorants, solvents, fats, and/or waxes may be combined in any suitable amount to form the coating.

In some embodiments, the surface coating comprises a fat and/or wax. In some embodiments, the fat and/or wax comprises beeswax, carnauba wax, cetyl alcohol, or cetostearyl alcohol.

In some embodiments, the surface coating comprises a polymer. In some embodiments, the polymer is a hydrophobic polymer, a hydrophilic polymer, a non-fouling polymer, or a combination thereof.

In some embodiments, the hydrophobic polymers is a poly(ester amide), polystyrene-polyisobutylene-polystyrene block copolymer (SIS), polystyrene, polyisobutylene, polycaprolactone (PCL), poly(L-lactide), poly(D,L-lactide), poly(lactides), polylactic acid (PLA), poly(lactide-co-glycolide), poly(glycolide), polyalkylene, polyfluoroalkylene, polyhydroxyalkanoate, poly(3-hydroxybutyrate), poly(4-hydroxybutyrate), poly(3-hydroxyvalerate), poly(3-hydroxybutyrate-co-3-hydroxyvalerate), poly(3-hydroxyhexanoate), poly(4-hyroxyhexanoate), mid-chain polyhydroxyalkanoate, poly (trimethylene carbonate), poly (ortho ester), polyphosphazenes, poly (phosphoester), poly(tyrosine derived arylates), poly(tyrosine derived carbonates), polydimethyloxanone (PDMS), polyvinylidene fluoride (PVDF), polyhexafluoropropylene (HFP), polydimethylsiloxane, poly (vinylidene fluoride-co-hexafluoropropylene) (PVDF-HFP), poly (vinylidene fluoride-co-chlorotrifluoroethylene) (PVDF-CTFE), poly(methacrylates) such as poly(butyl methacrylate) (PBMA) or poly(methyl methacrylate) (PMMA), poly(vinyl acetate), poly(ethylene-co-vinyl acetate), poly(ethylene-co-vinyl alcohol), poly(ester urethanes), poly(ether-urethanes), poly(carbonate-urethanes), poly(silicone-urethanes), poly(urea-urethanes) or a combination thereof.

In some embodiments, the hydrophilic polymer is a polymer or co-polymer of PEG acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), hydroxyl bearing monomers such as HEMA, hydroxypropyl methacrylate (HPMA), hydroxypropylmethacrylamide, and 3-trimethylsilylpropyl methacrylate (TMSPMA), poly(ethylene glycol) (PEG), poly(propylene glycol), SIS-PEG, polystyrene-PEG, polyisobutylene-PEG, PCL-PEG, PLA-PEG, PMMA-PEG, PDMS-PEG, PVDF-PEG, PLURONIC™ surfactants (polypropylene oxide-co-polyethylene glycol), poly(tetramethylene glycol), poly(L-lysine-ethylene glycol) (PLL-g-PEG), poly(L-g-lysine-hyaluronic acid) (PLL-g-HA), poly(L-lysine-g-phosphoryl choline) (PLL-g-PC), poly(L-lysine-g-vinylpyrrolidone) (PLL-g-PVP), poly(109thylamine-g-ethylene glycol) (PEI-g-PEG), poly(109thylamine-g-hyaluronic acid) (PEI-g-HA), poly(109thylamine-g-phosphoryl choline) (PEI-g-PC), and poly(109thylamine-g-vinylpyrrolidone) (PEI-g-PVP), PLL-co-HA, PLL-co-PC, PLL-co-PVP, PEI-co-PEG, PEI-co-HA, PEI-co-PC, and PEI-co-PVP, hydroxy functional poly(vinyl pyrrolidone), polyalkylene oxide, dextran, dextrin, sodium hyaluronate, hyaluronic acid, elastin, chitosan, acrylic sulfate, acrylic sulfonate, acrylic sulfamate, methacrylic sulfate, methacrylic sulfonate, methacrylic sulfamate or a combination thereof.

In some embodiments, the non-fouling polymer is poly(ethylene glycol), poly(alkylene oxide), hydroxyethylmethacrylate (HEMA) polymer and copolymers, poly(n-propylmethacrylamide), sulfonated polystyrene, hyaluronic acid, poly(vinyl alcohol), poly(N-vinyl-2-pyrrolidone), sulfonated dextran, phosphoryl choline, choline, or combinations thereof.

In some embodiments, the polymer comprises a cellulosic polymer, a vinyl polymer, a glycol polymer, an acrylate polymer, or a carbohydrate. In some embodiments, the cellulosic polymer is hydroxypropylmethylcellulose, hydroxypropylcellulose, hydroxyethylcellulose, hydroxyethylcellulose phthalate, ethylcellulose, cellulose acetate phthalate, or cellulose acetate trimellitate. In some embodiments, the vinyl polymer is poly(vinyl pyrrolidone), poly(vinyl alcohol), poly(vinyl pyrrolidone)-poly(vinyl acetate)copolymers, poly(vinyl alcohol)-poly(ethylene glycol) co-polymers, or poly(vinyl acetate phthalate). In some embodiments, the glycol polymer is poly(ethylene glycol). In some embodiments, the acrylate polymer is an amino alkyl methacrylate copolymer. In some embodiments, the carbohydrate is maltodextrin or polydextrose.

In some embodiments, the surface coating comprises a colorant. In some embodiments, the colorant comprises natural pigments, inorganic pigments, water-soluble dyes, FD&C lakes, and D&C lakes. In some embodiments, the natural pigment is riboflavin, beta-carotene, or carmine lake. In some embodiments, the inorganic pigment is titanium dioxide or iron oxides. In some embodiments, the water-soluble dye is FD&C Yellow #5 or FD&C blue #2. In some embodiments, the FD&C lake is FD&C Yellow #5 Lake or FD&C Blue #2 Lake. In some embodiments, the D&C lake is D&C Yellow #10 Lake or D&C Red #30 Lake.

In some embodiments, the surface coating comprises a plasticizer. In some embodiments, the plasticizer is a polyhydric alcohol, acetate ester, phthalate ester, glyceride, oil, or a combination thereof. In some embodiments, the polyhydric alcohol is propylene glycol, glycerol, or polyethylene glycol. In some embodiments, the acetate ester is triacetin, triethyl citrate, or acetyl triethyl citrate. In some embodiments, the phthalate ester is diethyl phthalate. In some embodiments, the glyceride is an acylated monoglyceride. In some embodiments, the oil is castor oil or mineral oil.

In another aspect, the present disclosure provides devices comprising a surface coating provided herein.

In some embodiments, the surface coating is present on at least a portion of an outer surface of the device. In some embodiments, the surface coating is applied in any suitable method including, for example, dip coating and/or spray atomization. Other methods of depositing the coating are also possible. In some embodiments, the surface coating is applied on top of another coating.

In some embodiments, the device is an implantable medical device. In some embodiments, the device is a vascular graft, a stent, a cardiopulmonary bypass circuit, a ventricular assist device, or a respiratory support system. In some embodiments, the device is a vascular graft. In some embodiments, the device is a stent. In some embodiments, the device is a cardiopulmonary bypass circuit. In some embodiments, the device is a ventricular assist device. In some embodiments, the device is a respiratory support system. In some embodiments, the device is a self-expandable stent, balloon-expandable stent, stent-graft, graft (e.g., aortic grafts), artificial heart valve, cerebrospinal fluid shunt, pacemaker electrode, or endocardial lead.

The underlying structure of the device can be of virtually any design. In some embodiments, the device comprises one or more biocompatible materials. In some embodiments, the device comprises a polymer, ceramic, metal, alloy, or a combination thereof. In some embodiments, the metal or alloy is stainless steel, iron-carbon alloy, Field's metal, wolfram, molybdenum, gold, zinc, iron, or titanium. In some embodiments, the metal or alloy is cobalt chromium alloy (ELGILOY), stainless steel (316L), high nitrogen stainless steel, e.g., BIODUR 108, cobalt chrome alloy L-605, “MP35N” (35% cobalt, 35% nickel, 20% chromium, and 10% molybdenum, Standard Press Steel Co., Jenkintown, Pa.), “MP20N” (50% cobalt, 20% nickel, 20% chromium, and 10% molybdenum, Standard Press Steel Co., Jenkintown, Pa.) ELASTINITE (Nitinol), tantalum, nickel-titanium alloy, platinum-iridium alloy, gold, magnesium, or combinations thereof. In some embodiments, the ceramic is hydroxyapatite, aluminum oxide, calcium oxide, tricalcium phosphate, silicates, silicon dioxide, or zirconium oxide. In some embodiments, the polymer is bioabsorbable or biostable. In some embodiments, the polymer is polycaprolactone, polylactic acid, polyethylene glycol, polypropylene, polyethylene, polycarbonate, polystyrene, and polyether ether ketone, or polyvinyl alcohol.

### Methods of Treatment and Uses

Heparin oligomers, oligomeric compounds, polymer conjugates, oligosaccharide conjugates, pharmaceutical compositions, surface coatings, and devices provided herein can be useful for inhibiting or reducing thrombus formation.

In another aspect, the present disclosure provides methods of treating or preventing a disease in a subject in need thereof, comprising administering to the subject an effective amount of a heparin oligomer, oligomeric compound, polymer conjugate, or oligosaccharide conjugate provided herein, or a pharmaceutically acceptable salt, solvate, hydrate, polymorph, co-crystal, tautomer, stereoisomer, isotopically labeled derivative, or prodrug thereof, or a pharmaceutical composition provided herein. In some embodiments, the present disclosure provides methods of treating or preventing a disease in a subject in need thereof, comprising administering to the subject an effective amount of a heparin heptamer provided herein, or a pharmaceutically acceptable salt, solvate, hydrate, polymorph, co-crystal, tautomer, stereoisomer, isotopically labeled derivative, or prodrug thereof, or a pharmaceutical composition provided herein.

In another aspect, the present disclosure provides a use of a heparin oligomer, oligomeric compound, or polymer conjugate provided herein, or a pharmaceutically acceptable salt, solvate, hydrate, polymorph, co-crystal, tautomer, stereoisomer, isotopically labeled derivative, or prodrug thereof, or a pharmaceutical composition provided herein, for the manufacture of a medicament for treating or preventing a disease in a subject in need thereof. In some embodiments, the present disclosure provides a use of a heparin heptamer provided herein, or a pharmaceutically acceptable salt, solvate, hydrate, polymorph, co-crystal, tautomer, stereoisomer, isotopically labeled derivative, or prodrug thereof, or a pharmaceutical composition provided herein, for the manufacture of a medicament for treating or preventing a disease in a subject in need thereof.

In another aspect, the present disclosure provides a heparin oligomer, oligomeric compound, or polymer conjugate provided herein, or a pharmaceutically acceptable salt, solvate, hydrate, polymorph, co-crystal, tautomer, stereoisomer, isotopically labeled derivative, or prodrug thereof, or a pharmaceutical composition for use in treating or preventing a disease in a subject in need thereof. In some embodiments, the present disclosure provides a heparin heptamer, or a pharmaceutically acceptable salt, solvate, hydrate, polymorph, co-crystal, tautomer, stereoisomer, isotopically labeled derivative, or prodrug thereof, or a pharmaceutical composition for use in treating or preventing a disease in a subject in need thereof.

In some embodiments, the effective amount is a therapeutically effective amount. In certain embodiments, a therapeutically effective amount is an amount sufficient for inhibiting thrombus formation. In certain embodiments, a therapeutically effective amount is an amount sufficient for treating a disease. In certain embodiments, a therapeutically effective amount is an amount sufficient for treating cardiovascular disease, atherosclerosis, atherosclerotic lesions, thrombus formation, thromboembolism, stroke, myocardial infarction, or primary or recurrent venous thromboembolism (VTE). In certain embodiments, a therapeutically effective amount is an amount sufficient for reducing thrombus formation and treating a disease. In certain embodiments, a therapeutically effective amount is an amount sufficient for inhibiting thrombus formation and treating a disease. In certain embodiments, a therapeutically effective amount is an amount sufficient for reducing thrombus formation and treating cardiovascular disease, atherosclerosis, atherosclerotic lesions, thrombus formation, thromboembolism, stroke, myocardial infarction, or primary or recurrent venous thromboembolism (VTE). In certain embodiments, a therapeutically effective amount is an amount sufficient for inhibiting thrombus formation and treating cardiovascular disease, atherosclerosis, atherosclerotic lesions, thrombus formation, thromboembolism, stroke, myocardial infarction, or primary or recurrent venous thromboembolism (VTE).

In some embodiments, the effective amount is a prophylactically effective amount. In certain embodiments, a prophylactically effective amount is an amount sufficient for reducing thrombus formation. In certain embodiments, a prophylactically effective amount is an amount sufficient for inhibiting thrombus formation. In certain embodiments, a prophylactically effective amount is an amount sufficient for preventing a disease. In certain embodiments, a prophylactically effective amount is an amount sufficient for preventing cardiovascular disease, atherosclerosis, atherosclerotic lesions, thrombus formation, thromboembolism, stroke, myocardial infarction, or primary or recurrent venous thromboembolism (VTE). In certain embodiments, a prophylactically effective amount is an amount sufficient for reducing thrombus formation and preventing a disease. In certain embodiments, a prophylactically effective amount is an amount sufficient for inhibiting thrombus formation and preventing a disease. In certain embodiments, a prophylactically effective amount is an amount sufficient for reducing thrombus formation and preventing cardiovascular disease, atherosclerosis, atherosclerotic lesions, thrombus formation, thromboembolism, stroke, myocardial infarction, or primary or recurrent venous thromboembolism (VTE). In certain embodiments, a prophylactically effective amount is an amount sufficient for inhibiting thrombus formation and preventing cardiovascular disease, atherosclerosis, atherosclerotic lesions, thrombus formation, thromboembolism, stroke, myocardial infarction, or primary or recurrent venous thromboembolism (VTE).

In some embodiments, the method reduces thrombus formation. In some embodiments, the method reduces thrombus formation by at least about 10%, at least about 15%, at least about 20%, at least about 25%, at least about 30%, at least about 35%, at least about 40%, at least about 50%, at least about 60%, at least about 70%, at least about 80%, at least about 90%, or at least about 95%. In some embodiments, the method reduces thrombus formation by at least about 10%, at least about 15%, at least about 20%, at least about 25%, at least about 30%, at least about 35%, or at least about 40%. In some embodiments, the method reduces thrombus formation by at least about 10%. In some embodiments, the method reduces thrombus formation by at least about 15%. In some embodiments, the method reduces thrombus formation by at least about 20%. In some embodiments, the method reduces thrombus formation by at least about 25%. In some embodiments, the method reduces thrombus formation by at least about 30%. In some embodiments, the method reduces thrombus formation by at least about 35%. In some embodiments, the method reduces thrombus formation by at least about 40%.

In some embodiments, the disease is cardiovascular disease, atherosclerosis, atherosclerotic lesions, thrombus formation, thromboembolism, stroke, or myocardial infarction. In some embodiments, the disease is cardiovascular disease. In some embodiments, the disease is atherosclerosis, atherosclerotic lesions, thrombus formation, thromboembolism, stroke, or myocardial infarction. In some embodiments, the disease is atherosclerosis. In some embodiments, the disease is atherosclerotic lesions. In some embodiments, the disease is thrombus formation. In some embodiments, the disease is thromboembolism. In some embodiments, the disease is stroke. In some embodiments, the disease is myocardial infarction. In some embodiments, the disease is primary or recurrent venous thromboembolism (VTE). In some embodiments, the venous thromboembolism is deep vein thrombosis, pulmonary embolism, or non-occlusive venous thrombosis. In some embodiments, the venous thromboembolism is deep vein thrombosis. In some embodiments, the venous thromboembolism is pulmonary embolism. In some embodiments, the venous thromboembolism is non-occlusive venous thrombosis.

In certain embodiments, the subject is an animal. In certain embodiments, the subject is a human. In certain embodiments, the subject is a human aged 18 years or above. In some embodiments, the subject is a human aged<2 years. In some embodiments, the subject is a human aged 2-6 years, inclusive. In some embodiments, the subject is a human aged 6-18 years, inclusive. In some embodiments, the subject is a human aged 18-65 years, inclusive. In some embodiments, the subject is a human aged>65 years. In certain embodiments, the subject is a non-human animal. In certain embodiments, the subject is a mammal. In certain embodiments, the subject is a non-human mammal. In some embodiments, the subject is a research animal.

In some embodiments, the subject exhibits a prothrombotic phenotype or has elevated heparanase expression, plasma heparanase levels, plasma heparan sulfate concentrations, D-dimer levels, or procoagulant activity. In some embodiments, the subject exhibits a prothrombotic phenotype. In some embodiments, the subject has elevated heparanase expression, plasma heparanase levels, plasma heparan sulfate concentrations, D-dimer levels, or procoagulant activity. In some embodiments, the subject has elevated heparanase expression. In some embodiments, the subject has elevated plasma heparanase levels. In some embodiments, the subject has elevated plasma heparan sulfate concentrations. In some embodiments, the subject has elevated D-dimer levels. In some embodiments, the subject has elevated procoagulant activity.

In some embodiments, the subject has or has been diagnosed with renal insufficiency, type 2 diabetes, a gastrointestinal malignancy, an inflammatory disease, cancer, or a metastatic disease. In some embodiments, the subject has or has been diagnosed with renal insufficiency. In some embodiments, the subject has or has been diagnosed with type 2 diabetes. In some embodiments, the subject has or has been diagnosed with a gastrointestinal malignancy. In some embodiments, the subject has or has been diagnosed with an inflammatory disease. In some embodiments, the inflammatory disease is inflammatory bowel disease, rheumatoid arthritis, or atherosclerosis. In some embodiments, the inflammatory disease is inflammatory bowel disease. In some embodiments, the inflammatory disease is rheumatoid arthritis. In some embodiments, the inflammatory disease is atherosclerosis. In some embodiments, the subject has or has been diagnosed with cancer. In some embodiments, the cancer is lung cancer, breast cancer, colorectal cancer, or pancreatic cancer. In some embodiments, the cancer is lung cancer. In some embodiments, the cancer is breast cancer. In some embodiments, the cancer is colorectal cancer. In some embodiments, the cancer is pancreatic cancer. In some embodiments, the subject has or has been diagnosed with metastatic disease.

In some embodiments, the subject is after surgery, takes oral contraceptives, or has a history of prosthetic valve thrombosis. In some embodiments, the subject is after surgery. In some embodiments, the subject takes oral contraceptives. In some embodiments, the subject has a history of prosthetic valve thrombosis.

In some embodiments, the method further comprises administering an additional therapy or therapeutic agent to the subject before administering the effective amount of the heparin oligomer, oligomeric compound, polymer conjugate or oligosaccharide conjugate. In some embodiments, the method further comprises administering an additional therapy or therapeutic agent to the subject before administering the effective amount of the heparin heptamer. In some embodiments, the method further comprises administering an additional therapy or therapeutic agent to the subject concurrently with administering the effective amount of the heparin oligomer. In some embodiments, the method further comprises administering an additional therapy or therapeutic agent to the subject concurrently with administering the effective amount of the heparin heptamer. In some embodiments, the method further comprises administering an additional therapy or therapeutic agent to the subject after administering the effective amount of the heparin oligomer. In some embodiments, the method further comprises administering an additional therapy or therapeutic agent to the subject after administering the effective amount of the heparin heptamer.

### Methods of Synthesis

In another aspect, the disclosure provides methods of synthesizing a heparin oligomer (e.g., a heparin heptamer) provided herein, comprising the sequential steps of:
(a) elongating a saccharide using:
   (i) recombinant Pasteurella multocida heparosan synthase (pmHS2) and uridine 5-diphospho-N-trifluoroacetyl glucosamine (UDP-GlcNTFA); and
   (ii) recombinant Pasteurella multocida heparosan synthase (pmHS2) and uridine 5-diphospho-N-glucuronic acid (UDP-GlcA)
in either order, one or more times, to obtain a trifluoroacetate-protected oligosaccharide intermediate (e.g., a trifluoroacetate-protected heparin hexamer or trifluoroacetate-protected heparin hexamer-containing intermediate) comprising the structure

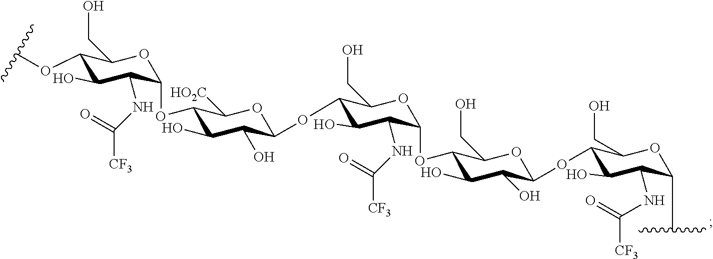

(b) detrifluoroacetylating the oligosaccharide intermediate (e.g., the heparin hexamer oligosaccharide intermediate or heparin hexamer-containing intermediate) of step (a) to obtain an oligosaccharide intermediate (e.g., a heparin hexamer or heparin hexamer-containing oligosaccharide intermediate) comprising the structure

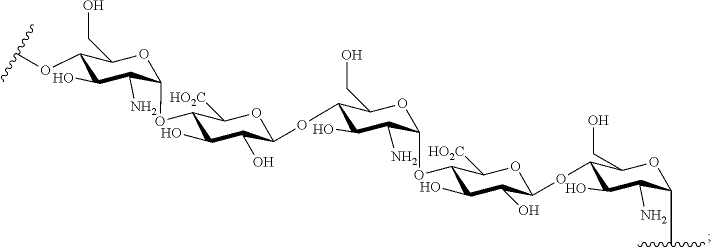

(c) N-sulfating the oligosaccharide intermediate (e.g., the heparin hexamer or heparin hexamer-containing oligosaccharide intermediate) of step (b) to obtain an NS oligosaccharide intermediate (e.g., a NS-hexamer or NS-hexamer-containing oligosaccharide intermediate) comprising the structure

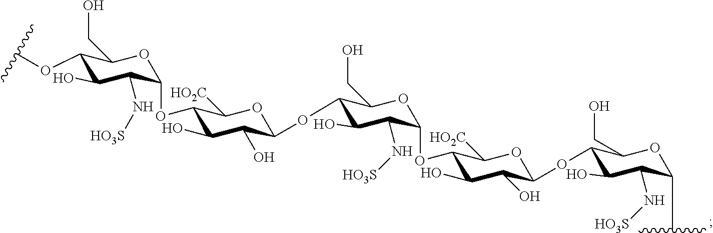

(d) elongating the NS oligosaccharide intermediate (e.g., the NS-hexamer or NS-hexamer-containing intermediate) of step (c) with recombinant Pasteurella multocida heparosan synthase (pmHS2) and uridine 5-diphospho-N-glucuronic acid (UDP-GlcA) to obtain an oligosaccharide intermediate comprising the structure

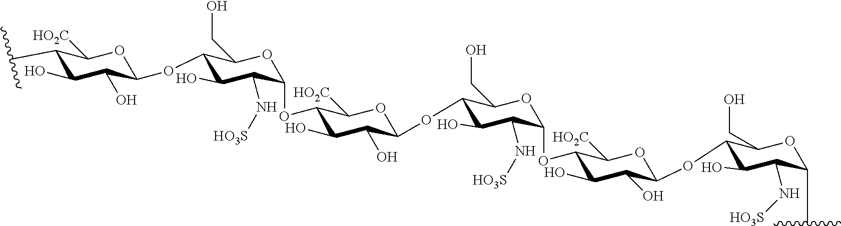

(e) converting glucuronic acid to 2-O-sulfated iduronic acid to obtain an oligosaccharide intermediate comprising the structure

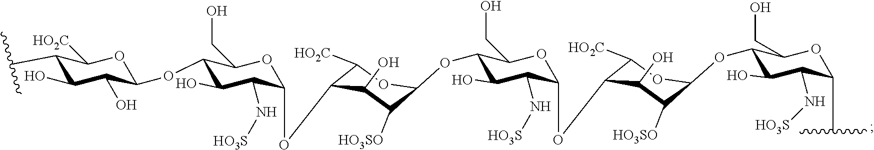

and optionally
(f) performing 3-O- and/or 6-O sulfation of the oligosaccharide intermediate of step (e).

In some embodiments, the saccharide of step (a) is substituted with an aliphatic, heteroaliphatic, carbocyclic, heterocyclic, aryl, or heteroaryl group. In some embodiments, the saccharide of step (a) is an optionally substituted glucuronide. In some embodiments, the saccharide of step (a) is glucuronide substituted with an aliphatic, heteroaliphatic, carbocyclic, heterocyclic, aryl, or heteroaryl group. In some embodiments, the saccharide of step (a) is para-nitrophenyl glucuronide. In some embodiments, the saccharide of step (a) is

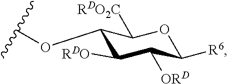

wherein R^{D}, R⁶, and R^{E} are as defined herein.

In some embodiments, the saccharide of step (a) is an optionally substituted glucosamine. In some embodiments, the saccharide of step (a) is a glucosamine substituted with an aliphatic, heteroaliphatic, carbocyclic, heterocyclic, aryl, or heteroaryl group.

In some embodiments, step (b) comprises reaction under basic conditions. In some embodiments, step (b) comprises reaction with a metal hydroxide. In some embodiments, step (b) comprises reaction with lithium hydroxide. In some embodiments, step (b) comprises reaction with sodium hydroxide. In some embodiments, the basic conditions comprise pH of at least 10. In some embodiments, the basic conditions comprise pH of at least 11. In some embodiments, the basic conditions comprise pH of at least 12. In some embodiments, step (b) comprises reaction with base of about 0.05 M. In some embodiments, step (b) comprises reaction with base of about 0.1 M. In some embodiments, step (b) comprises reaction with base of about 0.15 M. In some embodiments, step (b) comprises reaction with base of about 0.2 M.

In some embodiments, step (c) comprises incubation with 3-morpholino-propane-1-sulfonic acid, N-sulfotransferase, and 3′-phosphoadenosine 5′-phosphosulfate. In some embodiments, step (c) is performed at about pH 6.5. In some embodiments, step (c) is performed at about pH 7.0. In some embodiments, step (c) is performed at about pH 7.5.

In some embodiments, step (e) comprises incubation with C₅-epimerase, 2-O-sulfotransferase, and 3′-phosphoadenosine 5′-phosphosulfate in 3-morpholino-propane-1-sulfonic acid buffer. In some embodiments, incubation is performed overnight. In some embodiments, incubation is performed at 37° C.

In some embodiments, step (f) comprises incubation with 3-O-sulfotransferase 3 and 3′-phosphoadenosine 5′-phosphosulfate and/or incubation with 6-sulfotransferase 3 in 3-morpholino-propane-1-sulfonic acid buffer. In some embodiments, incubation is performed overnight. In some embodiments, incubation is performed at 37° C.

In some embodiments, any of steps (a)-(f) is followed by an additional purification step. In some embodiments, each of steps (a)-(f) is followed by an additional purification step. In some embodiments, the additional purification step comprises gel chromatography. In some embodiments, the additional purification step comprises Q-Sepharose column chromatography. In some embodiments, the additional purification step comprises heparin-Sepharose column chromatography.

In some embodiments, the heparin oligomer (e.g., the heparin heptamer) is synthesized in a final yield of about 30% over all steps (a)-(f). In some embodiments, the heparin oligomer (e.g., heparin heptamer) is synthesized in a final yield of about 40% over all steps (a)-(f). In some embodiments, the heparin oligomer (e.g., heparin heptamer) is synthesized in a final yield of about 45% over all steps (a)-(f). In some embodiments, the heparin oligomer (e.g., heparin heptamer) is synthesized in a final yield of about 50% over all steps (a)-(f).

In some embodiments, the heparin oligomer (e.g., heparin heptamer) is synthesized in a final yield of at least 30% over all steps (a)-(f). In some embodiments, the heparin oligomer (e.g., heparin heptamer) is synthesized in a final yield of at least 40% over all steps (a)-(f). In some embodiments, the heparin oligomer (e.g., heparin heptamer) is synthesized in a final yield of at least 45% over all steps (a)-(f). In some embodiments, the heparin oligomer (e.g., heparin heptamer) is synthesized in a final yield of at least 50% over all steps (a)-(f).

In some embodiments, the heparin oligomer (e.g., heparin heptamer) is synthesized in a final yield from about 20% to about 30% over all steps (a)-(f). In some embodiments, the heparin oligomer (e.g., heparin heptamer) is synthesized in a final yield from about 30% to about 40% over all steps (a)-(f). In some embodiments, the heparin oligomer (e.g., heparin heptamer) is synthesized in a final yield from about 35% to about 45% over all steps (a)-(f). In some embodiments, the heparin oligomer (e.g., heparin heptamer) is synthesized in a final yield from about 40% to about 50% over all steps (a)-(f).

In some embodiments, a heparin oligomer (e.g., heparin heptamer) synthesized via a method provided herein can be modified such that the product is a compound of Formula (I). In some embodiments, a heparin oligomer (e.g., heparin heptamer) synthesized via a method provided herein can be chemoenzymatically modified such that the product is a compound of Formula (I). In some embodiments, a heparin oligomer (e.g., heparin heptamer) synthesized via a method provided herein can be chemically modified such that the product is a compound of Formula (I).

In some embodiments, a heparin oligomer (e.g., heparin heptamer) synthesized via a method provided herein can be modified such that the product is a compound of Formula (I-A). In some embodiments, a heparin oligomer (e.g., heparin heptamer) synthesized via a method provided herein can be chemoenzymatically modified such that the product is a compound of Formula (I-A). In some embodiments, a heparin oligomer (e.g., heparin heptamer) synthesized via a method provided herein can be chemically modified such that the product is a compound of Formula (I-A).

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1. Design Of an Ultralow Molecular Weight Heparin that Resists Heparanase Biodegradation

Heparan sulfates (HS) are degraded and depolymerized by heparanase, an endo-P-D-glucuronidase produced by a variety of cells and tissues, including fibroblasts (1), endothelial cells (2), platelets (3), neutrophils (4), activated immune cells (5-7), and primary cancer cells (6, 8, 9). Heparanase displays a range of affinities toward a variety of saccharide motifs (7, 10, 11), but notably cleaves the glycosidic linkage in O-sulfated sequences that contain the antithrombin binding domain (10, 12). The cleavage of HS chains on endothelium by heparanase reduces the local concentration of high affinity antithrombin binding sites with a commensurate effect on blood anticoagulant properties (3). In addition to promoting the degradation of heparan sulfate, heparanase upregulates expression of tissue factor (13) and induces dissociation of tissue factor pathway inhibitor (TFPI) (14). As a consequence, heparanase directly enhances tissue factor activity and FXa production, which further augments cell surface procoagulant activity (15, 16). Significantly, mice overexpressing heparanase exhibit a hypercoagulable phenotype (3) and blood from these mice markedly increase thrombosis on stented arterial segments (17).

Inflammation increases local and systemic levels of heparanase. Heparanase is expressed at sites of local inflammation among patients with inflammatory bowel disease (18), rheumatoid arthritis (19), and atherosclerosis (20). Plasma heparanase activity is significantly elevated among patients with renal insufficiency (21) and type 2 diabetes (22), as well as after surgery (22, 23) and among those on oral contraceptives (24) and with a history of prosthetic valve thrombosis (25). It is particularly noteworthy that tissue and plasma levels of heparanase are also increased in a variety of gastrointestinal malignancies (26-29), as well as among patients with lung cancer (30). All told, elevated activity of heparanase may contribute to the prothrombotic phenotype observed in many of these conditions with a direct correlation between plasma heparan sulfate concentrations, D-dimer levels, and procoagulant activity (31).

Given the increased risk of venous thromboembolism (VTE) among patients with many of these disorders, and especially those with an active cancer, the current standard of care is thromboprophylaxis with anticoagulation. However, both unfractionated and low molecular weight heparin (LMWH) are susceptible to cleavage by heparanase with neutralization of anticoagulant properties (32, 33). Perhaps not surprisingly, meta-analyses and randomized controlled trials demonstrate that LMWH provides incomplete protection from cancer associated VTE (34, 35). Moreover, while newer direct oral anticoagulants (DOACs), such as direct FXa inhibitors display similar efficacy to LMWH, they have been associated with an increased incidence of major bleeding events among patients with cancer (36). The more limited effectiveness of heparin in patients with malignancies and among those with a variety of underlying inflammatory disorders may be attributed, in part, to high levels of circulating heparanase.

Heparin and LMWH are currently sourced from porcine mucosa, but recent efforts have led to the development of novel, scalable, chemoenzymatic schemes for the synthesis of a variety of N-sulfo heparin oligosaccharides with therapeutic potential (37). For example, as a structural analogue of fondaparinux (Arixtra™), a heptasaccharide with comparable anti-FXa activity was synthesized in far fewer steps and much higher overall yield (45% vs. 0.01%). A 21-mer oligosaccharide was also synthesized and exhibits both anti-FXa and anti-IIa activity, comparable to enoxaparin and unfractionated heparin, but with very low binding toward platelet factor 4, thereby, limiting the risk of heparin-induced thrombocytopenia (38). Significantly, the synthesis of structurally defined heparan sulfate oligosaccharides has also provided important insights into the substrate specificity of heparanase (39-41). In particular, these studies led to the discovery that heparanase cleaves the glycosidic linkage between glucuronic acid (GlcA) and an N-sulfo glucosamine unit carrying either a 3-O- or a 6-O-sulfo group (39). This heparanase sensitive linkage is typified by the disaccharide -[GlcA-GlcNS3S6S]—, which is found within the antithrombin binding pentasaccharide (42). Heparanase displays different cleavage modes by recognizing structures at the non-reducing end of the substrate, presumably through allosteric regulation (40, 41). Given that only a single heparanase isoform has been discovered, the existing view is that heparanase is capable of degrading a broad range of structurally diverse HS polysaccharides. Nevertheless, a knowledge of the discrete substrate specificities for heparanase has enabled the design and synthesis of a heparanase-resistant, ultralow molecular weight heparin without an internal GlcA residue, but with intact anti-FXa activity, as reported herein.

### Materials

All reagents were purchased from Sigma-Aldrich and used without further purification unless otherwise specified.

### Chemoenzymatic Synthesis of Heparin Oligosaccharides

The expression and purification of all enzymes used in the synthesis of heparin oligosaccharides are detailed elsewhere (43, 44). In brief, synthesis of a 6-mer oligosaccharide intermediate was initiated through elongation of commercially available para-nitrophenyl glucuronide (GlcA-pNP, Carbosynth) using recombinant Pasteurella multocida heparosan synthase (pmHS2) and uridine diphosphate sugar nucleotide donors, UDP-GlcNTFA (uridine 5-diphospho-N-trifluoroacetyl glucosamine) and UDP-GlcA (uridine 5-diphospho-N-glucuronic acid). Once the oligosaccharide was extended to six saccharide units, the product was de-trifluroacetylated by addition of 0.1 M LiOH, maintaining pH above 12 for 10 min at room temperature. The formation of the detrifluoroacetylated hexasaccharide was monitored by ESI-MS. The compound was then added to a solution containing 50 mM MOPS (3-morpholino-propane-1-sulfonic acid), 32 μg/mL N-sulfotransferase, and PAPS (3′-phosphoadenosine 5′-phosphosulfate, 1.5 molar equivalents of free amino groups) at pH 7.0 and incubated overnight at 37° C. (45). The NS-6-mer intermediate was purified by gel chromatography using a Q-Sepharose column and aliquoted to prepare the 7-mer and 6-mer heparin oligosaccharides.

Synthesis of a heparanase resistant 7-mer heparin oligosaccharide (HR 7-mer) was achieved by elongation of the NS-6-mer intermediate with GlcA. The heptasaccharide (1 mg/mL) was subsequently modified by overnight incubation at 37° C. with C5-epimerase (0.004 mg/mL), 2-O-sulfotransferase (0.01 mg/mL), and PAPS (3′-phosphoadenosine 5′-phosphosulfate) (1 mg/mL) in 50 mM MOPS buffer (pH 7.0) to convert the GlcA to a 2-O-sulfated iduronic acid. The NS2S 7-mer intermediate (GlcA-GlcNS-IdoA2S-GlcNS-IdoA2S-GlcNS-GlcA-pNP) was subsequently purified by Q-Sepharose column chromatography. The intermediate (1 mg/mL) was then incubated overnight at 37° C. with 3-O-sulfotransferase 3 (3-OST-3; 0.05 mg/mL) and PAPS (1 mg/mL), ensuring 3-O-sulfation, followed by Q-Sepharose column purification. The intermediate (1 mg/mL) was then incubated with 6-OST-3 (0.15 mg/mL) in 50 mM MOPS buffer pH 7.0 and PAPS (2 mg/mL) to install 6-O-sulfation. The final 7-mer product was purified by Q-Sepharose column chromatography, followed by purity analysis using HPLC and molecular weight determination by ESI-MS.

Synthesis of the heparanase sensitive heparin 6-mer oligosaccharide (HS 6-mer), as a structural analogue of fondaparinux, was achieved by overnight incubation of the NS-6-mer intermediate (1 mg/mL) at 37° C. in the presence of C5-epimerase (0.004 mg/mL), 2-O-sulfotransferase (0.01 mg/mL), and PAPS (0.5 mg/mL) in 50 mM MOPS buffer (pH 7.0) to convert the GlcA to a 2-O-sulfated iduronic acid. The NS2S 6-mer intermediate (GlcNS-IdoA2S-GlcNS-IdoA2S-GlcNS-GlcA-pNP) was subsequently purified by Q-Sepharose column chromatography. The product (1 mg/mL) was then incubated with 6-OST-3 (0.1 mg/mL) in 50 mM MOPS buffer pH 7.0 and PAPS (1.5 mg/mL) to enable 6-O-sulfation, followed by overnight incubation with 3-OST-1 (0.01 mg/mL) and PAPS (0.5 mg/mL) at 37° C., ensuring 3-O-sulfation. The final 6-mer product was purified over a Q-Sepharose column, followed by purity analysis using HPLC, and molecular weight determined by ESI-MS.

Incubation of Heparin Oligosaccharides with Recombinant Heparanase

Recombinant heparanase was expressed in insect cells using the baculovirus expression system and purified by a heparin-Sepharose column, as previously described (40). Heparin oligosaccharides, HS 6-mer (20 μg) or HR 7-mer (20 μg), were incubated overnight at 37° C. in 250 μL of MES (50 mM, pH 6.0) in the presence of heparanase (2 μg for 1× heparanase digestion, or 20 μg for 10× heparanase digestion).

### High Performance Liquid Chromatography

High performance liquid chromatography (HPLC) analysis was conducted with a strong anion exchange column (Propac™ PA1 column, 10 μm, 9 mm×250 mm, Thermo Fisher, Waltham, MA) using a Shimadzu Prominence UFLC20A system (Shimadzu Corporation, Columbia, MD) with a UV detector set at 310 nm. A linear gradient was used for analytical purposes and included a solvent A: 20 mM NaAcO (pH 5.0); and solvent B: 2 M NaCl with 20 mM NaAcO (pH 5.0): 0-3 min: 0-80% B, 3.1-23 min: 80-100% B at a flow rate of 2 mL/min. A total of 5-10 μg of oligosaccharides was injected per run.

### Electrospray Ionization Mass Spectrometry

ESI-MS analysis was performed using a Thermo LCQ-Deca mass spectrometer (Thermo Fisher, Waltham, MA) in negative ionization mode. A syringe pump (Harvard Apparatus) was used to introduce the sample by direct infusion (50 L/min). A total of 2 to 5 μg of sample was diluted in 200 L of 10 mM ammonium bicarbonate with the electrospray source set to 3 kV and 150° C. The automatic gain control was set to 1×107 for a full scan MS and the MS data was acquired and processed using Xcalibur 1.3.

Liquid Chromatography with Tandem Mass Spectrometry

LC-MS/MS analysis of heparanse digested oligosaccharides was implemented on a Vanquish Flex UHPLC system (Thermo Fisher, Waltham, MA) coupled with a TSQ Fortis triple-quadrupole mass spectrometer. The ACQUITY Glycan BEH Amide column (1.7 m, 2.1×150 mm; Waters) was used at 60° C. The mobile phase A was 50 mM ammonium formate in water (pH 4.4) and mobile phase B was acetonitrile. The elution gradient and flow rate consisted of: 0-6 min 83% B, flow rate 0.3 mL/min; 6.1-45 min 83-5% B, flow rate 0.25 mL/min; 45-55 min 5% B, 0.25 mL/min; 55.1-65 min 83% B, flow rate 0.3 mL/min. On-line triple-quadrupole mass spectrometry was used as the detector. ESI-MS analysis was operated in the negative-ion mode using the following parameters: Negative ion spray voltage at 3.0 kV, sheath gas at 55 Arb, aux gas 25 arb, ion transfer tube temperature at 250° C. and vaporizer temperature at 400° C. TraceFinder software was applied for data processing. A total of 0.5 to 1 μg of heparanase digested oligosaccharides in 2 μL was injected per run.

Anti-FXa activity

Human FXa (Enzyme Research Laboratories, South Bend, Indiana, United States of America) was diluted in phosphate buffered saline (PBS) to 50 U/mL. Antithrombin was diluted to 0.03 mg/mL in PBS with 1 mg/mL of bovine serum albumin (BSA). The chromogenic substrate, S-2765 (Diapharma, Westchester, Ohio, United States of America) was diluted to 1 mg/mL in water. Heparin oligosaccharides were dissolved in PBS at various concentrations (7 to 3600 ng/mL). A reaction mixture including 60 μL of antithrombin and 24 μL of a solution containing a heparin oligosaccharide was incubated at room temperature for 3 min. FXa (100 μL) was then added, which was followed after a 2 min incubation at room temperature by the addition of 30 μL of chromogenic substrate, S-2765. The absorbance of the mixture was measured at 405 nm for 5 min and plotted against reaction time. The initial reaction rate as a function of oligosaccharide concentration was used to calculate IC₅₀.

### Animals

C57BL/6 mice (22-25 g, 8 weeks old, male, Jackson Laboratory, Bar Harbor, Maine, United States of America) were used for all animal experiments. Protocols were approved by the Institutional Animal Care and Use Committee of Beth Israel Deaconess Medical Center.

### Pharmacokinetic/Pharmacodynamic Analysis

Sample collection was initiated 1 hour after subcutaneous administration of HR 7-mer (12 μg/g) to C57BL/6 mice. Blood samples (~200 μL) were collected into EDTA coated microtubes by cheek puncture from the submandibular vein at 15, 30, 60, 120, and 180 min. Platelet-rich plasma (PRP) was isolated by centrifugation of blood at 150 g for 10 min at room temperature and platelet-poor plasma (PPP) obtained by centrifugation of PRP at 1,200 g for 5 min at room temperature. Plasma was used immediately used or stored at −80° C. Plasma anti-FXa activity was assessed using the ACTICHROME heparin (anti-FXa) activity assay (BioMedica Diagnostics, Windsor, Ontario) and reported as U/mL using an enoxaparin generated standard curve.

### Murine Model of Non-Occlusive Venous Thrombosis

Heparin-based drug efficacy was evaluated in a preclinical mouse model of non-occlusive venous thrombosis (46). HR 7-mer was administered subcutaneously (12 μg/g, n=5) to male C57BL/6 mice (8-12 weeks of age) 1 hour prior to electrolytic injury and 24 hours after injury. Enoxaparin was administered subcutaneously (4 μg/g, n=5) 4 hours prior and 24 hours after electrolytic injury as a clinically relevant reference and an additional control group enrolled with saline vehicle administered subcutaneously (n=6). Non-occlusive venous thrombosis was induced by electrolytic injury of the inferior vena cava. Mice were anesthetized with 2% isoflurane and the inferior vena cava approached via a midline laparotomy. Venous side branches were ligated or cauterized, while posterior branches were left patent. A 25-gauge stainless steel needle, attached to a silver-coated copper wire was inserted into the exposed caudal vena cava and positioned against the anterior wall (anode). A second wire was implanted subcutaneously to complete the circuit (cathode) and a 250 μAmps current applied for 15 min. Subsequently, the needle was removed and a cotton swab held in gentle contact with the puncture site to prevent bleeding. The vena cava and associated thrombus, immediately below the renal veins to just above the bifurcation, was excised 48 hours after injury for determination of wet thrombus weight.

### Tail Vein Transection Bleeding Time

The effect of test drug on bleeding time was assessed in a murine tail vein transection model (46). Sterile saline (n=5), HR 7-mer (12 μg/g, n=5) or enoxaparin (4 μg/g, n=7) was administered subcutaneously. Mice were anesthetized with ketamine and xylazine by intraperitoneal injection and placed on a warming mat at 37° C. Either 1 hour or 4 hours after administration of HR 7-mer or enoxaparin, respectively, the lateral tail vein was transected with a number 11 scalpel blade at a tail width of 2.3 mm and immediately submerged in PBS at 37° C. The bleeding time was determined at that instant when bleeding had ceased for 30 seconds. Animals were excluded if arterial bleeding was present.

### Statistical Analysis

Descriptive data are presented as mean±SEM unless otherwise stated. Group comparisons were conducted using the unpaired Student's t-test. Significance was indicated as *p≤0.05, **p≤0.01, and ***p≤0.001 and values of p≤0.05 were considered statistically significant.

### Results

### Chemoenzymatic Synthesis of a Heparanase-Resistant Ultralow Molecular Weight Heparin

A variety of structurally homogeneous heparin analogues have been synthesized with well-defined patterns of N- and O-sulfation through the introduction of new chemoenzymatic schemes based on the use of para-nitrophenyl derivatized glucuronic acid (GlcA-p-NP), natural and unnatural nucleotide sugar donors, glycosyltransferases, sulfotransferases, and an epimerase (37, 43, 45). In brief, these methodologies have established that synthetic heparins can be generated through initial backbone elongation of sugar acceptors followed by chemoenzymatic modifications of the oligosaccharide intermediate (47). GlcA-p-NP is a particularly convenient sugar acceptor that exhibits strong ultraviolet absorbance and facilitates oligosaccharide detection and purification. The p-NP group can be removed by oxidative cleavage or used to introduce an azide or amino group for a subsequent conjugation reaction (48). Backbone elongation is achieved by transferring uridine diphosphate (UDP) monosaccharide donors to the glycosyl acceptor through a series of glycosyltransferase reactions. Typically, backbone elongation has been mediated by a recombinant bacterial glycosyltransferase, such as PmHS2 (49). While UDP-GlcA and UDP-GlcNAc represent the natural donor substrates used for heparin biosynthesis, given the absence of an efficient recombinant N-deacetylase/N-sulfotransferase (NDST), the production of heparin analogues containing GlcNS has been challenging. To address this shortcoming, UDP-GlcNTFA was used as a means to incorporate GlcNS residues at precise positions (37, 50). Exposure to mild alkaline conditions removed the trifluroacetyl group, and the residue was then N-sulfated by an N-sulfotransferase.

Adopting these approaches, an ultralow molecular weight heparin was designed that displays anti-FXa activity that is not susceptible to inactivation by heparanase. A heparin heptasaccharide (HR 7-mer) was synthesized in which the glycosidic linkage -[GlcA-GlcNS3S6S]- within the antithrombin binding domain was replaced by -[IdoA2S-GlcNS3S6S]-. As a reference compound, a heparin hexasaccharide (HS 6-mer) was also synthesized, which apart from the presence of GlcA-pNP at the reducing end, contained the canonical, heparanase sensitive, -[GlcA-GlcNS3S6S]- disaccharide and was otherwise identical to fondaparinux.

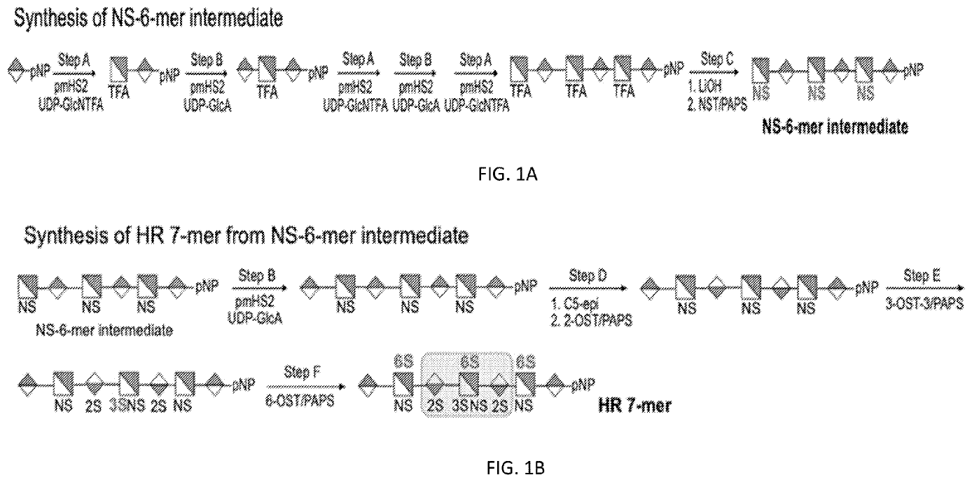

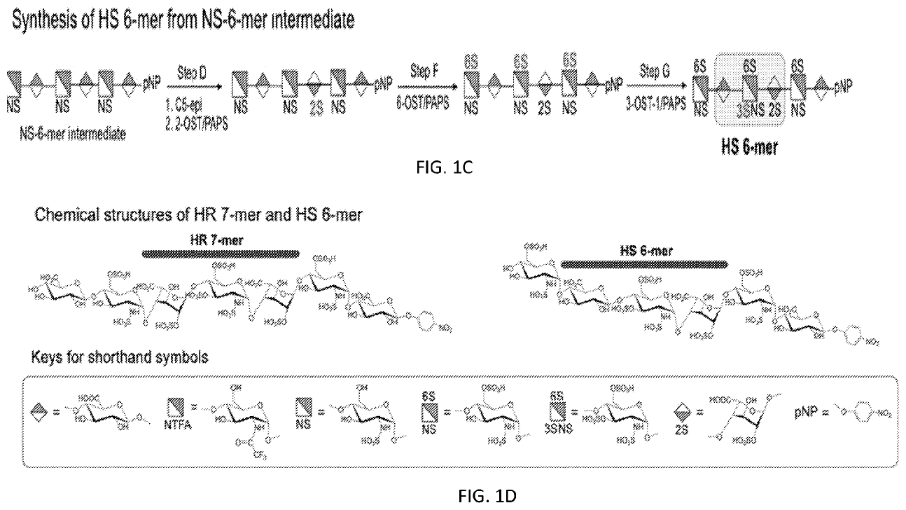

Synthesis of both HR 7-mer and HS 6-mer was initiated using GlcA-pNP as an acceptor to afford an NS-6-mer intermediate in six enzymatic steps (FIG. 1A). This intermediate was then converted to either HR 7-mer in four steps (FIG. 1B) or HS 6-mer in three steps (FIG. 1C). As noted, HR 7-mer was designed with GlcNS3S6S flanked by two IdoA2S residues, while HS 6-mer contained the naturally occurring antithrombin binding domain in which GlcNS3S6S is flanked by GlcA and IdoA2S (FIG. 1D). Without wishing to be bound by any particular theory, it was postulated that this structural difference would limit the ability of heparanase to cleave the antithrombin binding domain but otherwise preserve antithrombin binding and anti-FXa activity.

### HR 7-Mer Displays Preserved Anti-FXa Activity in the Presence of Heparanase

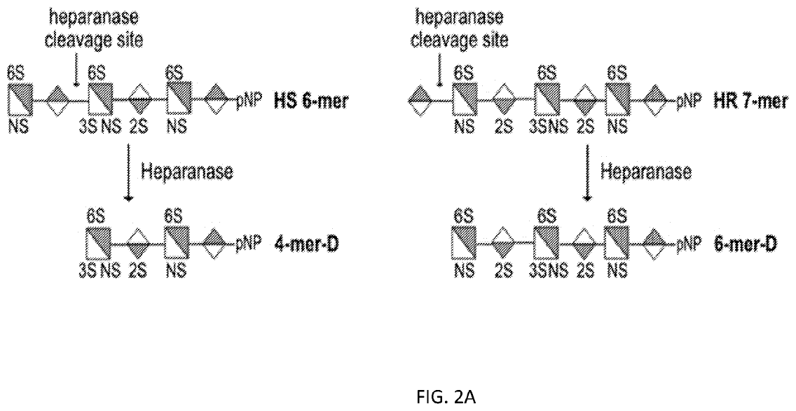

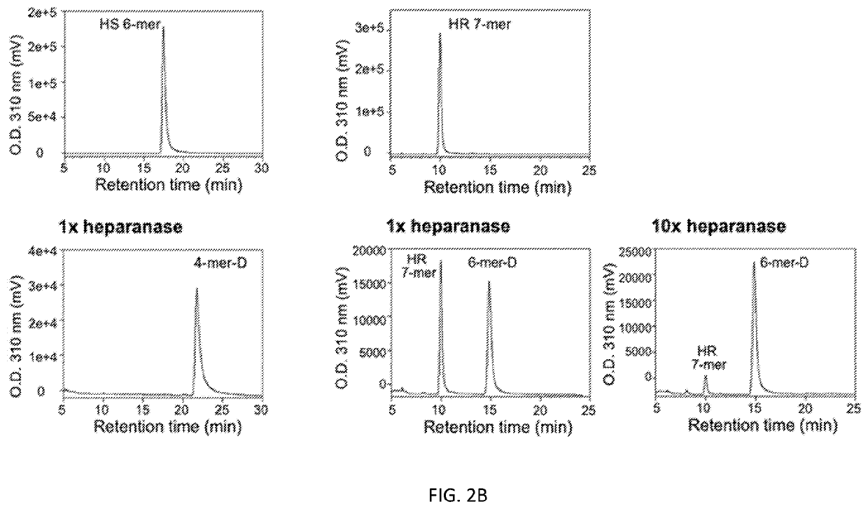

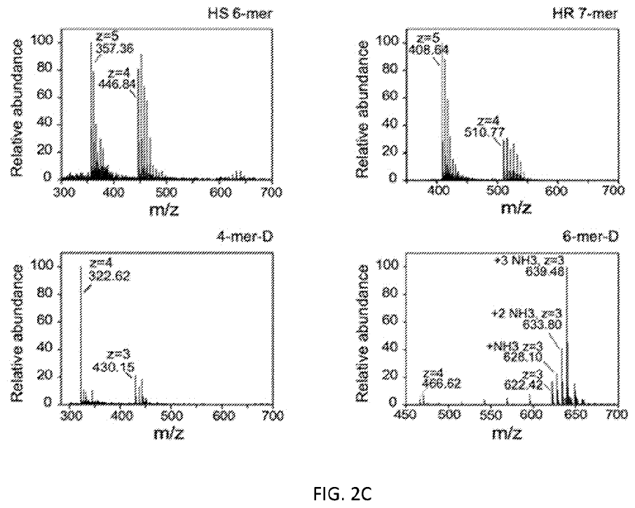

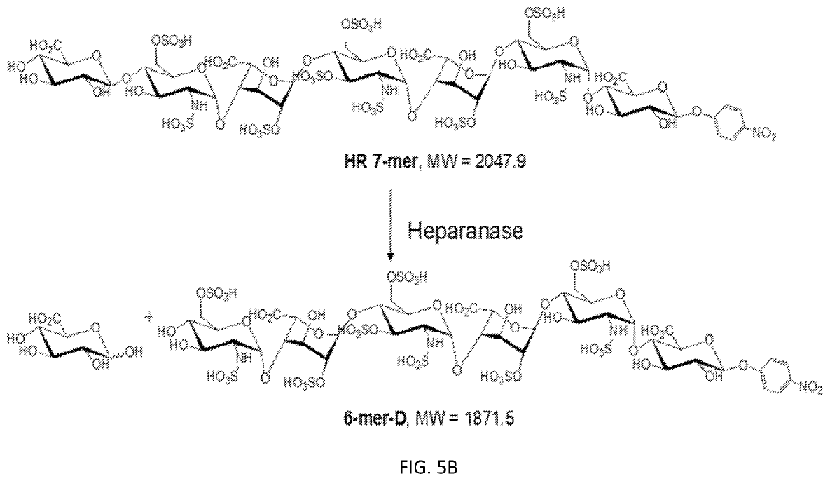

The susceptibility of HS 6-mer and HR 7-mer to heparanase mediated degradation was initially evaluated by HPLC and LC-MS (FIG. 2A). Overnight incubation of HS 6-mer with heparanase resulted in an increase in HPLC retention time from 17 to 22 min (FIG. 2B) with LC-MS analysis confirming a reduction in molecular weight from 1791.4 to 1294.0 Da, consistent with cleavage of the -[GlcA-GlcNS3S6S]- disaccharide to afford a tetrasaccharide (4-mer-D) (FIG. 2B, FIG. 5A). HR 7-mer contained a potential heparanse cleavage site, -[GlcA-GlcNS6S]-, located outside of the pentasaccharide antithrombin binding domain. Incubation of HR 7-mer with heparanase resulted in an increase in HPLC retention time from 10 to 15 min with LC-MS analysis confirming the loss of GlcA with a reduction in molecular weight from 2047.9 to 1871.5 to afford a hexasaccharide (6-mer-D) (FIG. 2C, FIG. 5B). As an endolytic enzyme, heparanase has an established preference for GlcA-containing glycosidic linkages in the middle of a saccharide substrate (40). These studies are consistent with this observation, as cleavage of the glycosidic linkage between GlcA and GlcNS6S at the non-reducing end of HR 7-mer required sustained incubation at high concentrations of heparanase.

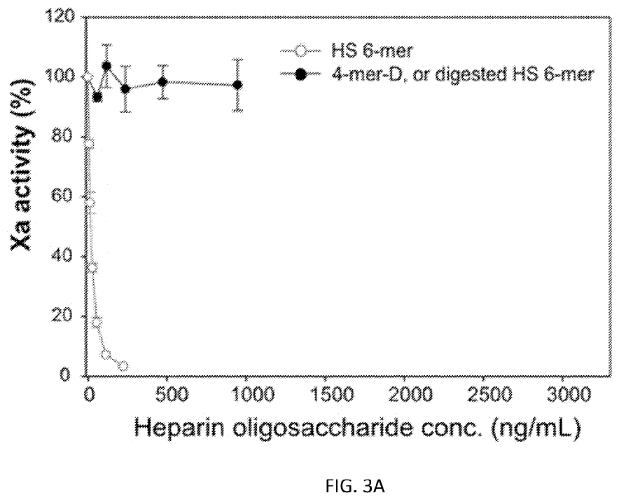

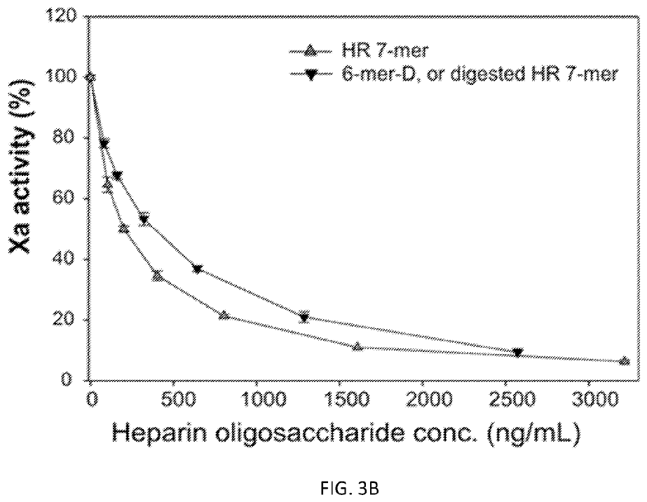

Both HS 6-mer and HR 7-mer displayed potent anti-FXa activity with a calculated IC₅₀ of 38 ng/mL (21 nM) and 196 ng/mL (96 nM), respectively (FIGS. 3A and 3B). Anti-FXa activity was completely lost after HS 6-mer was incubated with heparanase, with no measurable activity observed for the digested byproduct (4-mer-D) (FIG. 3A). In contrast, anti-FXa activity was largely preserved for HR 7-mer, with the byproduct (6-mer-D) exhibiting an IC₅₀ of 362 ng/mL (193 nM) (FIG. 3B).

### An Ultralow Molecular Weight Heparanase-Resistant Heparin Inhibits Venous Thrombosis

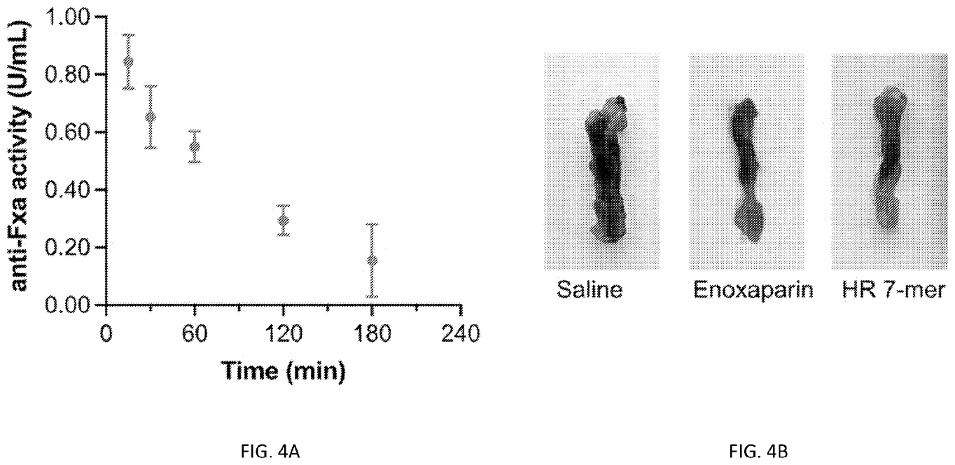

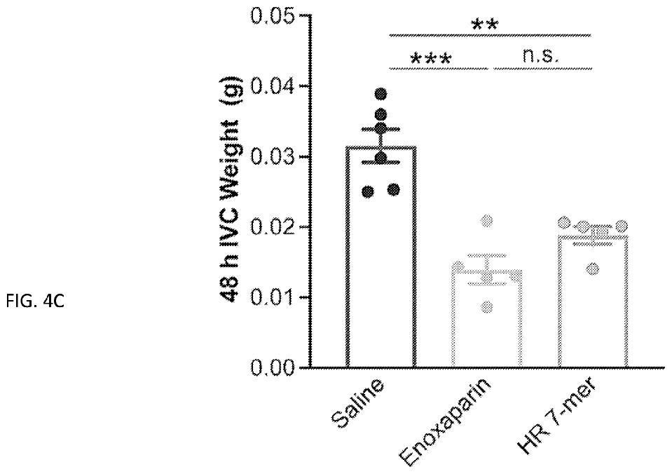

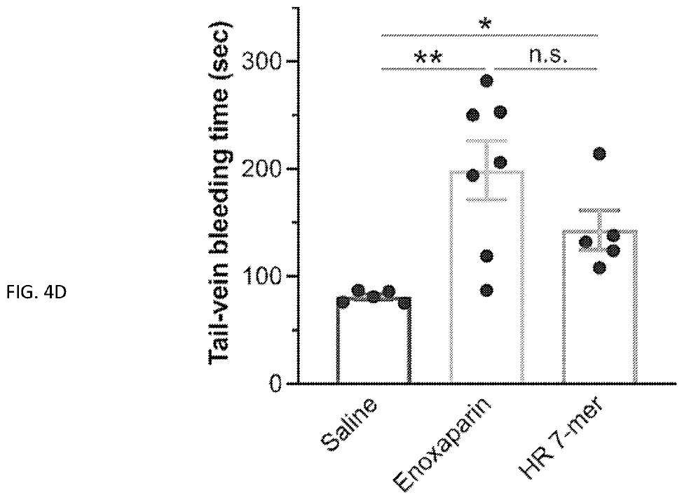

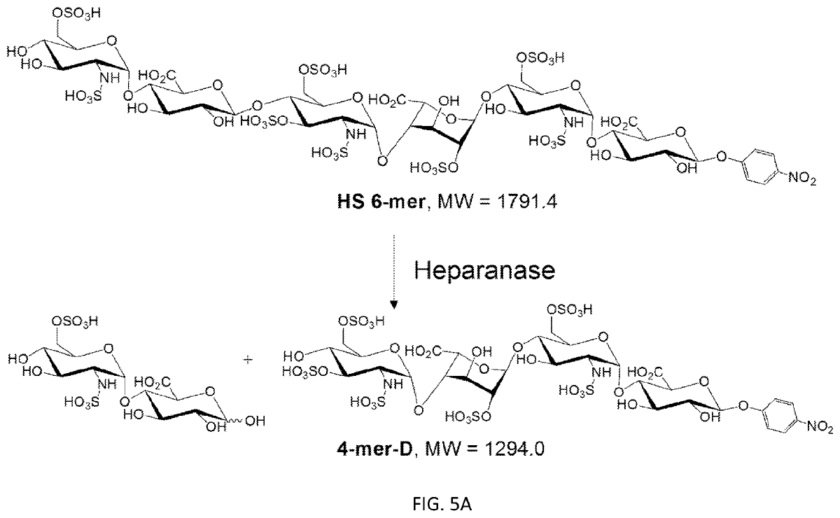

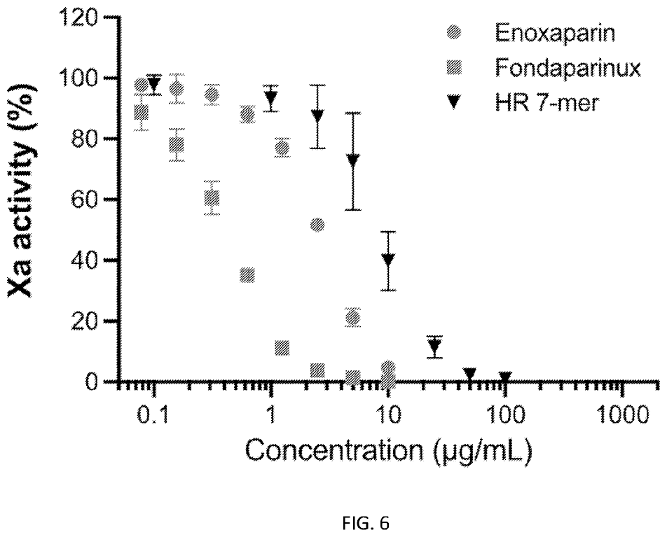

The effectiveness of HR 7-mer as a heparin-based drug for venous thromboprophylaxis was evaluated in a murine model of non-occlusive venous thrombosis. Enoxaparin was used as a conventional reference standard and was dosed according to recommended thromboprophylaxis guidelines to achieve a plasma anti-FXa activity of 0.4 to 0.6 U/mL (51). In this regard, subcutaneous administration of enoxaparin (4 μg/g) resulted in a plasma anti-FXa activity of 0.54 U/mL four hours after subcutaneous dosing (n=3, data not shown), consistent with prior studies (46). On a weight basis, the anti-FXa activity of HR 7-mer was three-fold lower than enoxaparin (FIG. 6). Therefore, HR 7-mer was administered at a three-fold higher dose than enoxaparin (12 μg/g SC) and plasma anti-FXa activity measured over a 3 hour time period (FIG. 4A). A plasma anti-FXa activity of 0.55 U/mL was observed 1 hour after administration of HR 7-mer and, as a consequence, this dose and time point was selected for in vivo studies of venous thromboprophylaxis and bleeding time.

HR 7-mer (12 μg/g, SC) and enoxaparin (4 μg/g, SC) were administered 1 hour or 4 hours, respectively, prior to electrolytic injury of the vena cava and 24 hours thereafter. Administration of HR 7-mer led to a 40.3±3.9% reduction in thrombus formation, as compared to saline vehicle, which was similar to that observed for enoxaparin (55.8±6.3%) (FIGS. 4B and 4C). As compared to the observed bleeding time for saline vehicle (81±3 sec), administration of enoxaparin (199±27 sec) and HR 7-mer (143±18 sec) led to comparable increases (FIG. 4D). The antithrombotic and bleeding profile of enoxaparin and HR 7-mer are summarized in Table 1.

TABLE 1
Summary of anticoagulant properties of HR 7-mer

| | Saline | | Enoxaparin | | HR 7-mer | |
|---|---|---|---|---|---|---|
| Dosage | n/a | | 4 μg/g | | 12 μg/g | |
| Administration | n/a | | 4 hr pre, SC | | 1 hr pre, SC | |
| Plasma anti-FXa activity (IU/ml) | n/a | | 0.54 ± 0.09 | (n = 4) | 0.55 ± 0.05 | (n = 5) |
| Avg. IVC weight (mg) | 31.5 ± 2.3 | (n = 6) | 13.9 ± 2.0 | (n = 5) | 18.8 ± 1.2 | (n = 5) |
| Avg. bleeding time (sec) | 81 ± 3 | (n = 5) | 199 ± 27 | (n = 7) | 143 ± 18 | (n = 5) |

### Discussion

Heparanase cleaves the glycosidic linkage between glucuronic acid (GlcA) and a 3-O- or 6-O-sulfated glucosamine (40), typified by the disaccharide -[GlcA-GlcNS3S6S]-, which is found within the antithrombin binding domain of all heparan sulfates and heparins (42). As a consequence, heparanase mediated degradation of heparan sulfates and heparin leads to loss of anticoagulant activity (3, 10, 12, 32, 33). Thus, apart from the ability of heparanase to promote a thrombotic phenotype by enhancing tissue factor activity and FXa generation, as well as through the cleavage of endogenous heparan sulfates, high circulating levels of heparanase may also limit the clinical effectiveness of unfractionated heparin, low molecular weight heparin, as well as the ultralow molecular weight synthetic heparin, fondaparinux. Indeed, the exposure of fondaparinux to plasma heparanase is substantial with an elimination half-life of 17 to 21 hours in healthy adults, which is further prolonged among those over 75 years of age or among individuals weighing less than 50 kg or with renal insufficiency. While eliminated more rapidly, the half-life of enoxaparin after repeated dosing is 7 hours. All told, the decline of anti-FXa activity following subcutaneous administration of either a low molecular weight or ultralow molecular weight heparin may be substantially accelerated among subsets of patients with high circulating levels of heparanase, such as those with cancer.

There is an urgent need for more effective therapies to reduce the risk of primary and recurrent VTE among cancer patients. As the standard of care, both low molecular weight heparin and direct oral anticoagulants are limited by incomplete protection and a significantly increased risk of major bleeding (34-36). Dalteparin (Fragmin) is the only FDA approved anticoagulant for prevention of recurrent VTE in cancer patients, but carries an FDA black box warning for risk of epidural or spinal hematoma and has also been associated with a risk of thrombocytopenia that may lead to either terminating or reducing the dose in a significant proportion of patients. VTE occurs in approximately 20% of all cancer patients (52) with a 4- to 7-fold increased risk as compared to those without cancer and a 20-fold increased risk for those patients with metastatic disease (53). Once VTE occurs, cancer patients have a three- to four-fold higher rate of VTE recurrence compared to patients without cancer (54). Significantly, VTE occurs disproportionately in a number of specific types of cancers, including lung, breast, colorectal, and pancreatic cancer; all of which are associated with increased heparanase expression and elevated levels of plasma heparanase (55).

As a second major need, the clinical benefit of anti-thrombogenic heparinized surface coatings has been observed for a variety of blood contacting devices, including heparin-coated vascular grafts (56-58), heparin-bonded PTFE covered stents (59, 60), cardiopulmonary bypass circuits (61), as well as ventricular assist devices (62) and respiratory support systems (63). Nonetheless, the early clinical benefit of heparinized coatings is not maintained, presumably due to the inability to sustain heparin activity (58, 64-67). Notably, the few claims of persistent heparin activity on explanted heparin-bonded devices have been based on the measurement of antithrombin binding, without distinguishing high affinity binding sites necessary for the conformational activation of antithrombin from low affinity sites, or determining the anti-FXa or anti-IIa activity of the surface coating (68, 69).

To this end, a non-natural ultralow molecular weight heparin heptasaccharide (HR 7-mer) was synthesized. The HR 7-mer does not contain an internal GlcA residue, but otherwise displays anticoagulant activity. Specifically, a chemoenzymatic scheme was developed using a glycosyl transferase (pmHS2), an epimerase (C5-epi), and four distinct sulfotransferases, including NST, 2-OST, 3-OST-3 and 6-OSTs, which replaced -[GlcA-GlcNS3S6S]- with -[IdoA2S-GlcNS3S6S]- (Residue 3-4). To generate the unique disaccharide sequence in HR 7-mer, a 3-O-sulfotransferase isoform 3 (3-OST-3) was used following a newly discovered enzymatic modification sequence (44). This structural difference within HR 7-mer was found to be critical for resistance to heparanase digestion. While a GlcA residue is present at the non-reducing end of HR 7-mer, this site is relatively inaccessible to heparanase digestion since the enzyme only cleaves internal GlcA-GlcNS6S linkages as revealed by our prior studies of substrate specificity (40). The anti-FXa activity of a native structural analogue of the heparin pentasaccharide that defines fondaparinux was completely lost after heparanase digestion, consistent with the known susceptibility of native heparin. In contrast, the anticoagulant activity of HR 7-mer was unaffected. These results were also confirmed by HPLC and ESI-MS analysis. Importantly, HR 7-mer and related analogues were produced on gram-scale.

### Example 2. Design, Synthesis, and Activity of an Oligosaccharide Conjugate Comprising a Heparanase-Resistant Heparin Oligomer Domain and a Second Oligosaccharide Domain

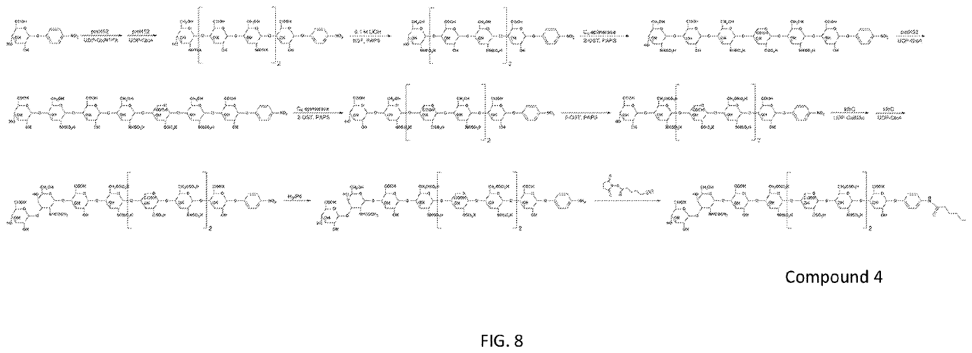

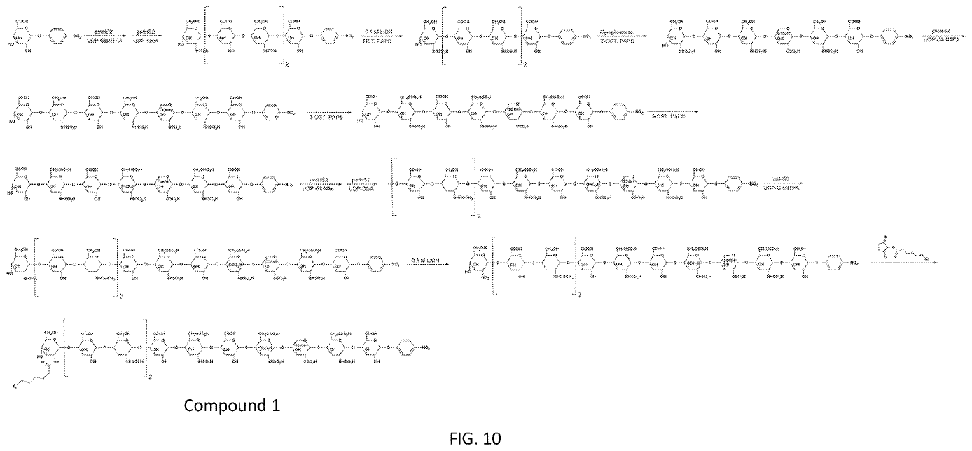

A conjugate comprising a first domain comprising a HR heparin oligomer and a second domain comprising another oligosaccharide (e.g., comprising an anti-FIIa pentasaccharide moiety) was prepared using Click chemistry. More particularly, an azide-functionalized oligosaccharide (Compound 3) comprising a HR heparin oligomer was prepared as shown in FIG. 7. An alkyne-functionalized oligosaccharide (Compound 4) comprising a pentasaccharide with anti-FIIa activity was prepared as shown in FIG. 8. Conjugate 6 was provided via a copper-catalyzed azide-alkyne cycloaddition (CuAAC) of Compounds 3 and 4. See FIG. 9. To enhance the heparanase-resistance of the conjugate, galactosamine residues were also included in both Compounds 3 and 4. For comparison, a second conjugate (Compound 5) was prepared from an azide-functionalized oligosaccharide (Compound 1) comprising a HS heparin oligomer and an alternative alkyne-functionalized oligosaccharide (Compound 2). See FIGS. 10-12.

Chemoenzymatic Synthesis of functionalized Oligosaccharides

Compound 3: As shown in FIG. 7, the elongation of the HS (Heparan Sulfate) backbone started from commercially available material, GlaA-pnp. To introduce a GlcNTFA residue, GlcA-pnp (7 mM) was incubated with pmHS2 (150 μg/mL) in a buffer-containing Tris (25 mM, pH 7.5), MnCl₂ (15 mM) and UDP-GlcNTFA (10 mM), at 37° C. overnight. To introduce a GlcA residue, disaccharide substrate, GlcNTFA-GlcA-pnp (7 mM), was incubated with pmHS2 (100 μg/mL) in a buffer-containing Tris (25 mM, pH 7.5), MnCl2 (15 mM) and UDP-GlcA (1.5 mM), at 37° C. overnight. C₁₈ column (0.75×20 cm; Biotage, Uppsala, Sweden) was used for purification with gradient elution (0-100% methanol in H₂O, 0.10% trifluoroacetic acid, 2 mL/min in 60 min). The product was further elongated to 6mer under the same condition. Detrifluoroacetylation was completed using 0.1 M LiOH at 0° C. for 2 h. The solution was neutralized with chloric acid and N-sulfation was performed within pH 7.0 (3-(N-morpholino)-propanesulfonic acid) (MOPS) (50 mM), N-sulfotransferase (10 μg/mL) and PAPS (1.2 equiv. of each substrate amino group amount) at 37° C. overnight. The sulfated products were purified by Q-Sepharose column (15×200 mm, GE Health Sciences, Chicago, Illinois, United States of America) with linear gradient elution (20-100% 1 M NaCl in 20 mM NaOAc, pH 5.0, flow rate 1 mL/min). Afterwards, the NS 6mer was subject to epimerization and 2-O-sulfation within the solution contained MOPS (50 mM, pH 7.0), C₅-epi (80 μg/mL), 2-OST (80 μg/mL), PAPS (1.2 equiv. of substrate amount) at 37° C. overnight. Additional GlcA residue was introduced following the same procedure to give the compound NS2S 7mer. Following this elongation, a supplementary epimerization and 2-O-sulfation reaction was performed to obtain a second iduronic 2-O—S residue. 6-0 sulfation was then introduced using PAPS (1.2 equiv. of each substrate amino group amount), MOPS (50 mM, pH 7.0), and 6-OST-3 (0.7 mg/mL) overnight at 37° C. 3-O-Sulfation was finally performed under the condition of MOPS (50 mM, pH 7.0), 3-OST-1 (0.03 μg/mL) and PAPS (1.2 equiv. of substrate amount) at 37° C. overnight. Consequently, as shown in FIG. 7, the 7mer was incubated with UDP-GalNAc in a buffer containing 25 mM Tris (pH 7.5), 15 mM MnCl₂ and kfoC (170 μg/mL) overnight at 37° C. The backbone elongation continued using the same conditions by adding UDP-GlcA, UDP-GalNAc and again UDP-GlcA, hence the hybrid 11mer was obtained. The backbone was elongated introducing an additional GlcNTFA residue with pmHS2 (150 μg/mL) in a buffer-containing Tris (25 mM, pH 7.5), MnCl2 (15 mM) and UDP-GlcNTFA (10 mM), at 37° C. overnight. Detrifluoroacetylation on the last GlcNTFA residue was completed using 0.1 M LiOH at 0° C. for 2 h. pH was adjusted to 8.5, then, as shown in FIG. 7, 6 eq of succinimidyl 6-azidohexanate was added and the reaction mixture was left overnight at room temperature. The purification of the final product and of all intermediates were performed using Q-Sepharose column (15×200 mm, GE Health Sciences, Chicago, Illinois, United States of America) with linear gradient elution (0-100% 2 M NaCl in 20 mM NaOAc, pH 5.0, flow rate 1 mL/min).

Compound 1: As shown in FIG. 10, the elongation of the HS backbone started from commercially available material, GlaA-pnp. To introduce a GlcNTFA residue, GlcA-pnp (7 mM) was incubated with pmHS2 (150 μg/mL) in a buffer-containing Tris (25 mM, pH 7.5), MnCl₂ (15 mM) and UDP-GlcNTFA (10 mM), at 37° C. overnight. To introduce a GlcA residue, disaccharide substrate, GlcNTFA-GlcA-pnp (7 mM), was incubated with pmHS2 (100 μg/mL) in a buffer-containing Tris (25 mM, pH 7.5), MnCl₂ (15 mM) and UDP-GlcA (1.5 mM), at 37° C. overnight. C₁₈ column (0.75×20 cm; Biotage, Uppsala, Sweden) was used for purification with gradient elution (0-100% methanol in H2O, 0.1% trifluoroacetic acid, 2 mL/min in 60 min). The product was further elongated to 6mer under the same condition. Detrifluoroacetylation was completed using 0.1 M LiOH at 0° C. for 2 h. The solution was neutralized with chloric acid and N-sulfation was performed within pH 7.0 (3-(N-morpholino)-propanesulfonic acid) (MOPS) (50 mM), N-sulfotransferase (10 μg/mL) and PAPS (1.2 equiv. of each substrate amino group amount) at 37° C. overnight. The sulfated products were purified by Q-Sepharose column (15×200 mm, GE Health Sciences, Chicago, Illinois, United States of America) with linear gradient elution (20-100% 1 M NaCl in 20 mM NaOAc, pH 5.0, flow rate 1 mL/min). Afterwards, the NS 6mer was subject to epimerization and 2-O-sulfation within the solution contained MOPS (50 mM, pH 7.0), C₅-epi (80 μg/mL), 2-OST (80 μg/mL), PAPS (1.2 equiv. of substrate amount) at 37° C. overnight. Additional GlcA residue was introduced following the same procedure to give the compound NS2S 7mer. 6-0 sulfation was then introduced using PAPS (1.2 equiv. of each substrate amino group amount), MOPS (50 mM, pH 7.0), and 6-OST-3 (0.7 mg/mL) overnight at 37° C. 3-O-Sulfation was finally performed under the condition of MOPS (50 mM, pH 7.0), 3-OST-1 (0.03 μg/mL) and PAPS (1.2 equiv. of substrate amount) at 37° C. overnight. Consequently, 7mer was incubated with UDP-GlcNAc in a buffer containing 25 mM Tris (pH 7.5), 15 mM MnCl₂ and pmHS2 (150 μg/mL) overnight at 37° C. The backbone elongation continued using the same conditions by adding UDP-GlcA, UDP-GlcNAc and again UDP-GlcA until 11mer was obtained. The backbone was elongated introducing an additional GlcNTFA residue with pmHS2 (150 μg/mL) in a buffer-containing Tris (25 mM, pH 7.5), MnCl₂ (15 mM) and UDP-GlcNTFA (10 mM), at 37° C. overnight. Detrifluoroacetylation on the last GlcNTFA residue was completed using 0.1 M LiOH at 0° C. for 2 h. The pH was adjusted to 8.5, then 6 eq of succinimidyl 6-azidohexanate was added and the reaction mixture was left overnight at room temperature. The purification of the final product and of all intermediates were performed using Q-Sepharose column (15×200 mm, GE Health Sciences, Chicago, Illinois, United States of America) with linear gradient elution (0-100% 2 M NaCl in 20 mM NaOAc, pH 5.0, flow rate 1 mL/min).

Compound 4: As shown in FIG. 8, the elongation of the HS backbone started from commercially available material, GlaA-pnp. To introduce a GlcNTFA residue, GlcA-pnp (7 mM) was incubated with pmHS2 (150 μg/mL) in a buffer-containing Tris (25 mM, pH 7.5), MnCl₂ (15 mM) and UDP-GlcNTFA (10 mM), at 37° C. overnight. To introduce a GlcA residue, disaccharide substrate, GlcNTFA-GlcA-pnp (7 mM), was incubated with pmHS2 (100 μg/mL) in a buffer-containing Tris (25 mM, pH 7.5), MnCl₂ (15 mM) and UDP-GlcA (1.5 mM), at 37° C. overnight. C₁₈ column (0.75×20 cm; Biotage, Uppsala, Sweden) was used for purification with gradient elution (0-100% methanol in H₂O, 0.10% trifluoroacetic acid, 2 mL/min in 60 min). The product was further elongated to 6mer under the same condition. Detrifluoroacetylation was completed using 0.1 M LiOH at 0° C. for 2 h. The solution was neutralized with chloric acid and N-sulfation was performed within pH 7.0 (3-(N-morpholino)-propanesulfonic acid) (MOPS) (50 mM), N-sulfotransferase (10 μg/mL) and PAPS (1.2 equiv. of each substrate amino group amount) at 37° C. overnight. The sulfated products were purified by Q-Sepharose column (15×200 mm, GE Health Sciences, Chicago, Illinois, United States of America) with linear gradient elution (20-100% 1 M NaCl in 20 mM NaOAc, pH 5.0, flow rate 1 mL/min). Afterwards, the NS 6mer was subject to epimerization and 2-O-sulfation within the solution contained MOPS (50 mM, pH 7.0), C₅-epi (80 μg/mL), 2-OST (80 μg/mL), PAPS (1.2 equiv. of substrate amount) at 37° C. overnight. Additional GlcA residue was introduced following the same procedure to give the compound NS2S 7mer. Following this elongation, a supplementary epimerization and 2-O-sulfation reaction was performed to obtain a second iduronic 2-O—S residue. 6-0 sulfation was then introduced using PAPS (1.2 equiv. of each substrate amino group amount), MOPS (50 mM, pH 7.0), and 6-OST-3 (0.7 mg/mL) overnight at 37° C. Consequently, 7mer was incubated with UDP-GalNAc in a buffer containing 25 mM Tris (pH 7.5), 15 mM MnCl₂ and kfoC (170 μg/mL) overnight at 37° C. The backbone was further elongated using the same conditions by adding UDP-GlcA, hence the hybrid 9mer was obtained. To the oligo-pnp (1 mg/mL) 0.25 mg Pd/C was added. The reaction system was vacuumed and refilled with H₂ three times. After 4 h at room temperature the reaction was filtered, pH was adjusted to 8.5, then 6 eq of 6-heptynoic acid succinimidyl ester was added and the reaction mixture was left overnight at room temperature. The purification of the final product and of all intermediates were performed using Q-Sepharose column (15×200 mm, GE Health Sciences, Chicago, Illinois, United States of America) with linear gradient elution (0-100% 2 M NaCl in 20 mM NaOAc, pH 5.0, flow rate 1 mL/min).

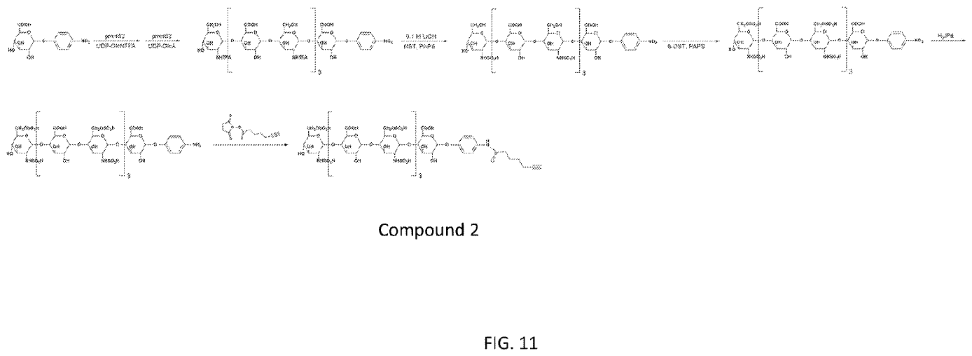

Compound 2: As shown in FIG. 11, the elongation of the HS backbone started from commercially available material, GlaA-pnp. To introduce a GlcNTFA residue, GlcA-pnp (7 mM) was incubated with pmHS2 (150 μg/mL) in a buffer-containing Tris (25 mM, pH 7.5), MnCl₂ (15 mM) and UDP-GlcNTFA (10 mM), at 37° C. overnight. To introduce a GlcA residue, disaccharide substrate, GlcNTFA-GlcA-pnp (7 mM), was incubated with pmHS2 (100 μg/mL) in a buffer-containing Tris (25 mM, pH 7.5), MnCl₂ (15 mM) and UDP-GlcA (1.5 mM), at 37° C. overnight. C₁₈ column (0.75×20 cm; Biotage, Uppsala, Sweden) was used for purification with gradient elution (0-100% methanol in H₂O, 0.10% trifluoroacetic acid, 2 mL/min in 60 min). The product was further elongated to 8mer under the same condition. Detrifluoroacetylation was completed using 0.1 M LiOH at 0° C. for 2 h. The solution was neutralized with chloric acid and N-sulfation was performed within pH 7.0 (3-(N-morpholino)-propanesulfonic acid) (MOPS) (50 mM), N-sulfotransferase (10 μg/mL) and PAPS (1.2 equiv. of each substrate amino group amount) at 37° C. overnight. The sulfated products were purified by Q-Sepharose column (15×200 mm, GE Health Sciences) with linear gradient elution (20-100% 1 M NaCl in 20 mM NaOAc, pH 5.0, flow rate 1 mL/min). 6-0 sulfation was then introduced using PAPS (1.2 equiv. of each substrate amino group amount), MOPS (50 mM, pH 7.0), and 6-OST-3 (0.7 mg/mL) overnight at 37° C. To oligo-pnp (1 mg/mL) was added 0.25 mg Pd/C. The reaction system was vacuumed and refilled with H₂ three times. After 4 h at room temperature the reaction was filtered, pH was adjusted to 8.5, then 6 eq of 6-heptynoic acid succinimidyl ester was added and the reaction mixture was left overnight at room temperature. The purification of the final product and of all intermediates were performed using Q-Sepharose column (15×200 mm, GE Health Sciences) with linear gradient elution (0-100% 2 M NaCl in 20 mM NaOAc, pH 5.0, flow rate 1 mL/min).

### Click Reactions:

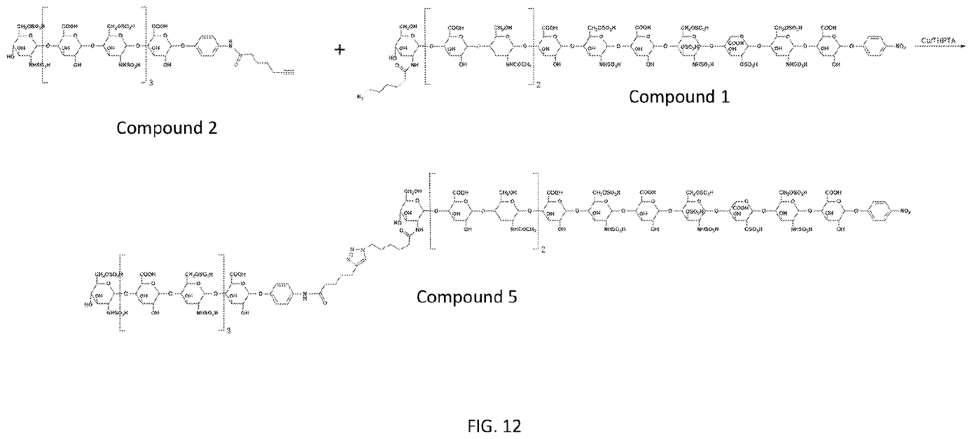

Compound 1 and Compound 2 were combined to form Compound 5. See FIG. 12. Compound 3 and Compound 4 were combined to form Compound 6. See FIG. 9. For the reactions, the reactants, i.e., the azide-functionalized and alkyne-functionalized compounds, were mixed together. PBS (pH 7.4) was bubbled with N₂ gas for 30 minutes to remove oxygen. Solutions of CuSO₄, (tris-hydroxypropyltriazolylmethylamine) (THPTA) and sodium ascorbate were prepared using bubbled PBS. CuSO₄ and THPTA were mixed to give a 1.5:5:1 ratio with the reactants. After vortex for one minute 6 equivalents (eq) of sodium ascorbate solution relative to Cu were added. The mixture of three reagents was pipetted into the reactants mixture within one minute after mixing all three reagents together. The reaction mixture was sealed with parafilm to prevent oxygen entry and left at 37° C. overnight. The purification of the final product was performed using Q-Sepharose column (15×200 mm, GE Health Sciences, Chicago, Illinois, United States of America) with linear gradient elution (0-100% 2 M NaCl in 20 mM NaOAc, pH 5.0, flow rate 1 mL/min).

### Anti-FIIa Activity

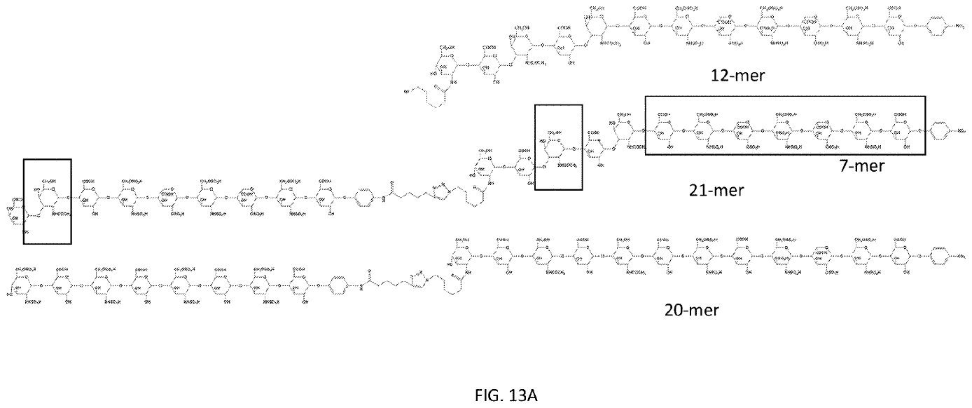

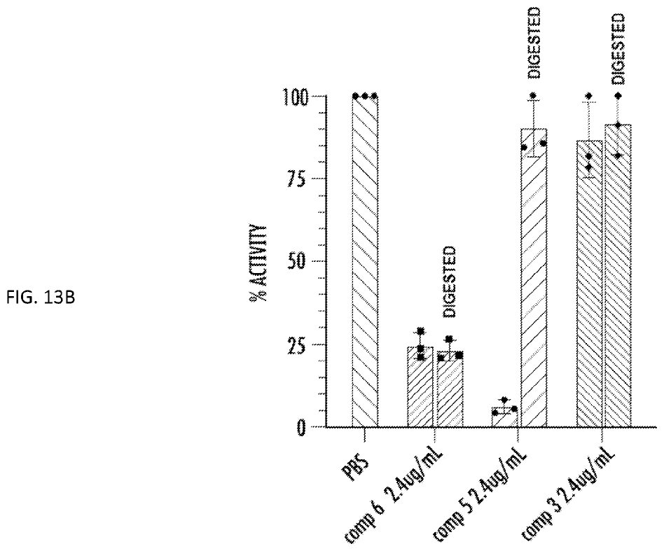

The anti-coagulant activities of Compound 5 and Compound 6 were evaluated by testing the inhibitory effect of the compounds when incubated with FIIa at a concentration of 2.4 μg/mL. As controls, PBS and Compound 3 were used. FIIa activity was measured both before and after heparanase digestion of the compounds. The structures of Compounds 3, 6, and 5 are shown for comparison in FIG. 13A. FIIa activity results are shown in FIG. 13B, where the results for each compound are shown as a pair of bars, with the bar on the left representing FIIa activity with the compound prior to digestion and the baron the right representing FIIa activity with the compound after digestion. The lower the activity, the greater the inhibitor effect. The inhibitory effect of Compound 6 was preserved after heparanase digestion.

### Analysis of Heparanase Digestion

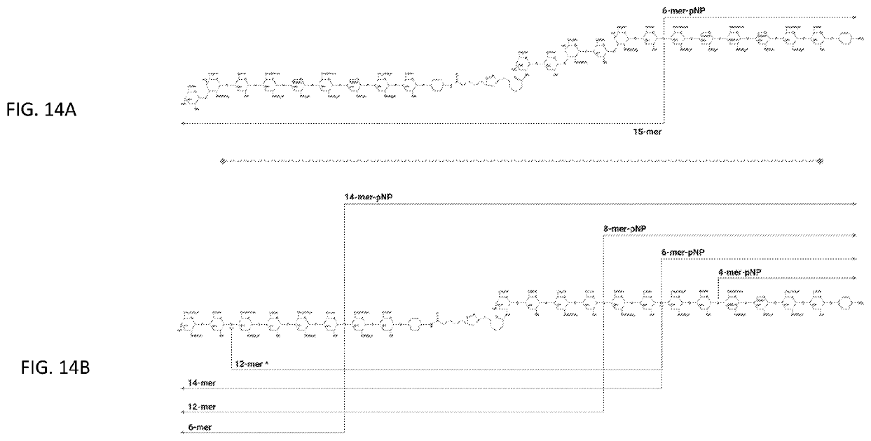

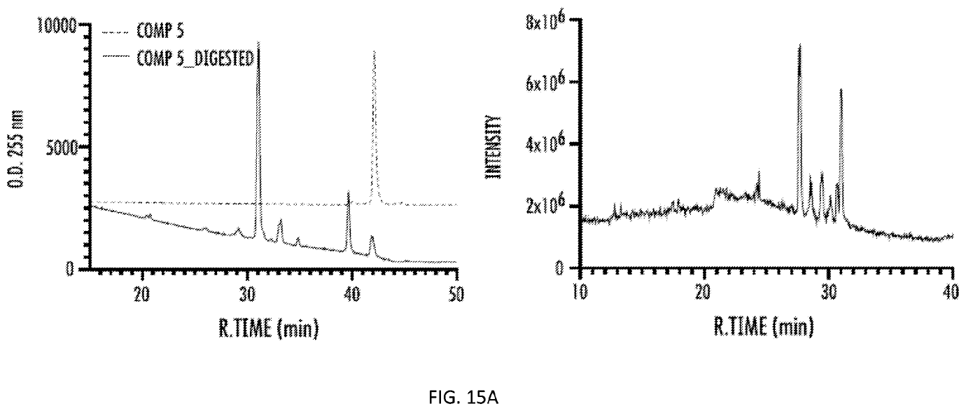

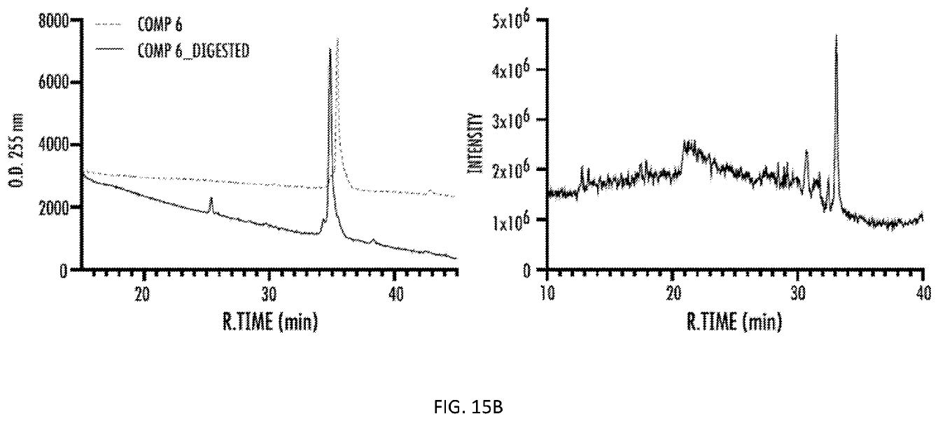

The heparanase digestion of Compound 6 and Compound 5 was analyzed by HPLC and MS. The HPLC chromatograms of Compound 5 before and after heparanase digestion are shown in FIG. 15A, while those of Compound 6 are shown in FIG. 15B. The digested oligosaccharide fragments from both compounds were identified by LC/MS. Table 2, below, summarizes the fragments from Compound 6 (with both calculated and measured molecular weights (MW)). Table 3, below summarizes the fragments from Compound 5 (with both calculated and measured molecular weights (MW)). The structural analysis of the fragmentation (indicating cleavage sites and detected digestion products) is shown in FIGS. 14A and 14B.

TABLE 2
Oligosaccharide Fragments of Compound
6 after Heparanase Digestion.

| Detected Product | Calculated MW | Measured MW |
|---|---|---|
| Compound 6 | 5531.45 | 5530.64 ± 0.2 |
| 6-mer-pNP | 1870.47 | 1871.58 ± 0.3 |
| 15-mer | 3677.10 | 3676.71 ± 0.3 |

TABLE 3
Oligosaccharide Fragments of Compound
6 after Heparanase Digestion.

| Detected product | Calculated MW | Measured MW |
|---|---|---|
| Compound 5 | 5232.33 | 5231.85 ± 0.2 |
| 14-mer-pNP | 3740.15 | 3740.07 ± 0.2 |
| 8-mer-pNP | 2169.73 | 2169.72 ± 0.4 |
| 6-mer-pNP | 1790.41 | 1790.46 ± 0.3 |
| 4-mer-pNP | 1293.01 | 1293.68 ± 0.3 |
| 12-mer * | 2959.53 | 2959.98 ± 0.2 |
| 14-mer | 3458.94 | 3461.01 ± 0.2 |
| 12-mer | 3079.62 | 3079.41 ± 0.3 |
| 6-mer | 1509.20 | 1509 ± 0.3 |

### REFERENCES

1. Klein U, Kresse H, von Figura K. Evidence for degradation of heparan sulfate by endoglycosidases: Glucosamine and hexuronic acid are reducing terminals of intracellular heparan sulfate from human skin fibroblasts. Biochem Biophys Res Commun 1976; 69(1):158-66.
2. Sivaram P, Obunike J C, Goldberg I J. Lysolecithin-induced alteration of subendothelial heparan sulfate proteoglycans increases monocyte binding to matrix. J Biol Chem 1995; 270(50):29760-5.
3. Oldberg A, Heldin C H, Wasteson A, Busch C, Hook Mₙ Characterization of a platelet endoglycosidase degrading heparin-like polysaccharides. Biochemistry 1980; 19(25):5755-62.
4. Matzner Y, Bar-Ner M, Yahalom J, Ishai-Michaeli R, Fuks Z, Vlodavsky I. Degradation of heparan sulfate in the subendothelial extracellular matrix by a readily released heparanase from human neutrophils. Possible role in invasion through basement membranes. J Cin Invest 1985; 76(4):1306-13.
5. Naparstek Y, Cohen I R, Fuks Z, Vlodavsky I. Activated T lymphocytes produce a matrix-degrading heparan sulphate endoglycosidase. Nature 1984; 310(5974):241-4.
6. Hulett M D, Freeman C, Hamdorf B J, Baker R T, Harris M J, Parish C R. Cloning of mammalian heparanase, an important enzyme in tumor invasion and metastasis. Nat Med 1999; 5(7):803-9.
7. Gong F, Jemth P, Escobar Galvis M L, Vlodavsky I, Horner A, Lindahl U, Li J P. Processing of macromolecular heparin by heparanase. J Biol Chem 2003; 278(37):35152-8.
8. Nakajima M, Irimura T, Di Ferrante D, Di Ferrante N, Nicolson G L. Heparan sulfate degradation: Relation to tumor invasive and metastatic properties of mouse B16 melanoma sublines. Science 1983; 220(4597):611-3.
9. Vlodavsky I, Friedmann Y. Molecular properties and involvement of heparanase in cancer metastasis and angiogenesis. J Cin Invest 2001; 108(3):341-7.
10. Pikas D S, Li J P, Vlodavsky I, Lindahl U. Substrate specificity of heparanases from human hepatoma and platelets. J Biol Chem 1998; 273(30):18770-7.
11. Wu L, Viola C M, Brzozowski A M, Davies G J. Structural characterization of human heparanase reveals insights into substrate recognition. Nat Struct Mol Biol 2015; 22(12):1016-22.
12. Thunberg L, Backstrom G, Wasteson A, Robinson H C, Ogren S, Lindahl U. Enzymatic depolymerization of heparin-related polysaccharides. Substrate specificities of mouse mastocytoma and human platelet endo-beta-D-glucuronidases. J Biol Chem 1982; 257(17):10278-82.
13. Nadir Y, Brenner B, Zetser A, Ilan N, Shafat I, Zcharia E, Goldshmidt O, Vlodavsky I. Heparanase induces tissue factor expression in vascular endothelial and cancer cells. J Thromb Haemost 2006; 4(11):2443-51.
14. Nadir Y, Brenner B, Gingis-Velitski S, Levy-Adam F, Ilan N, Zcharia E, Nadir E, Vlodavsky I. Heparanase induces tissue factor pathway inhibitor expression and extracellular accumulation in endothelial and tumor cells. Thromb Haemost 2008; 99(1):133-41.
15. Nadir Y, Brenner B, Fux L, Shafat I, Attias J, Vlodavsky I. Heparanase enhances the generation of activated factor X in the presence of tissue factor and activated factor VII. Haematologica 2010; 95(11):1927-34.
16. Nadir Y, Brenner B. Heparanase procoagulant activity in cancer progression. Thromb Res 2016; 140 Suppl 1:S44-8.
17. Baker A B, Gibson W J, Kolachalama V B, Golomb M, Indolfi L, Spruell C, Zcharia E, Vlodavsky I, Edelman E R. Heparanase regulates thrombosis in vascular injury and stent-induced flow disturbance. J Am Coll Cardiol 2012; 59(17):1551-60.
18. Waterman M, Ben-Izhak O, Eliakim R, Groisman G, Vlodavsky I, Ilan N. Heparanase upregulation by colonic epithelium in inflammatory bowel disease. Mod Pathol 2007; 20(1):8-14.
19. Li R W, Freeman C, Yu D, Hindmarsh E J, Tymms K E, Parish C R, Smith P N. Dramatic regulation of heparanase activity and angiogenesis gene expression in synovium from patients with rheumatoid arthritis. Arthritis Rheum 2008; 58(6):1590-600.
20. Osterholm C, Folkersen L, Lengquist M, Ponten F, Renne T, Li J, Hedin U. Increased expression of heparanase in symptomatic carotid atherosclerosis. Atherosclerosis 2013; 226(1):67-73.
21. Shafat I, Agbaria A, Boaz M, Schwartz D, Baruch R, Nakash R, Ilan N, Vlodavsky I, Weinstein T. Elevated urine heparanase levels are associated with proteinuria and decreased renal allograft function. PLoS One 2012; 7(9):e44076.
22. Shafat I, Ilan N, Zoabi S, Vlodavsky I, Nakhoul F. Heparanase levels are elevated in the urine and plasma of type 2 diabetes patients and associate with blood glucose levels. PLoS One 2011; 6(2):e17312.
23. Peled E, Rovitsky A, Axelman E, Norman D, Brenner B, Nadir Y. Increased heparanase level and procoagulant activity in orthopedic surgery patients receiving prophylactic dose of enoxaparin. Thromb Res 2012; 130(1):129-34.
24. Matan M, Axelman E, Brenner B, Nadir Y. Heparanase procoagulant activity is elevated in women using oral contraceptives. Hum Reprod 2013; 28(9):2372-80.
25. Bayam E, Kalcik M, Gurbuz A S, Yesin M, Guner A, Gunduz S, Gursoy M O, Karakoyun S, Cersit S, Kilicgedik A, Candan O, Yaman A, Ozkan M. The relationship between heparanase levels, thrombus burden and thromboembolism in patients receiving unfractionated heparin treatment for prosthetic valve thrombosis. Thromb Res 2018; 171:103-10.
26. Friedmann Y, Vlodavsky I, Aingorn H, Aviv A, Peretz T, Pecker I, Pappo O. Expression of heparanase in normal, dysplastic, and neoplastic human colonic mucosa and stroma. Evidence for its role in colonic tumorigenesis. Am J Pathol 2000; 157(4):1167-75.
27. Koliopanos A, Friess H, Kleeff J, Shi X, Liao Q, Pecker I, Vlodavsky I, Zimmermann A, Buchler M W. Heparanase expression in primary and metastatic pancreatic cancer. Cancer Res 2001; 61(12):4655-9.
28. Tang W, Nakamura Y, Tsujimoto M, Sato M, Wang X, Kurozumi K, Nakahara M, Nakao K, Nakamura M, Mori I, Kakudo K. Heparanase: A key enzyme in invasion and metastasis of gastric carcinoma. Mod Pathol 2002; 15(6):593-8.
29. Quiros R M, Rao G, Plate J, Harris J E, Brunn G J, Platt J L, Gattuso P, Prinz R A, Xu X. Elevated serum heparanase-1 levels in patients with pancreatic carcinoma are associated with poor survival. Cancer 2006; 106(3):532-40.
30. Nadir Y, Sang G, Axelman E, Meir A, Wollner M, Shafat I, Hoffman R, Brenner B, Vlodavsky I, Haim N. Heparanase procoagulant activity is elevated and predicts survival in non-small cell lung cancer patients. Thromb Res 2014; 134(3):639-42.
31. Maurice-Dror C, Litvak M, Keren-Politansky A, Ackerman S, Haim N, Nadir Y. Circulating heparan sulfate chains and body weight contribute to anti-Xa levels in cancer patients using the prophylactic dose of enoxaparin. J Thromb Thrombolysis 2020; 50(1):112-22.
32. Freeman C, Parish C R. Human platelet heparanase: Purification, characterization and catalytic activity. Biochem J 1998; 330 (Pt 3):1341-50.
33. Nasser N J, Sarig G, Brenner B, Nevo E, Goldshmidt O, Zcharia E, Li J P, Vlodavsky I. Heparanase neutralizes the anticoagulation properties of heparin and low-molecular-weight heparin. J Thromb Haemost 2006; 4(3):560-5.
34. Carrier M, Abou-Nassar K, Mallick R, Tagalakis V, Shivakumar S, Schattner A, Kuruvilla P, Hill D, Spadafora S, Marquis K, Trinkaus M, Tomiak A, Lee A Y Y, Gross P L, Lazo-Langner A, El-Maraghi R, Goss G, Le Gal G, Stewart D, Ramsay T, Rodger M, Witham D, Wells P S. Apixaban to prevent venous thromboembolism in patients with cancer. N Engl J Med 2019; 380(8):711-9.
35. Khorana A A, Soff G A, Kakkar A K, Vadhan-Raj S, Riess H, Wun T, Streiff M B, Garcia D^{A}, Liebman H A, Belani C P, O'Reilly E M, Patel J N, Yimer H A, Wildgoose P, Burton P, Vijapurkar U, Kaul S, Eikelboom J, McBane R, Bauer K A, Kuderer N M, Lyman G H, Investigators C. Rivaroxaban for thromboprophylaxis in high-risk ambulatory patients with cancer. N Engl J Med 2019; 380(8):720-8.
36. Young A M, Marshall A, Thirlwall J, Chapman O, Lokare A, Hill C, Hale D, Dunn J A, Lyman G H, Hutchinson C, MacCallum P, Kakkar A, Hobbs F D R, Petrou S, Dale J, Poole C J, Maraveyas A, Levine Mₙ Comparison of an oral factor Xa inhibitor with low molecular weight heparin in patients with cancer with venous thromboembolism: Results of a randomized trial (SELECT-D). J Clin Oncol 2018; 36(20):2017-23.
37. Xu Y, Masuko S, Takieddin M, Xu H, Liu R, Jing J, Mousa S A, Linhardt R J, Liu J. Chemoenzymatic synthesis of homogeneous ultralow molecular weight heparins. Science 2011; 334(6055):498-501.
38. Xu Y, Pempe E H, Liu J. Chemoenzymatic synthesis of heparin oligosaccharides with both anti-factor Xa and anti-factor IIa activities. J Biol Chem 2012; 287(34):29054-61.
39. Peterson S B, Liu J. Unraveling the specificity of heparanase utilizing synthetic substrates. J Biol Chem 2010; 285(19):14504-13.
40. Peterson S, Liu J. Deciphering mode of action of heparanase using structurally defined oligosaccharides. J Biol Chem 2012; 287(41):34836-43.
41. Peterson S B, Liu J. Multi-faceted substrate specificity of heparanase. Matrix Biol 2013; 32(5):223-7.
42. Liu J, Shworak N W, Fritze L M, Edelberg J M, Rosenberg R D. Purification of heparan sulfate D-glucosaminyl 3-O-sulfotransferase. J Biol Chem 1996; 271(43):27072-82.
43. Xu Y, Cai C, Chandarajoti K, Hsieh P H, Li L, Pham T Q, Sparkenbaugh E M, Sheng J, Key N S, Pawlinski R, Harris E N, Linhardt R J, Liu J. Homogeneous low-molecular-weight heparins with reversible anticoagulant activity. Nat Chem Biol 2014; 10(4):248-50.
44. Wang Z, Hsieh P H, Xu Y, Thieker D, Chai E J E, Xie S, Cooley B, Woods R J, Chi L, Liu J. Synthesis of 3-O-sulfated oligosaccharides to understand the relationship between structures and functions of heparan sulfate. J Am Chem Soc 2017; 139(14):5249-56.
45. Xu Y, Chandarajoti K, Zhang X, Pagadala V, Dou W, Hoppensteadt D M, Sparkenbaugh E M, Cooley B, Daily S, Key N S, Severynse-Stevens D, Fareed J, Linhardt R J, Pawlinski R, Liu J. Synthetic oligosaccharides can replace animal-sourced low-molecular weight heparins. Sci Transl Med 2017; 9(406).
46. Hanjaya-Putra D, Haller C, Wang X, Dai E, Lim B, Liu L, Jaminet P, Yao J, Searle A, Bonnard T, Hagemeyer C E, Peter K, Chaikof E L. Platelet-targeted dual pathway antithrombotic inhibits thrombosis with preserved hemostasis. JCI Insight 2018; 3(15).
47. Linhardt R J, Liu J. Synthetic heparin. Curr Opin Pharmacol 2012; 12(2):217-9.
48. Cai C, Li L, Harvey C, Liu J, Linhardt R J. Towards the chemoenzymatic synthesis of heparan sulfate oligosaccharides: Oxidative cleavage of p-nitrophenyl group with ceric ammonium salts. Tetrahedron Lett 2013; 54(33):4471-4.
49. Chen M, Bridges A, Liu J. Determination of the substrate specificities of N-acetyl-d-glucosaminyltransferase. Biochemistry 2006; 45(40):12358-65.
50. Liu R, Xu Y, Chen M, Weiwer M, Zhou X, Bridges A S, DeAngelis P L, Zhang Q, Linhardt R J, Liu J. Chemoenzymatic design of heparan sulfate oligosaccharides. J Biol Chem 2010; 285(44):34240-9.
51. McRae S J, Ginsberg J S. Initial treatment of venous thromboembolism. Circulation 2004; 110(9 Suppl 1):13-9.
52. Khorana A A, Francis C W, Culakova E, Kuderer N M, Lyman G H. Thromboembolism is a leading cause of death in cancer patients receiving outpatient chemotherapy. J Thromb Haemost 2007; 5(3):632-4.
53. Walker A J, Card T R, West J, Crooks C, Grainge M J. Incidence of venous thromboembolism in patients with cancer—a cohort study using linked United Kingdom databases. Eur J Cancer 2013; 49(6):1404-13.
54. Prandoni P, Lensing A W, Piccioli A, Bernardi E, Simioni P, Girolami B, Marchiori A, Sabbion P, Prins M H, Noventa F, Girolami A. Recurrent venous thromboembolism and bleeding complications during anticoagulant treatment in patients with cancer and venous thrombosis. Blood 2002; 100(10):3484-8.
55. Lyman G H, Khorana A A, Kuderer N M, Lee A Y, Arcelus J I, Balaban E P, Clarke J M, Flowers C R, Francis C W, Gates L E, Kakkar A K, Key N S, Levine M N, Liebman H A, Tempero M A, Wong S L, Prestrud A A, Falanga A, American Society of Clinical Oncology Clinical P. Venous thromboembolism prophylaxis and treatment in patients with cancer: American Society of Clinical Oncology clinical practice guideline update. J Clin Oncol 2013; 31(17):2189-204.
56. Devine C, McCollum C, North West Femoro-Popliteal Trial P. Heparin-bonded Dacron or polytetrafluorethylene for femoropopliteal bypass: Five-year results of a prospective randomized multicenter clinical trial. J Vasc Surg 2004; 40(5):924-31.
57. Lindholt J S, Gottschalksen B, Johannesen N, Dueholm D, Ravn H, Christensen E D, Viddal B, Florenes T, Pedersen G, Rasmussen M, Carstensen M, Grondal N, Fasting H. The Scandinavian Propaten trial. 1-year patency of PTFE vascular prostheses with heparin-bonded luminal surfaces compared to ordinary pure PTFE vascular prostheses. A randomised clinical controlled multi-center trial. Eur J Vasc Endovasc Surg 2011; 41(5):668-73.
58. Lumsden A B, Morrissey N J. Randomized controlled trial comparing the safety and efficacy between the FUSION BIOLINE heparin-coated vascular graft and the standard expanded polytetrafluoroethylene graft for femoropopliteal bypass. J Vasc Surg 2015; 61(3):703-12.
59. Lensvelt M M, Fritschy W M, van Oostayen J A, Holewijn S, Zeebregts C J, Reijnen M M. Results of heparin-bonded ePTFE-covered stents for chronic occlusive superficial femoral artery disease. J Vasc Surg 2012; 56(1):118-25.
60. Ohki T, Kichikawa K, Yokoi H, Uematsu M, Yamaoka T, Maeda K, Kanaoka Y. Outcomes of the Japanese multicenter Viabahn trial of endovascular stent grafting for superficial femoral artery lesions. J Vasc Surg 2017; 66(1):130-42 el.
61. Mahmood S, Bilal H, Zaman M, Tang A. Is a fully heparin-bonded cardiopulmonary bypass circuit superior to a standard cardiopulmonary bypass circuit? Interact Cardiovasc Thorac Surg 2012; 14(4):406-14.
62. Fraser C D, Jr., Jaquiss R D, Rosenthal D N, Humpl T, Canter C E, Blackstone E H, Naftel D C, Ichord R N, Bomgaars L, Tweddell J S, Massicotte M P, Turrentine M W, Cohen G A, Devaney E J, Pearce F B, Carberry K E, Kroslowitz R, Almond C S, Berlin Heart Study I. Prospective trial of a pediatric ventricular assist device. N Engl J Med 2012; 367(6):532-41.
63. Naito N, Cook K, Toyoda Y, Shigemura N. Artificial lungs for lung failure. J Am Coll Cardiol 2018; 72(14):1640-52.
64. Devine C, Hons B, McCollum C. Heparin-bonded Dacron or polytetrafluoroethylene for femoropopliteal bypass grafting: a multicenter trial. J Vasc Surg 2001; 33(3):533-9.
65. Pedersen G, Laxdal E, Ellensen V, Jonung T, Mattsson E. Improved patency and reduced intimal hyperplasia in PTFE grafts with luminal immobilized heparin compared with standard PTFE grafts at six months in a sheep model. J Cardiovasc Surg (Torino) 2010; 51(3):443-8.
66. Shemesh D, Goldin I, Hijazi J, Zaghal I, Berelowitz D, Verstandig A, Olsha O. A prospective randomized study of heparin-bonded graft (Propaten) versus standard graft in prosthetic arteriovenous access. J Vasc Surg 2015; 62(1):115-22.
67. Lindholt J S, Houlind K, Gottschalksen B, Pedersen C N, Ravn H, Viddal B, Pedersen G, Rasmussen M, Wedel C, Bramsen M B. Five-year outcomes following a randomized trial of femorofemoral and femoropopliteal bypass grafting with heparin-bonded or standard polytetrafluoroethylene grafts. Br J Surg 2016; 103(10):1300-5.
68. Begovac P C, Thomson R^{C}, Fisher J L, Hughson A, Gallhagen A. Improvements in GORE-TEX vascular graft performance by Carmeda BioActive surface heparin immobilization. Eur J Vasc Endovasc Surg 2003; 25(5):432-7.
69. Freeman J, Chen A, Weinberg R J, Okada T, Chen C, Lin P H. Sustained thromboresistant bioactivity with reduced intimal hyperplasia of heparin-bonded polytetrafluoroethylene propaten graft in a chronic canine femoral artery bypass model. Ann Vasc Surg 2018; 49:295-303.
70. Peterson, S., Liu, J. Unraveling the specificity of heparanase utilizing synthetic substrates J. Biol. Chem. 2010; 285:14504-14513.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A heparin oligomer having a structure of Formula (I): 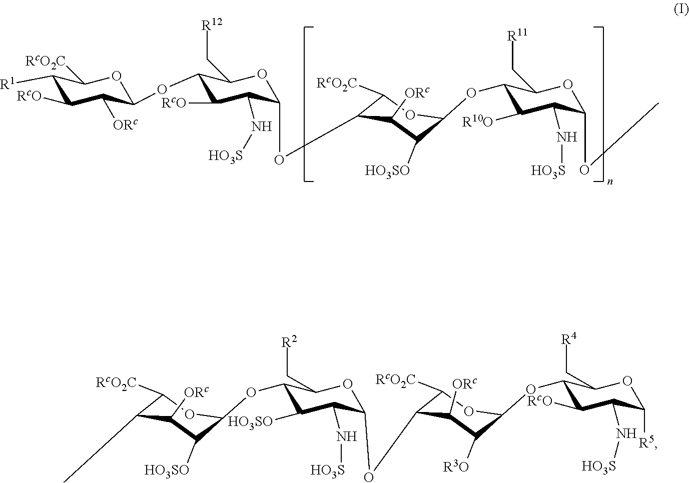
or a pharmaceutically acceptable salt, solvate, hydrate, polymorph, co-crystal, tautomer, stereoisomer, isotopically labeled derivative, or prodrug thereof, wherein:
n is 0, 1, 2, 3, 4, 5, 6, 7, 8, 9, or 10;
R¹ is —OR^{A}, —SR^{A}, —N(R^{A})₂, halogen, an optionally substituted monosaccharide, or an optionally substituted oligosaccharide;
each occurrence of R^{A} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atoms to form an optionally substituted heterocyclic ring or optionally substituted heteroaryl ring;
each of R², R⁴, R¹¹, and R¹² is independently —H, —OH, —OSO₃H, or —SO₃H;
each of R³ and R¹⁰ is independently —SO₃H, —H, optionally substituted C₁-C₆ alkyl, or an oxygen protecting group;
R⁵ is —OR^{B}, —SR^{B}, —N(R^{B})₂, halogen, an optionally substituted monosaccharide, or an optionally substituted oligosaccharide;
each occurrence of R^{B} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{B} are joined together with their intervening atoms to form an optionally substituted heterocyclic ring or optionally substituted heteroaryl ring; and
each occurrence of R^{C} is independently —H, optionally substituted C₁-C₆ alkyl, or an oxygen protecting group;
provided that one of R¹ and R⁵ is an optionally substituted monosaccharide.

2. The heparin oligomer of claim 1, wherein R¹¹ is —H, —OH, or —OSO₃H or wherein R¹⁰ is —H or —SO₃H or wherein n is 0, 1, or 2.

3. (canceled)

4. (canceled)

5. The heparin oligomer of claim 1, wherein the heparin oligomer has a structure of Formula (I-A): 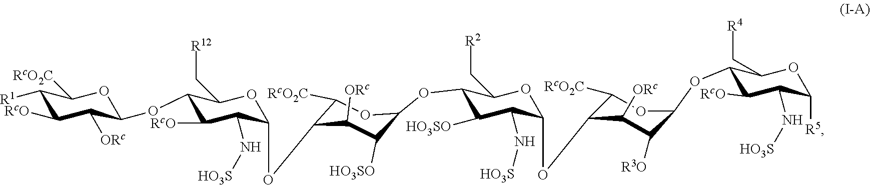
or a pharmaceutically acceptable salt, solvate, hydrate, polymorph, co-crystal, tautomer, stereoisomer, isotopically labeled derivative, or prodrug thereof, wherein:
R¹ is —OR^{A}, —SR^{A}, —N(R^{A})₂, halogen, an optionally substituted monosaccharide, or an optionally substituted oligosaccharide;
each occurrence of R^{A} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atoms to form an optionally substituted heterocyclic ring or optionally substituted heteroaryl ring;
each of R², R⁴, and R¹² is independently —H, —OH, —OSO₃H, or —SO₃H;
R³ is —H, —SO₃H, optionally substituted C₁-C₆ alkyl, or an oxygen protecting group;
R⁵ is —OR^{B}, —SR^{B}, —N(R^{B})₂, halogen, or an optionally substituted monosaccharide;
each occurrence of R^{B} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{B} are joined together with their intervening atoms to form an optionally substituted heterocyclic ring or optionally substituted heteroaryl ring; and
each occurrence of R^{C} is independently —H, optionally substituted C₁-C₆ alkyl, or an oxygen protecting group;
provided that one of R¹ and R⁵ is an optionally substituted monosaccharide.

6. The heparin oligomer of claim 1, or a pharmaceutically acceptable salt, solvate, hydrate, polymorph, co-crystal, tautomer, stereoisomer, isotopically labeled derivative, or prodrug thereof, wherein R² is —H, —OH, or —OSO₃H.

7. (canceled)

8. The heparin oligomer of claim 1, or a pharmaceutically acceptable salt, solvate, hydrate, polymorph, co-crystal, tautomer, stereoisomer, isotopically labeled derivative, or prodrug thereof, wherein R³ is —H or —SO₃H.

9. (canceled)

10. The heparin oligomer of claim 1, or a pharmaceutically acceptable salt, solvate, hydrate, polymorph, co-crystal, tautomer, stereoisomer, isotopically labeled derivative, or prodrug thereof, wherein R⁴ is —H, —OH, or —OSO₃H.

11. (canceled)

12. The heparin oligomer of claim 1, or a pharmaceutically acceptable salt, solvate, hydrate, polymorph, co-crystal, tautomer, stereoisomer, isotopically labeled derivative, or prodrug thereof, wherein R¹² is —H, —OH, or —OSO₃H.

13. (canceled)

14. The heparin oligomer of claim 5, or a pharmaceutically acceptable salt, solvate, hydrate, polymorph, co-crystal, tautomer, stereoisomer, isotopically labeled derivative, or prodrug thereof, wherein the heparin oligomer has a structure of a formula selected from the group consisting of: 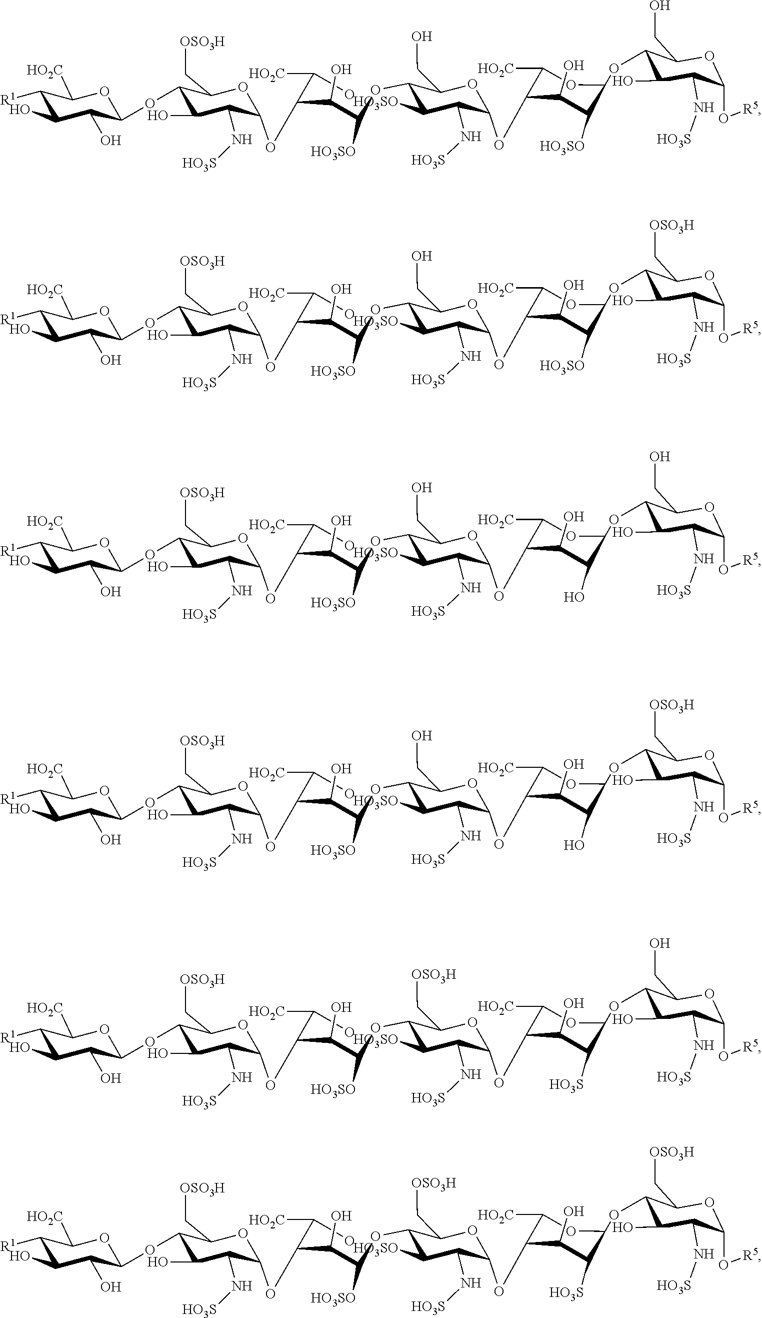 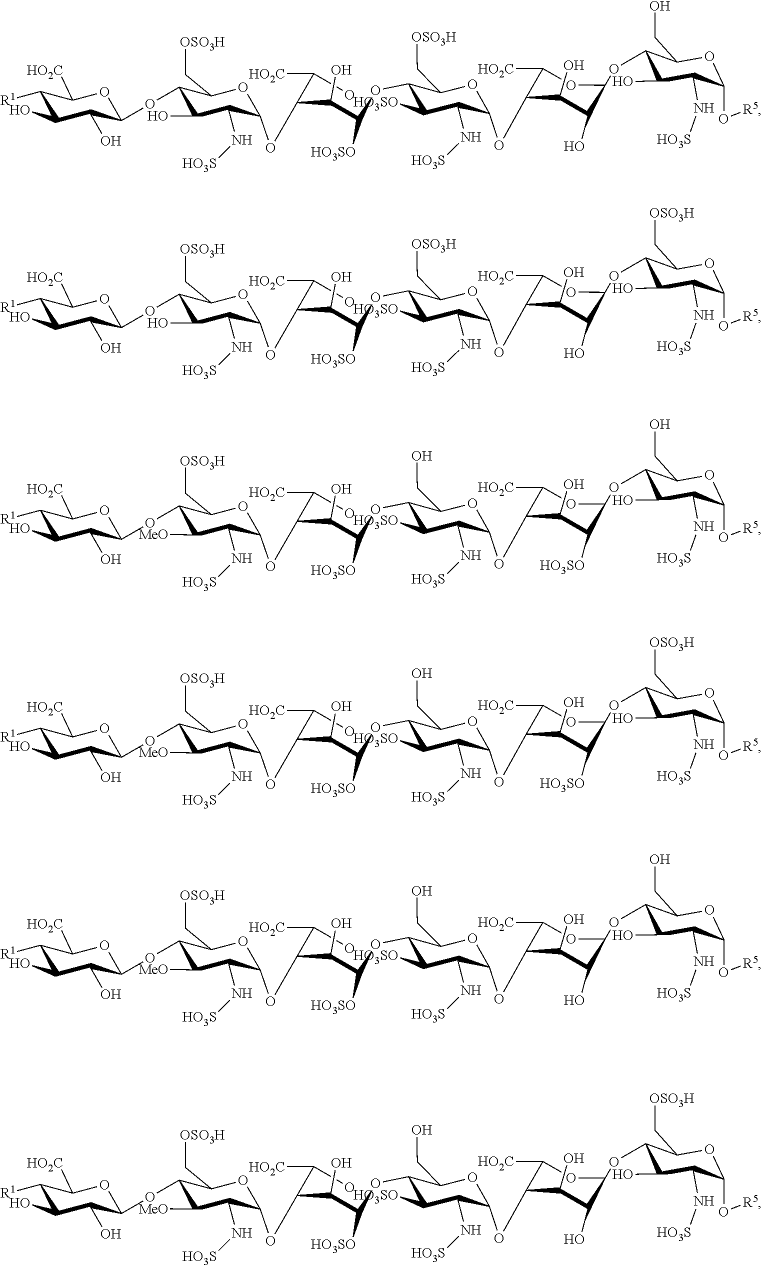 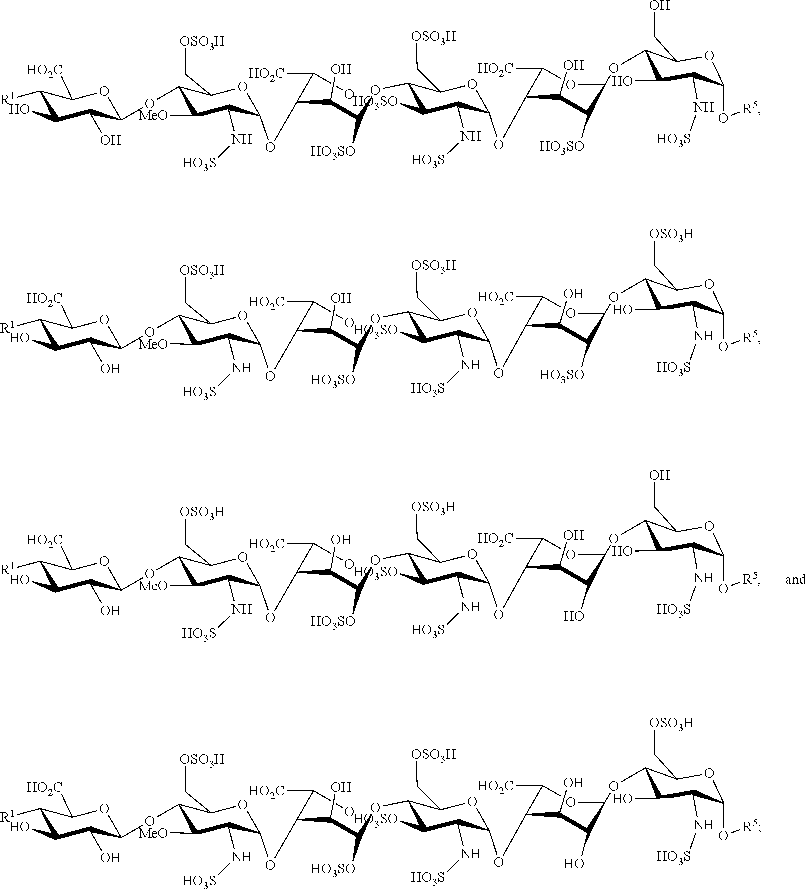
or a pharmaceutically acceptable salt, solvate, hydrate, polymorph, co-crystal, tautomer, stereoisomer, isotopically labeled derivative, or prodrug thereof.

15. The heparin oligomer of claim 14, or a pharmaceutically acceptable salt, solvate, hydrate, polymorph, co-crystal, tautomer, stereoisomer, isotopically labeled derivative, or prodrug thereof, wherein the heparin oligomer has a structure of the formula:

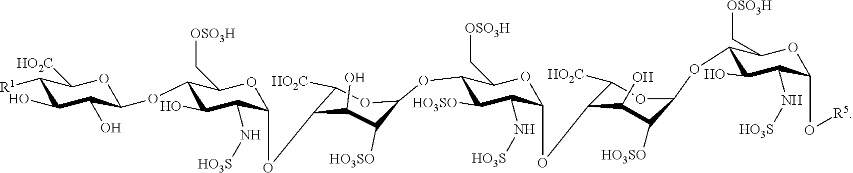

16. The heparin oligomer of claim 1, or a pharmaceutically acceptable salt, solvate, hydrate, polymorph, co-crystal, tautomer, stereoisomer, isotopically labeled derivative, or prodrug thereof, wherein R¹ is —OR^{A}, —SR^{A}, —N(R^{A})₂, or halogen.

17. The heparin oligomer of claim 1, or a pharmaceutically acceptable salt, solvate, hydrate, polymorph, co-crystal, tautomer, stereoisomer, isotopically labeled derivative, or prodrug thereof, wherein R^{A} is —H, a nitrogen protecting group when attached to a nitrogen atom, an oxygen protecting group when attached to an oxygen atom, or a sulfur protecting group when attached to a sulfur atom.

18. The heparin oligomer of claim 1, or a pharmaceutically acceptable salt, solvate, hydrate, polymorph, co-crystal, tautomer, stereoisomer, isotopically labeled derivative, or prodrug thereof, wherein R¹ is an optionally substituted monosaccharide.

19. The heparin oligomer of claim 18, or a pharmaceutically acceptable salt, solvate, hydrate, polymorph, co-crystal, tautomer, stereoisomer, isotopically labeled derivative, or prodrug thereof, wherein R¹ is optionally substituted glucosamine.

20. The heparin oligomer of claim 19, or a pharmaceutically acceptable salt, solvate, hydrate, polymorph, co-crystal, tautomer, stereoisomer, isotopically labeled derivative, or prodrug thereof, wherein R¹ is 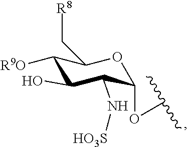
wherein:
R⁸ is —H, —OH, —OSO₃H, or —SO₃H; and
R⁹ is —H, an oxygen protecting group, optionally substituted aliphatic, optionally substituted heteroaliphatic, optionally substituted carbocyclyl, optionally substituted heterocyclyl, optionally substituted aryl, or optionally substituted heteroaryl.

21. The heparin oligomer of claim 20, or a pharmaceutically acceptable salt, solvate, hydrate, polymorph, co-crystal, tautomer, stereoisomer, isotopically labeled derivative, or prodrug thereof, wherein R⁸ is —H, —OH, or —OSO₃H.

22. The heparin oligomer of claim 20, or a pharmaceutically acceptable salt, solvate, hydrate, polymorph, co-crystal, tautomer, stereoisomer, isotopically labeled derivative, or prodrug thereof, wherein R⁹ is —H or an oxygen protecting group.

23. The heparin oligomer of claim 1, or a pharmaceutically acceptable salt, solvate, hydrate, polymorph, co-crystal, tautomer, stereoisomer, isotopically labeled derivative, or prodrug thereof, wherein R¹ is an optionally substituted oligosaccharide.

24. The heparin oligomer of claim 23, wherein the optionally substituted oligosaccharide comprises a pentasaccharide moiety with the structure: 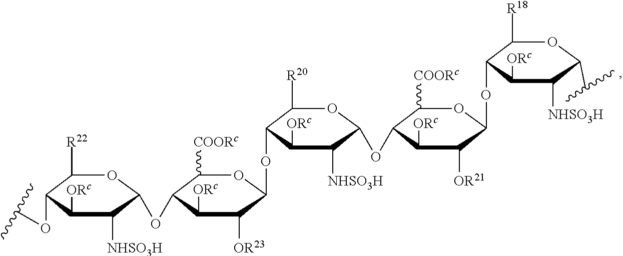
wherein:
each of R¹⁸, R²⁰, and R²² is independently —H, —OH, —OSO₃H, or —SO₃H;
each of R²¹ and R²³ is independently —SO₃H, —H, optionally substituted C₁-C₆ alkyl, or an oxygen protecting group; and
each occurrence of R^{C} is independently —H, optionally substituted C₁-C₆ alkyl, or an oxygen protecting group.

25. The heparin oligomer of claim 1, or a pharmaceutically acceptable salt, solvate, hydrate, polymorph, co-crystal, tautomer, stereoisomer, isotopically labeled derivative, or prodrug thereof, wherein R¹ is an optionally substituted monosaccharide or an optionally substituted oligosaccharide having a structure of the formula: 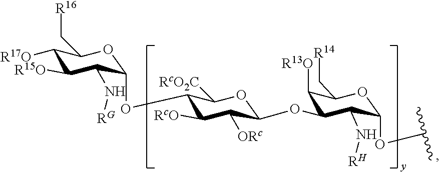
wherein:
y is 0, 1, 2, 3, 4, 5, or 6;
each of R¹³, R¹⁵, and R¹⁷ is independently —SO₃H, —H, optionally substituted C₁-C₆ alkyl, or an oxygen protecting group; and
each R¹⁴ is independently —OH, an oxygen protecting group, optionally substituted C₁-C₆ alkyl, or —OSO₃H;
R¹⁶ is —OH, an oxygen protecting group, optionally substituted C₁-C₆ alkyl, or —OSO₃H;
each occurrence of R^{C} is independently —H, optionally substituted C₁-C₆ alkyl, or an oxygen protecting group;
R^{H} is optionally substituted acyl; and
R^{G} is optionally substituted acyl or optionally substituted alkyl.

26. The heparin oligomer of claim 25, or a pharmaceutically acceptable salt, solvate, hydrate, polymorph, co-crystal, tautomer, stereoisomer, isotopically labeled derivative, or prodrug thereof wherein y is 2.

27. The heparin oligomer of claim 25, or a pharmaceutically acceptable salt, solvate, hydrate, polymorph, co-crystal, tautomer, stereoisomer, isotopically labeled derivative, or prodrug thereof, wherein R¹³, R¹⁵, and R¹⁷ are each H.

28. The heparin oligomer of claim 25, or a pharmaceutically acceptable salt, solvate, hydrate, polymorph, co-crystal, tautomer, stereoisomer, isotopically labeled derivative, or prodrug thereof, wherein each R¹⁴ is OH or wherein each R¹⁶ is OH.

29. (canceled)

30. The heparin oligomer of claim 25, or a pharmaceutically acceptable salt, solvate, hydrate, polymorph, co-crystal, tautomer, stereoisomer, isotopically labeled derivative, or prodrug thereof, wherein each R^{H} is —C(═O)CH₃.

31. The heparin oligomer of claim 25, or a pharmaceutically acceptable salt, solvate, hydrate, polymorph, co-crystal, tautomer, stereoisomer, isotopically labeled derivative, or prodrug thereof, wherein R^{G} is C(═O)-(substituted alkyl), wherein substituted alkyl is alkyl substituted with —N₃ or —C≡C—H.

32. The heparin oligomer of claim 25, or a pharmaceutically acceptable salt, solvate, hydrate, polymorph, co-crystal, tautomer, stereoisomer, isotopically labeled derivative or prodrug thereof, wherein R^{G} is optionally substituted alkyl or optionally substituted acyl, wherein the optionally substituted alkyl or optionally substituted acyl is alkyl or acyl substituted with a linker, wherein said linker is covalently bonded to an optionally substituted oligosaccharide.

33. The heparin oligomer of claim 32, or a pharmaceutically acceptable salt, solvate, hydrate, polymorph, co-crystal, tautomer, stereoisomer, isotopically labeled derivative or prodrug thereof, wherein the linker is —NHC(═O)—, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or any combination thereof.

34. The heparin oligomer of claim 32, or a pharmaceutically acceptable salt, solvate, hydrate, polymorph, co-crystal, tautomer, stereoisomer, isotopically labeled derivative, or prodrug thereof, wherein the linker is covalently bonded to an optionally substituted oligosaccharide having a structure of Formula (II): 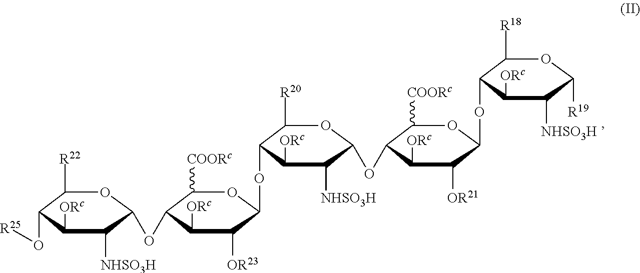
wherein:
each of R¹³, R²⁰, and R²² is independently —H, —OH, —OSO₃H, or —SO₃H;
each of R²¹ and R²³ is independently —SO₃H, —H, optionally substituted C₁-C₆ alkyl, or an oxygen protecting group;
each occurrence of R^{C} is independently —H, optionally substituted C₁-C₆ alkyl, or an oxygen protecting group;
R¹⁹ is a covalent bond to the linker or a bivalent group covalently bonded to the linker, wherein the bivalent group covalently bonded to the linker is selected from —O—R²⁶—, —SR²⁶—, —N(R^{B})(R²⁶)—, an optionally substituted monosaccharide residue covalently bonded to the linker, and an optionally substituted oligosaccharide residue covalently bonded to the linker;
R²⁶ is a covalent bond to the linker, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, or optionally substituted heteroarylene;
R^{B} is —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group when attached to a nitrogen atom; or
wherein R^{B} and R²⁶ together with the nitrogen atom to which they are attached form an optionally substituted heterocyclic ring or optionally substituted heteroaryl ring that comprises a covalent bond to the linker; and
R²⁵ is —H, optionally substituted alkyl, optionally substituted aralkyl, optionally substituted aryl, an optionally substituted monosaccharide, or an optionally substituted oligosaccharide.

35. The heparin oligomer of claim 32, wherein R²⁵ comprises one or more optionally substituted galactosamine residues.

36. The heparin oligomer of claim 1, or a pharmaceutically acceptable salt, solvate, hydrate, polymorph, co-crystal, tautomer, stereoisomer, isotopically labeled derivative, or prodrug thereof, wherein R⁵ is —OH or —O(oxygen protecting group).

37. The heparin oligomer of claim 1, or a pharmaceutically acceptable salt, solvate, hydrate, polymorph, co-crystal, tautomer, stereoisomer, isotopically labeled derivative, or prodrug thereof, wherein R⁵ is an optionally substituted monosaccharide.

38. The heparin oligomer of claim 37, or a pharmaceutically acceptable salt, solvate, hydrate, polymorph, co-crystal, tautomer, stereoisomer, isotopically labeled derivative, or prodrug thereof, wherein R⁵ is an optionally substituted glucuronide.

39. The heparin oligomer of claim 37, or a pharmaceutically acceptable salt, solvate, hydrate, polymorph, co-crystal, tautomer, stereoisomer, isotopically labeled derivative, or prodrug thereof, wherein R⁵ is 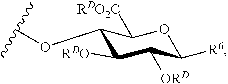
wherein:
R^{D} is —H, optionally substituted C₁-C₆ alkyl, or an oxygen protecting group;
R⁵ is —OR^{E}, —SR^{E}, or —N(R^{E})₂; and
each occurrence of R^{E} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{E} are joined together with their intervening atoms to form an optionally substituted heterocyclic ring or optionally substituted heteroaryl ring.

40. The heparin oligomer of claim 39, or a pharmaceutically acceptable salt, solvate, hydrate, polymorph, co-crystal, tautomer, stereoisomer, isotopically labeled derivative, or prodrug thereof, wherein R⁶ is —OR^{E} or —SR^{E}.

41. The heparin oligomer of claim 39, or a pharmaceutically acceptable salt, solvate, hydrate, polymorph, co-crystal, tautomer, stereoisomer, isotopically labeled derivative, or prodrug thereof, wherein R⁶ is —O— (optionally substituted phenyl).

42. The heparin oligomer of claim 18, or a pharmaceutically acceptable salt, solvate, hydrate, polymorph, co-crystal, tautomer, stereoisomer, isotopically labeled derivative, or prodrug thereof, wherein R⁶ is 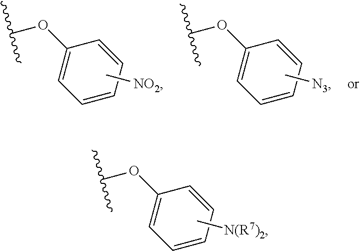
wherein:
each occurrence of R⁷ is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or two occurrences of R⁷ are joined together with their intervening atoms to form an optionally substituted heterocyclic ring or optionally substituted heteroaryl ring.

43. The heparin oligomer of claim 42, or a pharmaceutically acceptable salt, solvate, hydrate, polymorph, co-crystal, tautomer, stereoisomer, isotopically labeled derivative, or prodrug thereof, wherein R⁶ is

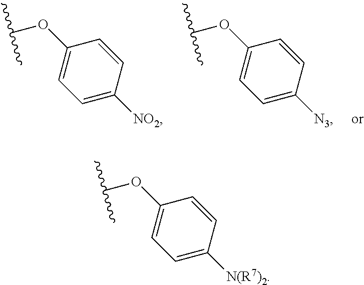

44. The heparin oligomer of claim 5, wherein the heparin oligomer is a heparin heptamer having the structure: 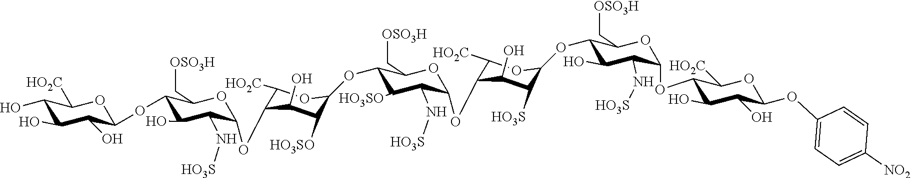
or a pharmaceutically acceptable salt, solvate, hydrate, polymorph, co-crystal, tautomer, stereoisomer, isotopically labeled derivative, or prodrug thereof.

45. The heparin oligomer of claim 1, wherein the heparin oligomer has the structure of compound 3 or compound 6, or a pharmaceutically acceptable salt, solvate, hydrate, polymorph, co-crystal, tautomer, stereoisomer, isotopically labeled derivative, or prodrug thereof, wherein the structure of compound 3 is 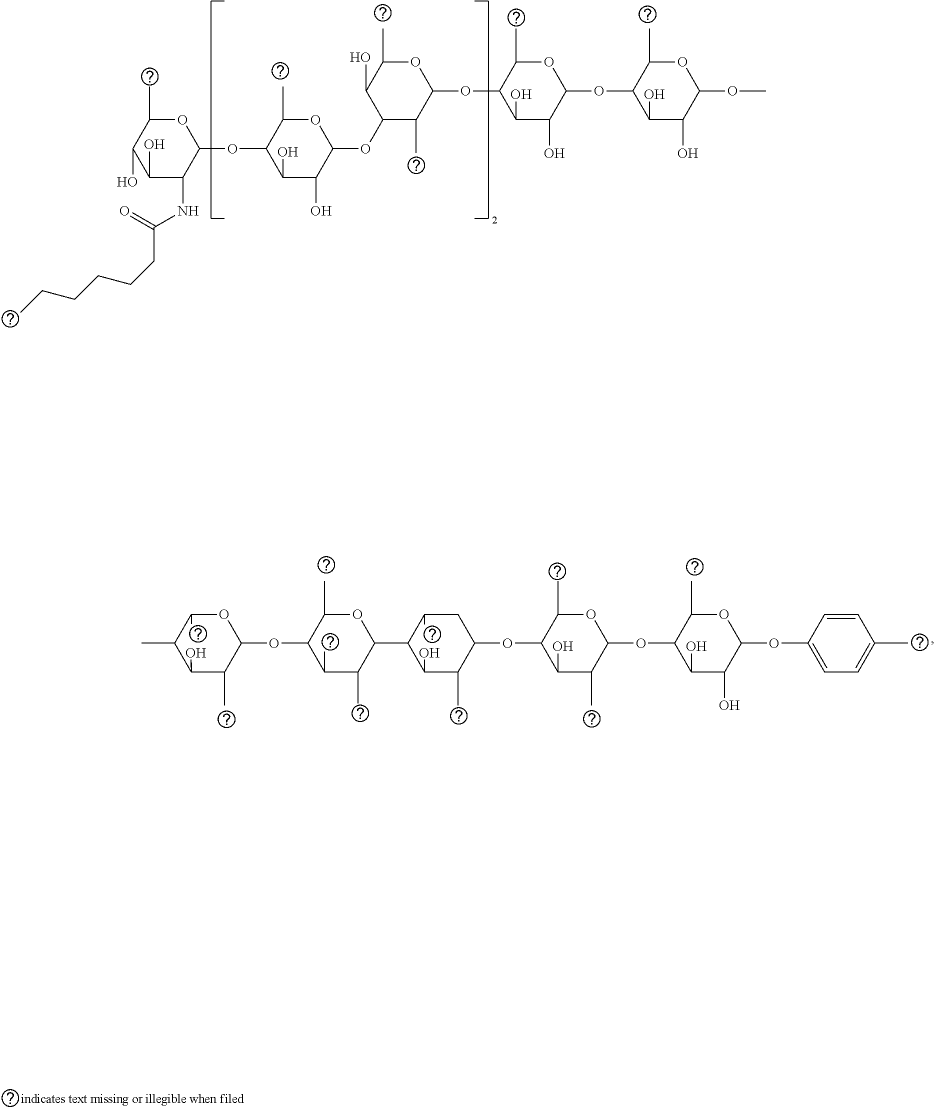
and the structure of compound 6 is:

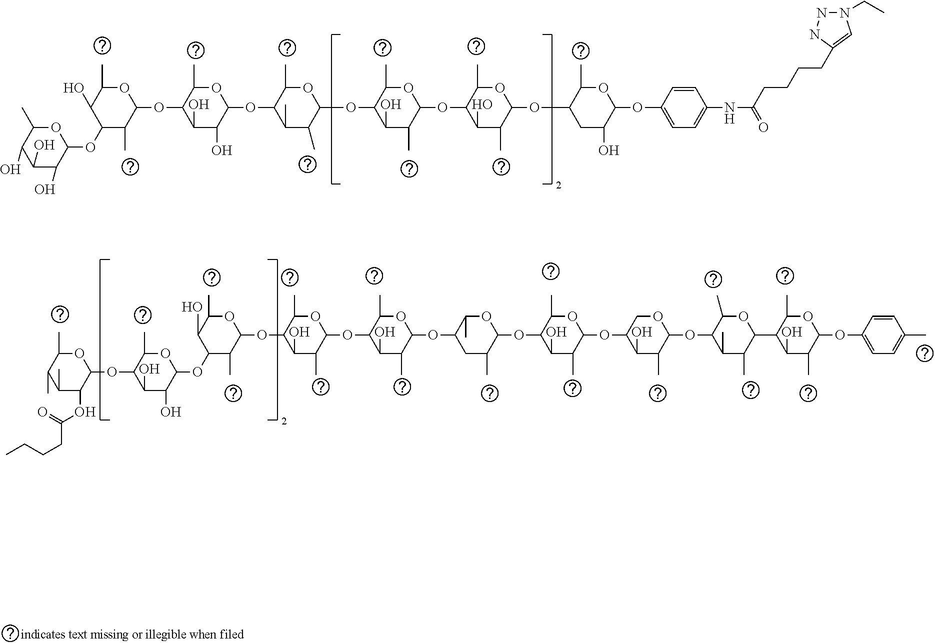

46. The heparin oligomer of claim 1, or a pharmaceutically acceptable salt, solvate, hydrate, polymorph, co-crystal, tautomer, stereoisomer, isotopically labeled derivative, or prodrug thereof, wherein the heparin oligomer is resistant to heparanase degradation.

47. The heparin oligomer of claim 1, or a pharmaceutically acceptable salt, solvate, hydrate, polymorph, co-crystal, tautomer, stereoisomer, isotopically labeled derivative, or prodrug thereof, wherein the heparin oligomer has anti-FXa activity and/or anti-FIIa activity.

48. An oligomeric compound comprising two or more repeat units connected via a linker, or a pharmaceutically acceptable salt, solvate, hydrate, polymorph, co-crystal, tautomer, stereoisomer, isotopically labeled derivative, or prodrug thereof, wherein the repeat units are each independently a heparin oligomer of claim 1.

49. The oligomeric compound of claim 48, or a pharmaceutically acceptable salt, solvate, hydrate, polymorph, co-crystal, tautomer, stereoisomer, isotopically labeled derivative, or prodrug thereof, wherein the linker is a bond, an optionally substituted saccharide, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or any combination thereof.

50. The oligomeric compound of claim 48, or a pharmaceutically acceptable salt, solvate, hydrate, polymorph, co-crystal, tautomer, stereoisomer, isotopically labeled derivative, or prodrug thereof, wherein the oligomeric compound has a linear structure.

51. The oligomeric compound of claim 48, or a pharmaceutically acceptable salt, solvate, hydrate, polymorph, co-crystal, tautomer, stereoisomer, isotopically labeled derivative, or prodrug thereof, wherein the oligomeric compound comprises five or more heparin oligomers.

52. A polymer conjugate, or a pharmaceutically acceptable salt, solvate, hydrate, polymorph, co-crystal, tautomer, stereoisomer, isotopically labeled derivative, or prodrug thereof, comprising a heparin oligomer of claim 1, or a pharmaceutically acceptable salt, solvate, hydrate, polymorph, co-crystal, tautomer, stereoisomer, isotopically labeled derivative, or prodrug thereof, conjugated to a polymer via a linker.

53. The polymer conjugate of claim 52, wherein the linker is a bond, an optionally substituted monosaccharide, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or any combination thereof.

54. The polymer conjugate of claim 52, wherein the linker is a bond, —NHC(═O)—, or an optionally substituted saccharide.

55. The polymer conjugate of claim 49, wherein the polymer is conjugated to either terminus of the heparin oligomer.

56. The polymer conjugate of claim 55, wherein R⁶ of the heparin oligomer is —O(optionally substituted phenyl) substituted with the linker.

57. The polymer conjugate of claim 55, having the structure 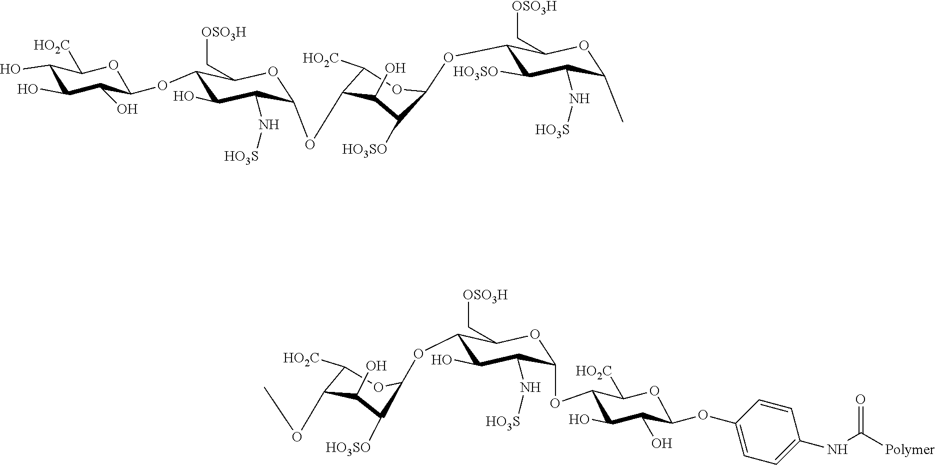
or a pharmaceutically acceptable salt, solvate, hydrate, polymorph, co-crystal, tautomer, stereoisomer, isotopically labeled derivative, or prodrug thereof.

58. The polymer conjugate of claim 52, having the structure 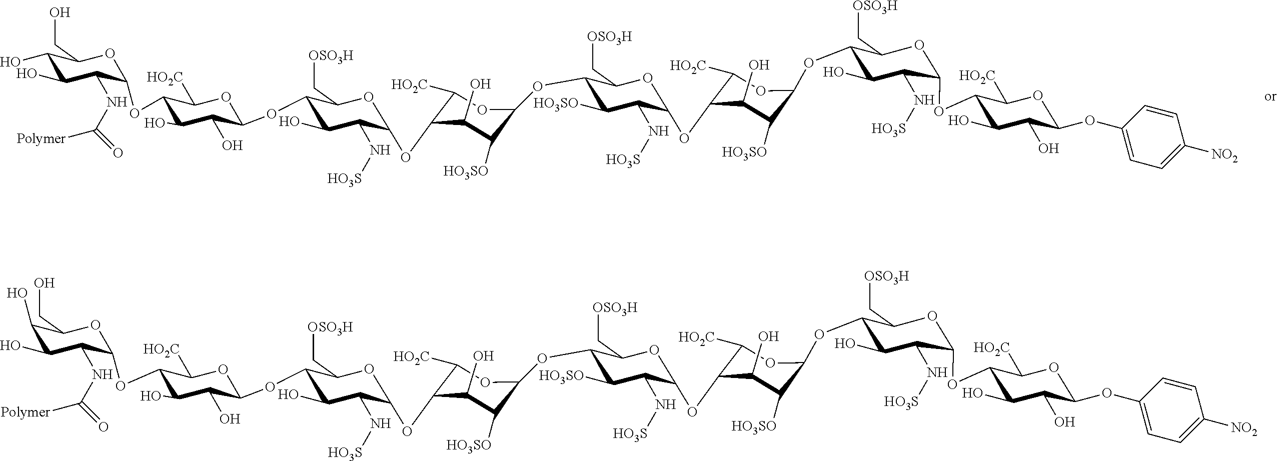
or a pharmaceutically acceptable salt, solvate, hydrate, polymorph, co-crystal, tautomer, stereoisomer, isotopically labeled derivative, or prodrug thereof.

59. The polymer conjugate of claim 52, wherein the polymer is a polyethylene glycol, a polyacrylate, a polyester, a polycarbonate, a polyolefin, a polyamide, or any combination thereof.

60. (canceled)

61. (canceled)

62. An oligosaccharide conjugate comprising the structure: 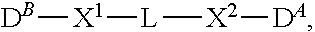
wherein:
L is a bivalent linker;
X¹ is present or absent and when present is an optionally substituted monosaccharide residue or an optionally substituted oligosaccharide residue;
X² is present or absent and when present is an optionally substituted monosaccharide residue or an optionally substituted oligosaccharide residue;
D^{A} is a heparin oligomer having a structure of Formula (I-B): 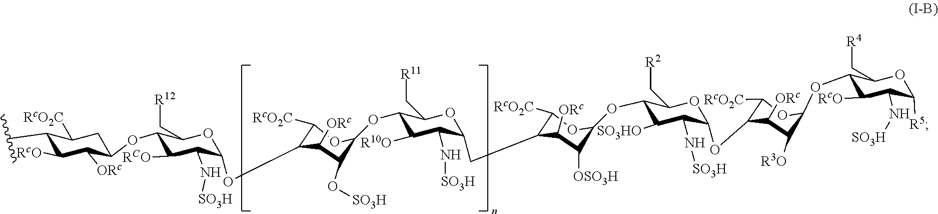
D^{B} is an oligosaccharide-containing oligomer having a structure of Formula (II-A): 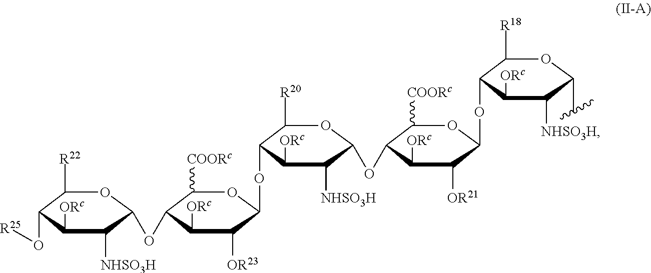
n is 1, 2, 3, 4, 5, 6, 7, 8, 8, 9, or 10;
each of R², R⁴, R¹¹, R¹², R¹⁸, R²⁰, and R²² is independently —H, —OH, —OSO₃H, or —SO₃H;
each of R³, R¹⁰, R²¹, and R²³ is independently —SO₃H, —H, optionally substituted C₁-C₆ alkyl, or an oxygen protecting group; and
R⁵ is —OR^{B}, —SR^{B}, —N(R^{B})₂, halogen, an optionally substituted monosaccharide, or an optionally substituted oligosaccharide;
each occurrence of R^{B} is independently —H, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{B} are joined together with their intervening atoms to form an optionally substituted heterocyclic ring or optionally substituted heteroaryl ring;
each occurrence of R^{C} is independently —H, optionally substituted C₁-C₆ alkyl, or an oxygen protecting group; and
R²⁵ is —H, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aralkyl, optionally substituted aryl, optionally substituted heteroaryl, an oxygen protecting group, an optionally substituted monosaccharide, or an optionally substituted oligosaccharide.

63. The oligosaccharide conjugate of claim 62, wherein L is an optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or any combination thereof.

64. The oligosaccharide conjugate of claim 62, wherein L is:
—O-phenyl-NH—C(═O)-(alkylene)-(triazolyl)-(alkylene)-C(═O)—.

65. The oligosaccharide conjugate of claim 62, wherein X² is present and comprises one or more optionally substituted galactosamine residues.

66. The oligosaccharide conjugate of claim 62, wherein R²⁵ is an optionally substituted oligosaccharide comprising one or more optionally substituted galactosamine residues.

67. The oligosaccharide conjugate of claim 62, wherein D^{A} comprises the structure:

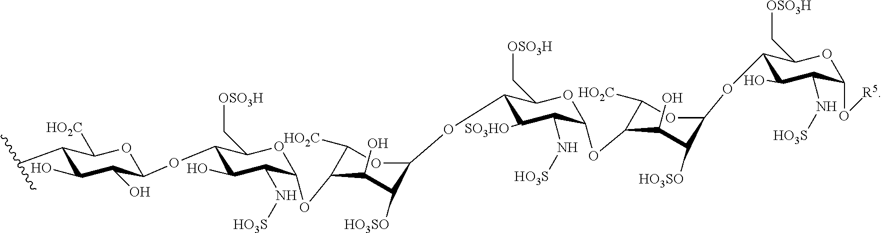

68. The oligosaccharide conjugate of claim 62, wherein D^{B} comprises the structure:

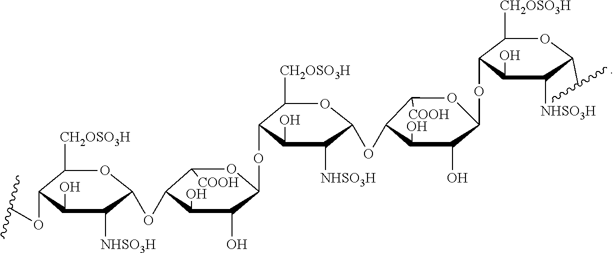

69. The oligosaccharide conjugate of claim 62, wherein the conjugate has the structure:

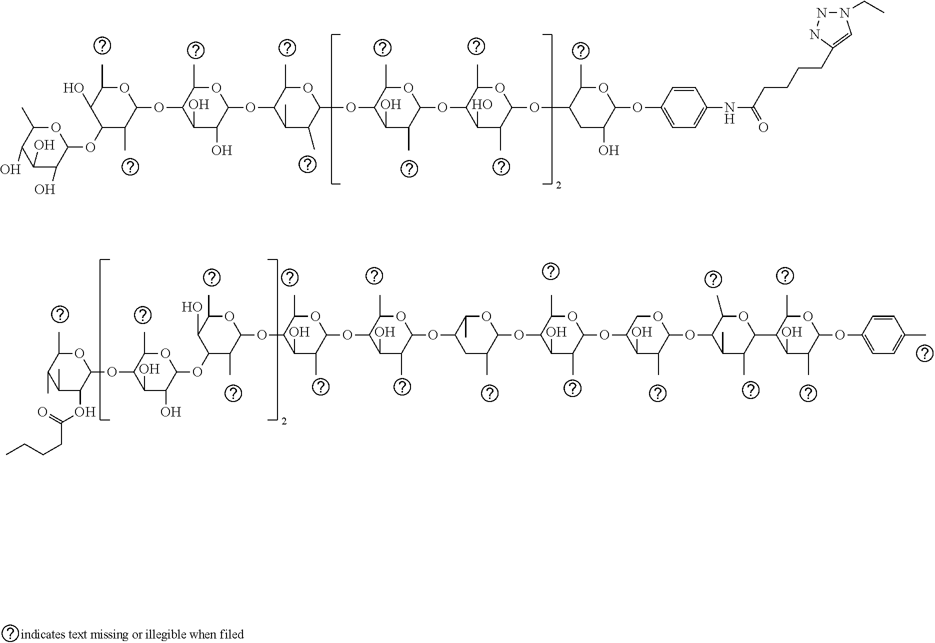

70. A method of synthesizing a heparin oligomer of claim 1, or a pharmaceutically acceptable salt, solvate, hydrate, polymorph, co-crystal, tautomer, stereoisomer, isotopically labeled derivative, or prodrug thereof, comprising the sequential steps of:
(a) elongating a saccharide using:
(i) recombinant Pasteurella multocida heparosan synthase (pmHS2) and uridine 5-diphospho-N-trifluoroacetyl glucosamine (UDP-GlcNTFA); and
(ii) recombinant Pasteurella multocida heparosan synthase (pmHS2) and uridine 5-diphospho-N-glucuronic acid (UDP-GlcA)
in either order, one or more times, to obtain a trifluoroacetate-protected heparin hexamer oligosaccharide intermediate comprising the structure 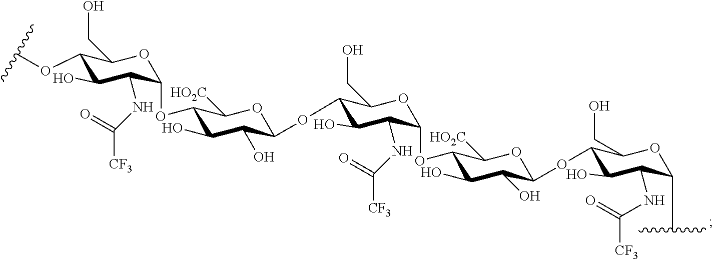
(b) detrifluoroacetylating the heparin hexamer oligosaccharide intermediate of step (a) to obtain a heparin hexamer oligosaccharide intermediate comprising the structure 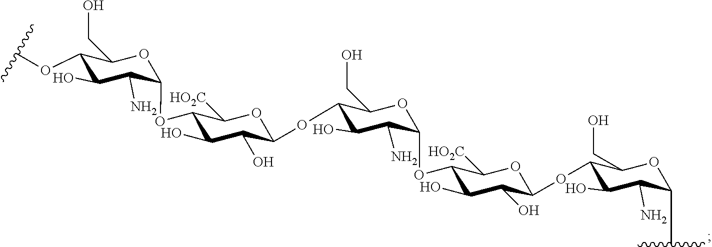
(c) N-sulfating the heparin hexamer oligosaccharide intermediate of step (b) to obtain an NS-hexamer oligosaccharide intermediate comprising the structure 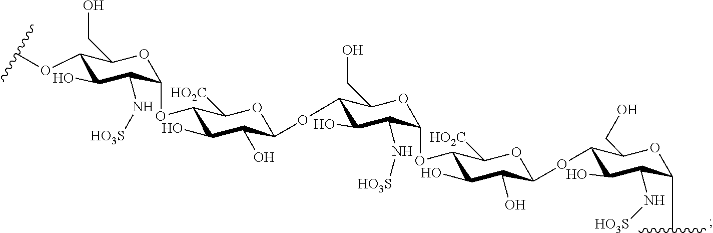
(d) elongating the NS-hexamer oligosaccharide intermediate of step (c) with recombinant Pasteurella multocida heparosan synthase (pmHS2) and uridine 5-diphospho-N-glucuronic acid (UDP-GlcA) to obtain an oligosaccharide intermediate comprising the structure 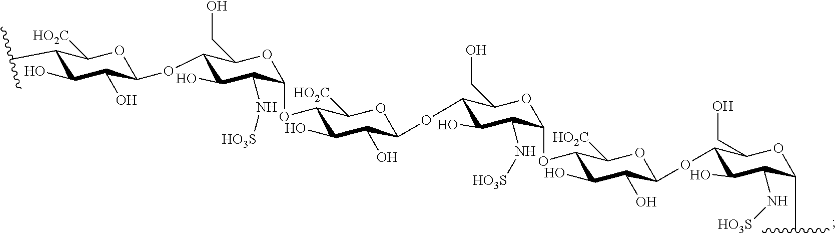
(e) converting glucuronic acid to 2-O-sulfated iduronic acid to obtain an oligosaccharide intermediate comprising the structure 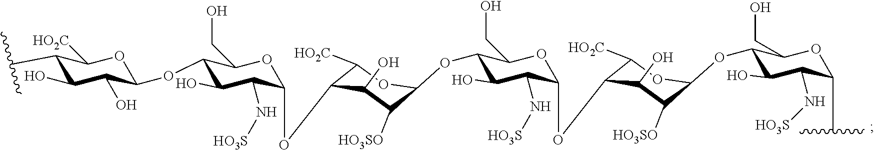
and optionally
(f) performing 3-O- and/or 6-O sulfation of the oligosaccharide intermediate of step (e).

71. The method of claim 70, wherein the saccharide of step (a) is para-nitrophenyl glucuronide.

72. The method of claim 70, wherein step (b) comprises reaction under basic conditions.

73. The method of claim 70, wherein step (c) comprises incubation with 3-morpholino-propane-1-sulfonic acid, N-sulfotransferase, and 3′-phosphoadenosine 5′-phosphosulfate.

74. The method of claim 70, wherein step (e) comprises incubation with C₅-epimerase, 2-O-sulfotransferase, and 3′-phosphoadenosine 5′-phosphosulfate in 3-morpholino-propane-1-sulfonic acid buffer.

75. The method of claim 70, wherein step (f) comprises incubation with 3-O-sulfotransferase 3 and 3′-phosphoadenosine 5′-phosphosulfate and/or incubation with 6-sulfotransferase 3 in 3-morpholino-propane-1-sulfonic acid buffer.

76. The method of claim 70, wherein any of steps (a)-(f) is followed by an additional purification step.

77. The method of claim 70, wherein the heparin oligomer is synthesized in a final yield of about 45% over all steps.

78. A pharmaceutical composition comprising a heparin oligomer of claim 1, or a pharmaceutically acceptable salt, solvate, hydrate, polymorph, co-crystal, tautomer, stereoisomer, isotopically labeled derivative, or prodrug thereof, and a pharmaceutically acceptable excipient.

79. The pharmaceutical composition of claim 78, wherein the pharmaceutical composition is formulated for oral administration, wherein the pharmaceutical composition is formulated for intravenous or subcutaneous administration or wherein the pharmaceutical composition further comprises an additional therapeutic agent.

80. (canceled)

81. (canceled)

82. A surface coating comprising a heparin oligomer of claim 1, or a pharmaceutically acceptable salt, solvate, hydrate, polymorph, co-crystal, tautomer, stereoisomer, isotopically labeled derivative, or prodrug thereof, and an excipient.

83. A device comprising a surface coating of claim 82.

84. The device of claim 83, wherein the device is an implantable medical device, or wherein the device is a vascular graft, a stent, a cardiopulmonary bypass circuit, a ventricular assist device, or a respiratory support system.

85. (canceled)

86. A method of treating or preventing a disease in a subject in need thereof, comprising administering to the subject an effective amount of a heparin oligomer of claim 1, or a pharmaceutically acceptable salt, solvate, hydrate, polymorph, co-crystal, tautomer, stereoisomer, isotopically labeled derivative, or prodrug thereof, or a pharmaceutical composition thereof.

87. The method of claim 86, wherein the disease is cardiovascular disease, atherosclerosis, atherosclerotic lesions, thrombus formation, thromboembolism, stroke, or myocardial infarction.

88. The method of claim 86, wherein the disease is primary or recurrent venous thromboembolism (VTE).

89. The method of claim 88, wherein the venous thromboembolism is deep vein thrombosis, pulmonary embolism, or non-occlusive venous thrombosis.

90.-96. (canceled)

97. The method of claim 86, further comprising administering an additional therapy or therapeutic agent to the subject before, concurrently with, or after administering the effective amount of the heparin oligomer; pharmaceutically acceptable salt, solvate, hydrate, polymorph, co-crystal, tautomer, stereoisomer, isotopically labeled heparin oligosaccharide, or prodrug thereof; or pharmaceutical composition thereof.

98.-102. (canceled)
